(19) United States
(12) Patent Application Publication
(10) Pub. No.: US 2026/0049314 A1
(43) Pub. Date: Feb. 19, 2026

(54) ANTISENSE OLIGONUCLEOTIDE-BASED ANTI-FIBROTIC THERAPEUTICS

(71) Applicant: UNIVERSITY OF ROCHESTER, ROCHESTER, NY (US)

(72) Inventors: Peng YAO, Pittsford, NY (US); Omar HEDAYA, Rochester, NY (US)

(73) Assignee: UNIVERSITY OF ROCHESTER, ROCHESTER, NY (US)

(21) Appl. No.: 19/102,137

(22) PCT Filed: Aug. 23, 2023
(86) PCT No.: PCT/US2023/072713

§ 371 Date: Feb. 7, 2025

Related U.S. Application Data
(60) Provisional application No. 63/373,796, filed on Aug. 29, 2022.

(51) Int. Cl. C12N 15/113; A61K 31/711; A61K 31/712; A61P 9/00

(52) U.S. Cl. CPC C12N 15/1136; A61K 31/711; A61K 31/712; A61P 9/00; C12N 2310/11; C12N 2310/14; C12N 2310/315; C12N 2310/321; C12N 2310/3231; C12N 2310/341

(57) ABSTRACT

The present application provides antisense oligonucleotides capable of enhancing expression of an anti-fibrosis gene or reducing expression of a pro-fibrosis gene in a target tissue. The antisense oligonucleotides may be used for the treatment of cardiac fibrosis. The antisense oligonucleotide may include a gapmer capable of binding to, and forming a doublestranded structure with, a target sequence in a mRNA of a pro-fibrosis gene.

This application claims priority from U.S. Provisional Patent Application No. 63/373,796, filed on Aug. 29, 2022, which is incorporated herein by reference.

This invention was made with government support under HL132899 and HL147954 awarded by the National Institutes of Health. The government has certain rights in the invention.

### FIELD

This application relates to the field of medical treatment. More specifically, the present invention provides compositions and methods useful for treating fibrosis-related diseases and conditions.

### BACKGROUND

Fibrosis is a physiological process in which connective tissue replaces normal parenchymal tissue. Fibrosis can occur in many tissues within the body, such as heart, lung, liver and kidney, typically as a result of inflammation, tissue injury or aging. However, the entire process can lead to a progressive irreversible fibrotic response if tissue injury is severe or repetitive, or if the fibrosis process itself becomes deregulated. Fibrotic diseases cause annually more than 800,000 deaths worldwide, mostly due to lung and cardiac fibrosis. We will further summarize current diagnostic tools and highlight pre-clinical or clinical therapeutic strategies to address cardiac fibrosis.

Therefore, there exists a need to develop therapeutic strategies to address fibrosis-related diseases, such as cardiac fibrosis.

One of the keyways to increase the production of a therapeutic protein is through a gene therapy approach. AAV delivery of transgenes has had challenges with toxicity, and lipid nanoparticle (LNP) delivery is currently being refined within the industry. Therefore, there is a need to look for platforms that could potently, safely, and in a titratable manner increase protein synthesis.

### SUMMARY

One aspect of the application relates to an antisense oligonucleotide comprising 8-50 nucleotides, wherein the antisense oligonucleotide is capable of binding to, and forming a double-stranded structure with, a target sequence in a mRNA of an anti-fibrosis gene, wherein the target sequence is located in a non-coding strand of a double-stranded stem structure downstream of, and adjacent to, a uORF start codon in the mRNA, and wherein the binding of the antisense oligonucleotide to the target sequence disrupts the double-stranded stem structure of the uORF and enhances translation of a mORF of the mRNA.

Another aspect of the application relates to an antisense oligonucleotide comprising 8-50 nucleotides, wherein the antisense oligonucleotide is capable of binding to, and forming a double-stranded structure with, a target sequence in a mRNA of an anti-fibrosis gene, wherein the target sequence is located downstream of, and adjacent to, a mORF start codon in the mRNA, and wherein the binding of the antisense oligonucleotide to the target sequence enhances translation from the mORF start codon.

Another aspect of the application relates to an antisense oligonucleotide comprising 8-50 nucleotides, wherein the antisense oligonucleotide is capable of binding to, and forming a double-stranded structure with, a target sequence in a mRNA of a pro-fibrosis gene, wherein the target sequence is located downstream of, and adjacent to, an uORF start codon in the mRNA, and wherein the binding of the antisense oligonucleotide to the target sequence reduces translation from the mORF start codon.

Another aspect of the application relates to an antisense oligonucleotide comprising 8-50 nucleotides, wherein the antisense oligonucleotide is a gapmer capable of binding to, and forming a double-stranded structure with, a target sequence in a mRNA of a pro-fibrosis gene, wherein the target sequence is located in a region that spans from 55 nucleotides upstream of an uORF start codon to 55 nucleotides downstream of the uORF start codon in the mRNA, and wherein the binding of the antisense oligonucleotide to the target sequence reduces translation from the mORF start codon and also degrade the target mRNA.

Another aspect of the application is a pharmaceutical composition for anti-fibrosis therapy, comprising: the antisense oligonucleotide as described herein; and a pharmaceutically acceptable carrier.

Another aspect of the application is a method for treating cardiac fibrosis, comprising administering in a subject in need thereof, an effective amount of the antisense oligonucleotide described herein or an effective amount of the pharmaceutical composition as described herein.

### BRIEF DESCRIPTION OF THE DRAWINGS

FIG. 1 shows models for translational activation or suppression. (A) shows a model for Type I uotASO suppression of uORF translation and activation of mORF translation. (B) shows a model for Type II uotASO-targeted activation of uORF translation and suppression of mORF translation. This design does not rely on the presence of an endogenous dsRNA stem structure and would activate translation of those uORFs that do not contain an endogenous dsRNA stem structure. (C) shows a model for Type II motASO-targeted activation of mORF translation.

FIG. 2 is a composite of drawings and pictures showing crosstalk between uORF and an adjacent double-stranded RNA structural element in translational regulation of mORF translation. Panel A) Schematic of the dual luciferase reporter assay. Panel B) Schematic of FLuc reporter constructs. Panel C) Dual luciferase reporter assay using a series of constructs that contain uORF start codon and adjacent dsRNA structure (Kan-HP1 hairpin) that is located at different distances. N=3 biological replicates. Data were presented as mean±SEM. P values were calculated by unpaired two-tailed Student t test. Panel D) Dual luciferase reporter assay using mutant construct that contains three nucleotide mutations leading to disrupted stem structure. N=3 biological replicates. Data were presented as mean SEM. P values were calculated by unpaired two-tailed Student t test. No AUG: ATG-to-TTG mutation. AUG −2: start codon is located at −2 position relative to the hairpin. Intact: stable WT hairpin. Weakened: three mutations introduced in the hairpin to destabilize the structure.

FIG. 3 shows localization of artificial uORF-KanHP1 mRNA variants in 40S ribosomal subunit or 80S monosome fractions in HEK293T lysates upon 10-35% sucrose gradient centrifugation. Experiments were repeated 2 times, and representative data were shown.

FIG. 4 is a composite of drawings and pictures identifying uORFs as major translational regulatory elements in mRNAs encoding cardiac transcription factors. Panel A) illustrates the overlap of mRNAs containing uORFs based on ribosome profiling (Ribo-Seq) in human and mouse failing hearts along with an ontological analysis of human cardiac uORFs. Multiple cardiac mRNAs and embedded uORFs are highlighted such as GATA4. Panel B) GATA4 uORFs are present across mammals as shown in a representative group of species. Panel C) Schematic of WT and mutant GATA4 5′ UTR cloned in FLuc reporter constructs. Panel D) dsRNA element is required for uORF-mediated translational repression of mORF. Dual luciferase reporter assay with WT, ΔuORF, secondary structure mutant, and rescuing mutant. N=3 biological replicates. Data were presented as mean±SEM. P values were calculated by unpaired two-tailed Student t test.

FIG. 5 is a composite of drawings and pictures showing a mechanism-based design of ASOs for regulating mORF translation. Panels A-B, left show a schematic of designed ASOs targeting the GATA4 uORF dsRNA element. ASOs that are disruptive to the formation of double-stranded RNA structure immediately downstream of the AUG start codon of the GATA4 uORF are designated as type I uotASOs or ASO1 (Panel A). ASOs that are capable of forming a double-stranded RNA structure immediately downstream of the AUG start codon of the GATA4 uORF are designated as type II uotASOs or ASO2 (Panel B). Panels A-B, middle show dual luciferase reporter assays with WT and ΔuORF mutant GATA4 after transfection of ASO1 and ASO2 (oligo sequences are shown in Panel F). N=3 biological replicates. Comparisons were performed by unpaired two-tailed Student t test. Panel C) Western blot analysis of dose-responsive manipulation of endogenous GATA4 protein expression by ASO1 and ASO2 in AC16 human cardiomyocyte cell line. Panels D-E) Polysome profiling of WT and ΔuORF cells with ASO1/ASO2 treatment in AC16 cells. Panel F) b-actin immunostaining of AC16 cells after transfection of control ASO, ASO1 and ASO2. Cell surface area was measured and quantified (n≥200 cells). Scale bar: 20 mm. In the violin plot, solid line shows median value for the group and dashed lines represent two quartile lines in each group. P values were calculated by unpaired two-tailed Student t test.

FIG. 6 is a development of type II ASOs to inhibit pro-fibrotic eIF4G2 expression (with type II uotASO) or enhance anti-fibrotic GATA4, MEF2C and NKX2-5 expression (with type II motASOs). Panel A: Western blot analysis of eIF4G2 protein upon transfection of 50 nM uORF-enhancing ASOs with modifications for eIF4G2 mRNA. Panel B: GATA4 mORF targeting ASOs enhance its protein levels. Like with uORF-targeting ASOs, the combination of 2′-O-methyl and LNA is superior to 2′-O-methyl alone and does not change mRNA levels (Panel C). Panel D: 2′-O-methyl and LNA mORF-targeting ASOs (50 nM) increase the protein levels of mRNA with cognate start codons in the case of MEF2C and NKX2-5. Data are represented as mean SD. * P<0.05, ** P<0.01, ** P<0.001. Statistical significance was confirmed by an unpaired two-tailed Student t test for A-D (N=3 biological replicates)

FIG. 7 shows the description of the design strategy of ASOs targeting different mRNAs. A. Schematic of mORF-activating Type II motASOs and 5′ UTR-targeting gapmer ASOs. B. Detailed information on therapeutic target mRNAs, specific ASO sequences, and chemical modifications.

FIG. 8 shows the translation activation effects of Type II motASOs targeting MYBPC3 and CRYAB. Panel A. Dose-dependent effects of ASO targeting MYBPC3 mRNA in human AC16 ventricular cardiomyocyte cell line. Western blot was performed 24 hours after the transfection of the ASO using 0.4% lipofectamine 3000 at incremental doses. Panel B. Dose-dependent effects of ASO targeting CRYAB mRNA in human AC16 ventricular cardiomyocyte cell line. Panel C. MYBPC3 and CRYAB mRNA expression in AC16 cells under transfection of incremental dose of ASO. The experiments were repeated twice (biological replicates), and representative western blot data was shown. Densitometry analysis data was shown between the blot images and calculated as a ratio of target protein normalized by α-tubulin as an internal loading control.

FIG. 9 shows mRNA degradation and protein expression silencing effects of 5′ UTR-targeting Gapmer ASO targeting EIF4G2 mRNA. Panel A. Downregulation effects of ASO targeting EIF4G2 mRNA in human immortalized cardiac fibroblast (IHCF) cell line. Western blot was performed 48 hours after the transfection of 100 nmol ASO using 0.3% RNAiMAX. The observed molecular weight of EIF4G2 is ˜97 kDa (theoretical MW is 102 kDa). The experiments were repeated three times (biological triplicates), and representative western blot data was shown (technical triplicates included). Panel B. Quantitative densitometry analysis data was calculated as a ratio of target protein normalized by α-tubulin as an internal loading control. Panel C. Reduced EIF4G2 mRNA expression in IHCF cells with ASO transfection.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” are also disclosed, as appropriately understood by the skilled artisan. For example, if the value “10” is disclosed, the “less than or equal to 10” and “greater or equal to 10” is also disclosed. When two or more value are disclosed, all possible ranges between any two values are disclosed.

### I. Definitions

As used herein, the following terms or phrases (in parentheses) shall have the following meanings:

A “target protein” refers to a protein that one desires to increase or decrease in amount, concentration, or activity. In certain embodiments, the target protein is encoded by the primary open reading frame of a target transcript.

A “main open reading frame” or “mORF” refers to the portion of the target transcript that encodes the main (or primary) protein associated with an mRNA transcript. In certain embodiments, the mORF encodes the target protein.

The terms “uORF” and “upstream open reading frame” refer to a portion of a target transcript that comprises a start site upstream of (i.e. 5′ of) the mORF and an in frame termination codon. In certain embodiments, a uORF is the portion of the target transcript that is translated when translation is initiated at a uORF start site. In certain embodiments, a uORF does not overlap with an mORF. In certain embodiments, a uORF overlaps with the mORF. In certain embodiments a uORF overlaps with another uORF. In certain embodiments, a uORF is out of frame with an mORF.

The term “oligonucleotide” refers to a compound comprising a plurality of linked nucleosides. In certain embodiments, an oligonucleotide comprises one or more unmodified ribonucleosides and/or unmodified deoxyribonucleosides and/or one or more modified nucleosides.

The term “oligonucleoside” refers to an oligonucleotide in which none of the internucleoside linkages contains a phosphorus atom. The term oligonucleotides include oligonucleosides.

The term “internucleoside linkage” refers to a covalent linkage between adjacent nucleosides in an oligonucleotide.

A “naturally occurring internucleoside linkage” refers to a 3′ to 5′ phosphodiester linkage.

The term “modified internucleoside linkage” refers to any internucleoside linkage other than a naturally occurring internucleoside linkage.

The terms “antisense oligonucleotide” or “ASO” refer to a compound comprising or consisting of an oligonucleotide or modified oligonucleotide at least a portion of which is complementary to a target nucleic acid, a target nucleotide sequence (target sequence), a target site of a nucleotide sequence (target site), or a target region of a nucleotide sequence (target region), to which it is capable of hybridizing, resulting in at least one antisense activity. In some embodiments, the ASO comprises a nucleotide sequence that is at least 50%, 60%, 70%, 80%, 90% or 95% complementary to the target sequence, the target site or the target region. In some embodiments, an ASO comprises an antisense oligonucleotide conjugated to a conjugate group. In some embodiments, the conjugate group is a non-nucleotide conjugate group.

An “antisense activity” refers to any detectable and/or measurable change attributable to the hybridization of an antisense oligonucleotide to its target nucleic acid, target sequence, target site or target region.

The term “GATA4 transcript” refers to a native GATA4 mRNA transcript encoding an upstream open reading frame (uORF) and a main open reading frame (mORF) encoding the GATA4 protein.

The term “nucleoside” refers to a molecule comprising a nucleobase moiety such as a purine or pyrimidine base covalently linked to a sugar moiety such as ribose or deoxyribose sugar. Nucleosides include, but are not limited to, naturally occurring nucleosides (as found in DNA and RNA) and modified nucleosides. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as “rare” nucleosides). Nucleosides may be linked to a phosphate moiety.

The term “modified nucleoside” refers to a nucleoside comprising at least one chemical modification compared to naturally occurring RNA or DNA nucleosides. Modified nucleosides comprise a modified sugar moiety and/or a modified nucleobase.

The term “nucleotide” refers to a nucleoside having one or more phosphate groups joined in ester linkages to the sugar moiety. Exemplary nucleotides include nucleoside monophosphates, diphosphates and triphosphates.” The term “linked nucleosides” are nucleosides that are connected in a continuous sequence (i.e., no additional nucleosides are present between those that are linked).

The term “modified nucleotide” refers to a nucleotide comprising a modified nucleoside with optional modifications in the phosphate linking group. In certain exemplary embodiments, the modified nucleotide is modified at any position so as to alter certain chemical properties of the nucleotide yet retain the ability of the modified nucleotide to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino) propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotides such as those described in Herdewijn, Antisense Nucleic Acid Drug Dev., 2000 Aug. 10(4):297-310. The modified nucleotide may also comprise modifications to the sugar moiety of the nucleotides. For example, the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438. The phosphate linking group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said modified nucleotides in vivo or in vitro.

The term “chemical modification” refers to a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

The term “furanosyl” refers to a structure comprising a 5-membered ring comprising four carbon atoms and one oxygen atom.

The phrase “naturally occurring sugar moiety” refers to a ribofuranosyl as found in naturally occurring RNA or a deoxyribofuranosyl as found in naturally occurring DNA.

The term “sugar moiety” refers to a naturally occurring sugar moiety or a modified sugar moiety of a nucleoside.

The term “modified sugar moiety” refers to a substituted sugar moiety or a sugar surrogate.

The term “substituted sugar moiety” refers to a furanosyl that is not a naturally occurring sugar moiety. Substituted sugar moieties include, but are not limited to furanosyls comprising substituents at the 2′-position, the 3′-position, the 5′-position and/or the 4′-position. Certain substituted sugar moieties are bicyclic sugar moieties.

The term “2′-substituted sugar moiety” refers to a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

The term “2′-F nucleoside” refers to a nucleoside comprising a sugar comprising fluoroine at the 2′ position. Unless otherwise indicated, the fluorine in a 2′-F nucleoside is in the ribo position (replacing the OH of a natural ribose).

The term “2′-(ara)-F” refers to a 2′-F substituted nucleoside, wherein the fluoro group is in the arabino position.

The term “sugar surrogate” refers to a structure that does not comprise a furanosyl and is capable of replacing the naturally occurring sugar moiety of a nucleoside, such that the resulting nucleoside sub-units are capable of linking together and/or linking to other nucleosides to form an oligonucleotide which is capable of hybridizing to a complementary oligonucleotide. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

The term “bicyclic sugar moiety” refers to a modified sugar moiety comprising a 4 to 7 membered ring (including but not limited to a furanosyl) comprising a bridge connecting two atoms of the 4 to 7 membered-ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

The term “nucleobase” refers to a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

The terms, “unmodified nucleobase” and “naturally occurring nucleobase” refer to the naturally occurring heterocyclic nucleobases of RNA or DNA: the purine bases adenine (A) and guanine (G), and the pyrimidine bases thymine (T), cytosine (C) (including 5-methyl C), and uracil (U).

The term “modified nucleobase” refers to any nucleobase that is not a naturally occurring nucleobase.

The term “bicyclic nucleoside” or “BNA” refers to a nucleoside comprising a bicyclic sugar moiety.

The terms “constrained ethyl nucleoside” and “cEt” refer to a nucleoside comprising a bicyclic sugar moiety comprising a 4′-CH(CH₃)—O-2′bridge.

The term “locked nucleic acid nucleoside” or “LNA” refers to a nucleoside comprising a bicyclic sugar moiety comprising a 4′-CH₂—O-2′bridge.

The term “2′-substituted nucleoside” refers to a nucleoside comprising a substituent at the 2′-position other than H or OH Unless otherwise indicated, a 2′-substituted nucleoside is not a bicyclic nucleoside.

The term “2′-deoxynucleoside” refers to a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

The term “oligomeric compound” refers to a polymeric structure comprising two or more sub-structures. In certain embodiments, the sub-structures are nucleotides or nucleosides. In certain embodiments, an oligomeric compound comprises an oligonucleotide. In certain embodiments, an oligomeric compound consists of an oligonucleotide. In certain embodiments, an oligomeric compound consists of an antisense oligonucleotide.

The term “terminal group” refers to one or more atom attached to either, or both, the 3′ end or the 5′ end of an oligonucleotide. In certain embodiments a terminal group is a conjugate group. In certain embodiments, a terminal group comprises one or more terminal group nucleosides.

The term “conjugate group” refers to an atom or group of atoms bound to an oligonucleotide or oligomeric compound. In general, conjugate groups modify one or more properties of the oligonucleotide or oligomeric compound to which they are attached, including, but not limited to pharmacodynamic, pharmacokinetic, binding, absorption, cellular distribution, cellular uptake, charge and/or clearance properties.

The term “conjugate linking group” refers to any atom or group of atoms used to attach a conjugate to an oligonucleotide or oligomeric compound.

The terms “detecting” and “measuring” refer to that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

The phrase “detectable and/or measurable activity” refers to a measurable activity that is not zero.

The term “essentially unchanged” refers to little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

The term “expression” refers to the process by which a gene ultimately results in a protein. Expression includes, but is not limited to, transcription, post-transcriptional modification (e.g., splicing, polyadenylation, addition of 5′-cap), translation, and post-translational modification.

The term “translation” refers to the process in which a polypeptide (e.g. a protein) is translated from an mRNA. In certain embodiments, an increase in translation refers to an increase in the number of polypeptide (e.g. a protein) molecules that are made per copy of mRNA that encodes said polypeptide.

The term “target nucleic acid” refers to a nucleic acid molecule to which an antisense oligonucleotide is intended to hybridize.

The term “mRNA” refers to an RNA molecule that encodes a protein.

The term “pre-mRNA” refers to an RNA transcript that has not been fully processed into mRNA. A pre-RNA may include one or more introns.

The terms “targeting” and “targeted to” refer to the association of an antisense oligonucleotide to a particular target nucleic acid molecule or a particular region of a target nucleic acid molecule. An antisense oligonucleotide targets a target nucleic acid if it is sufficiently complementary to the target nucleic acid to allow hybridization under physiological conditions.

When in reference to nucleobases, the terms “nucleobase complementarity” and “complementarity” refer to a nucleobase that is capable of base pairing with another nucleobase. For example, in DNA, adenine (A) is complementary to thymine (T). For example, in RNA, adenine (A) is complementary to uracil (U). Complementarity can be partial or total. Partial complementarity occurs when one or more nucleic acid bases is not matched according to the base pairing rules. Total or complete complementarity between nucleic acids occurs when each and every nucleic acid base is matched with another base under the base pairing rules. In certain embodiments, a complementary nucleobase refers to a nucleobase of an antisense oligonucleotide that is capable of base pairing with a nucleobase of its target nucleic acid. For example, if a nucleobase at a certain position of an antisense oligonucleotide is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

When used in reference to nucleobases, the term “non-complementary” refers to a pair of nucleobases that do not form hydrogen bonds with one another.

When used in reference to oligomeric compounds (e.g., linked nucleosides, oligonucleotides, or nucleic acids), the term “complementary” refers to the capacity of such oligomeric compounds or regions thereof to hybridize to another oligomeric compound or region thereof through nucleobase complementarity under stringent conditions. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary.

The term “mismatch” refers to a nucleotide of a first polynucleotide that is not capable of pairing with a nucleotide at a corresponding position of a second polynucleotide, when the first and second polynucleotide are aligned.

The term “hybridization” refers to the pairing of complementary oligomeric compounds (e.g., an antisense oligonucleotide and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

The term “specifically hybridizes” refers to the ability of an oligomeric compound to hybridize to one nucleic acid site with greater affinity than it hybridizes to another nucleic acid site. In certain embodiments, an antisense oligonucleotide specifically hybridizes to more than one target site.

When used in reference to an oligonucleotide or portion thereof, the term “fully complementary” means that each nucleobase of the oligonucleotide or portion thereof is capable of pairing with a nucleobase of a complementary nucleic acid or contiguous portion thereof. Thus, a fully complementary region comprises no mismatches or unhybridized nucleobases in either strand.

The term “percent complementarity” refers to the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

The term “percent identity” refers to the number of nucleobases in a first nucleic acid that are the same type (independent of chemical modification) as nucleobases at corresponding positions in a second nucleic acid, divided by the total number of nucleobases in the first nucleic acid.

The term “modulation” refers to a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

The term “modification motif” refers to a pattern of chemical modifications in an oligomeric compound or a region thereof. Motifs may be defined by modifications at certain nucleosides and/or at certain linking groups of an oligomeric compound.

The term “nucleoside motif” refers to a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

The term “sugar motif” refers to a pattern of sugar modifications in an oligomeric compound or a region thereof.

The term “linkage motif” refers to a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

The term “nucleobase modification motif” refers to a pattern of modifications to nucleobases along an oligonucleotide. Unless otherwise indicated, a nucleobase modification motif is independent of the nucleobase sequence.

The term “sequence motif” refers to a pattern of nucleobases arranged along an oligonucleotide or portion thereof. Unless otherwise indicated, a sequence motif is independent of chemical modifications and thus may have any combination of chemical modifications, including no chemical modifications.

The term “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

The term “differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

The term “the same type of modifications” refers to modifications that are the same as one another, including absence of modifications. Thus, for example, two unmodified DNA nucleoside have “the same type of modification,” even though the DNA nucleoside is unmodified. Such nucleosides having the same type modification may comprise different nucleobases.

The phrase “pharmaceutically acceptable carrier or diluent” refers to any substance suitable for use in administering to an animal. In certain embodiments, a pharmaceutically acceptable carrier or diluent is sterile saline. In certain embodiments, such sterile saline is pharmaceutical grade saline.

The terms “substituent” and “substituent group” refer to an atom or group that replaces the atom or group of a named parent compound. For example, a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, compounds of the present invention have substituents at one or at more than one position of the parent compound. Substituents may also be further substituted with other substituent groups and may be attached directly or via a linking group such as an alkyl or hydrocarbyl group to a parent compound.

When used in reference to a chemical functional group, the term “substituent” refers to an atom or group of atoms that differs from the atom or group of atoms normally present in the named functional group. In certain embodiments, a substituent replaces a hydrogen atom of the functional group (e.g., in certain embodiments, the substituent of a substituted methyl group is an atom or group other than hydrogen which replaces one of the hydrogen atoms of an unsubstituted methyl group). Unless otherwise indicated, groups amenable for use as substituents include without limitation, halogen, hydroxyl, alkyl, alkenyl, alkynyl, acyl (—C(O)Raa), carboxyl (—C(O)O—Raa), aliphatic groups, alicyclic groups, alkoxy, substituted oxy (—O—Raa), aryl, aralkyl, heterocyclic radical, heteroaryl, heteroarylalkyl, amino (—N(Rbb)(Rcc)), imino (═NRbb), amido (—C(O)N(Rbb)(Rcc) or —N(Rbb)C(O)Raa), azido (—N₃), nitro (—NO₂), cyano (—CN), carbamido (—OC(O)N(Rbb)(Rcc) or —N(Rbb)C(O)ORaa), ureido (—N(Rbb)C(O)N(Rbb)(Rcc)), thioureido (—N(Rbb)C(S)N(Rbb)-(Rcc)), guanidinyl (—N(Rbb)C(═NRbb)N(Rbb)(Rcc)), amidinyl (—C(═NRbb)N(Rbb)(Rcc) or —N(Rbb)C(═NRbbb)(Raa)), thiol (—SRbb), sulfinyl (—S(O)Rbb), sulfonyl (—S(O)₂Rbb) and sulfonamidyl (—S(O)₂N(Rbb)(Rcc) or —N(Rbb)S—(O)₂Rbb), wherein each Raa, Rbb and Rcc is, independently, H, an optionally linked chemical functional group or a further substituent group with a preferred list including without limitation, alkyl, alkenyl, alkynyl, aliphatic, alkoxy, acyl, aryl, aralkyl, heteroaryl, alicyclic, heterocyclic and heteroarylalkyl. Selected substituents within the compounds described herein are present to a recursive degree.

The term “alkyl” refers to a saturated straight or branched hydrocarbon radical containing up to 24 carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C1-C12 alkyl) with from 1 to about 6 carbon atoms being more preferred.

The term “alkenyl” refers to a straight or branched hydrocarbon chain radical containing up to 24 carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

The term “alkynyl” refers to a straight or branched hydrocarbon radical containing up to 24 carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

The term “acyl” refers to a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

The term “alicyclic” refers to a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

The term “aliphatic” refers to a straight or branched hydrocarbon radical containing up to 24 carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation, polyalkoxys, such as polyalkylene glycols, polyamines, and polyimines Aliphatic groups as used herein may optionally include further substituent groups.

The term “alkoxy” refers to a radical formed between an alkyl group and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

The term “aminoalkyl” refers to an amino substituted C1-C12 alkyl radical. The alkyl portion of the radical forms a covalent bond with a parent molecule. The amino group can be located at any position and the aminoalkyl group can be substituted with a further substituent group at the alkyl and/or amino portions.

The term “aralkyl” and “arylalkyl” mean an aromatic group that is covalently linked to a C1-C12 alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

The term “aryl” and “aromatic” mean a mono- or polycyclic carbocyclic ring system radicals having one or more aromatic rings. Examples of aryl groups include without limitation, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

The term “halo” and “halogen,” mean an atom selected from fluorine, chlorine, bromine and iodine.

The term “heteroaryl,” and “heteroaromatic,” mean a radical comprising a mono- or poly-cyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

The compounds described herein include variations in which one or more atoms are replaced with a non-radioactive isotope or radioactive isotope of the indicated element. For example, compounds herein that comprise hydrogen atoms encompass all possible deuterium substitutions for each of the 1H hydrogen atoms.

Isotopic substitutions encompassed by the compounds herein include but are not limited to e.g., ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S, etc. In certain embodiments, non-radioactive isotopic substitutions may impart new properties on the oligomeric compound that are beneficial for use as a therapeutic or research tool. In certain embodiments, radioactive isotopic substitutions may make the compound suitable for research purposes such as imaging.

As used herein, the term “nanoparticle” refers to any particle having an average diameter of less than 500 nanometers (nm). In some embodiments, nanoparticles have an average diameter of less than 300 nm, less than 100 nm, less than 50 nm, less than 25 nm, less than 10 nm or less than 5 nm. In some embodiments, each nanoparticle has a diameter of less than 300 nm, less than 100 nm, less than 50 nm, less than 25 nm, less than 10 nm or less than 5 nm.

As used herein, the term “hypertrophy” refers to an increase in mass of an organ or structure independent of natural growth that does not involve tumor formation. Hypertrophy of an organ or tissue is due either to an increase in the mass of the individual cells (true hypertrophy), or to an increase in the number of cells making up the tissue (hyperplasia), or both. Certain organs, such as the heart, lose the ability to divide shortly after birth.

The term “fibrosis-related genes” refers to genes that encode proteins involved in the fibrosis process, including proteins that participate the fibrosis process directly, such as extracellular matrix proteins, and proteins that modulate the fibrosis process, such as GATA4 and eIF4G2 proteins. Fibrosis-related genes include pro-fibrosis genes and anti-fibrosis genes. Expression of a pro-fibrosis gene facilitates fibrosis, while expression of an anti-fibrosis gene inhibits fibrosis.

The term “cardiac fibrosis-related genes” refers to fibrosis-related genes that are involved in the fibrosis process in the heart. Example of cardiac fibrosis-related genes include, but not limited to, pro-fibrosis genes such as eukaryotic translation initiation factor 4 gamma 2 (eIF4G2), glutamyl-prolyl-tRNA synthetase (EPRS) and mesenchyme Homeobox 1 (MEOX1) and anti-fibrosis genes such as GATA binding protein 4 (GATA4), myocyte enhancer factor 2C (MEF2C), NK2 homeobox 5 (NKX2-5), T-box transcription factor 5 (TBX5), hepatocyte nuclear factor 4 alpha (HNF4a), alpha crystalline B (CRYAB), transcription factor 21 (TCF21) and myosin binding protein C (MYBPC3).

As used herein, the term “treating,” “treatment,” and the like relate to any treatment of cardiac fibrosis, including but not limited to prophylactic treatment and therapeutic treatment. “Treating” includes any effect, e.g., lessening, reducing, modulating, or eliminating, that results in the improvement of the cardiac fibrosis. “Treating” or “treatment” of cardiac cardiac fibrosis includes inhibiting the inhibiting the cardiac cardiac fibrosis, i.e., arresting the development of the cardiac cardiac fibrosis or its clinical symptoms; or relieving the cardiac cardiac fibrosis, i.e., causing temporary or permanent regression of the disease or its clinical symptoms. Those in need of treatment include those already with cardiac fibrosis and those in whom cardiac fibrosis is to be prevented.

As used herein, the term “subject” refers to a mammal, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). A “subject in need thereof” refers to a subject who may have, is diagnosed with, is suspected of having, or requires prevention of cardiac fibrosis.

An “effective amount” or a “therapeutically effective amount” is defined herein in relation to the treatment of cardiac fibrosis is an amount that when administered alone or in combination with another therapeutic agent to a cell, tissue, or subject is effective to decrease, reduce, inhibit, or otherwise abrogate the growth of the cardiac fibrosis. An “effective amount” further refers to that amount of the compound sufficient to result in amelioration of symptoms, e.g., treatment, healing, prevention, or amelioration for the cardiac fibrosis, or in increase in the rate of treatment, healing, prevention, or amelioration of cardiac hypertrophy. When applied to an individual compound (active ingredient) administered alone, an “effective amount” refers to that ingredient alone. When applied to a combination, the “effective amount” refers to combined amounts of the active ingredients that result in the therapeutic effect, whether administered in combination, serially, or simultaneously. The “effective amount” will vary depending the cause of the cardiac fibrosis and the severity of the cardiac fibrosis, as well as the age, weight, etc., of the subject to be treated. Additionally, the “effective amount” can vary depending upon the dosage form employed and the route of administration utilized. A physician or veterinarian having ordinary skill in the art can readily determine and prescribe the effective amount (e.g., ED50) of the active ingredients required. For example, the physician or veterinarian can start doses of the administered compounds at levels lower than that required in order to achieve the desired therapeutic effect and gradually increase the dosage until the desired effect is achieved.

### II. Compositions for Modulating Translation

Translation of a protein encoded by a messenger ribonucleic acid (mRNA) usually begins at the start codon of the main open reading frame (mORF) of the mRNA. Some mRNAs contain one or more upstream ORFs (uORFs) located in the 5′ untranslated region of mRNAs. uORFs have been established as a negative regulatory element to repress the translation of mORFs when their corresponding uORF is translated. Antisense oligonucleotide (ASO) technology provides an effective means for modulating the expression of specific mRNAs or proteins based on Watson-Crick base-pairing between an appropriately designed ASO and its target mRNA. ASO technology has been used most often to reduce the amount an mRNA via antisense-induced RNase H cleavage or to alter splicing of a pre-mRNA transcript in a cell. In contrast, the present application provides antisense oligonucleotides (ASOs) and modified antisense oligonucleotides that are not primarily designed to elicit cleavage. Specifically, the present application provides antisense oligonucleotides and modified oligonucleotides that can selectively increase or decrease translation of a desired target protein in a cell by disrupting a double stranded region in an uORF of an mRNA (hereinafter referred to as Type I ASO or ASO1), or forming a intermolecular double stranded region that is downstream of, and adjacent to, a uORF start codon or a mORF start codon (hereinafter generally referred to as Type II ASO or ASO2).

The present application also provides antisense oligonucleotides and modified oligonucleotides that have a gapmer structure capable of forming a double stranded region with sequences in the 5′-UTR region of an mRNA and decreasing translation of a desired target protein and degrading the target mRNA in a cell by 5′ UTR-targeting gapmer ASOs.

The term “gapmer” refers to a chimeric oligomeric compound comprising a central region (a “gap”) and a region on either side of the central region (the “wings”), wherein the gap comprises at least one modification difference compared to each wing. Such modifications include nucleobase, monomeric linkage, and sugar modifications as well as the absence of modification (unmodified RNA or DNA). Thus, in certain embodiments, the nucleotide linkages in each of the wings are different than the nucleotide linkages in the gap. In certain embodiments, each wing comprises nucleotides with high affinity modifications and the gap comprises nucleotides that do not comprise that modification. In certain embodiments the nucleotides in the gap and the nucleotides in the wings all comprise high affinity modifications, but the high affinity modifications in the gap are different than the high affinity modifications in each of the wings. In certain embodiments, the modifications in the wings are the same as one another. In certain embodiments, the modifications in the wings are different from each other. In certain embodiments, nucleotides in the gap are unmodified and nucleotides in the wings are modified. In certain embodiments, the modification(s) within each wing are the same. In certain embodiments, the modification(s) in one wing are different from the modification(s) in the other wing. Gapmers have been described in U.S. Pat. Nos. 9,550,988 and 9,045,754, which are incorporated herein by reference.

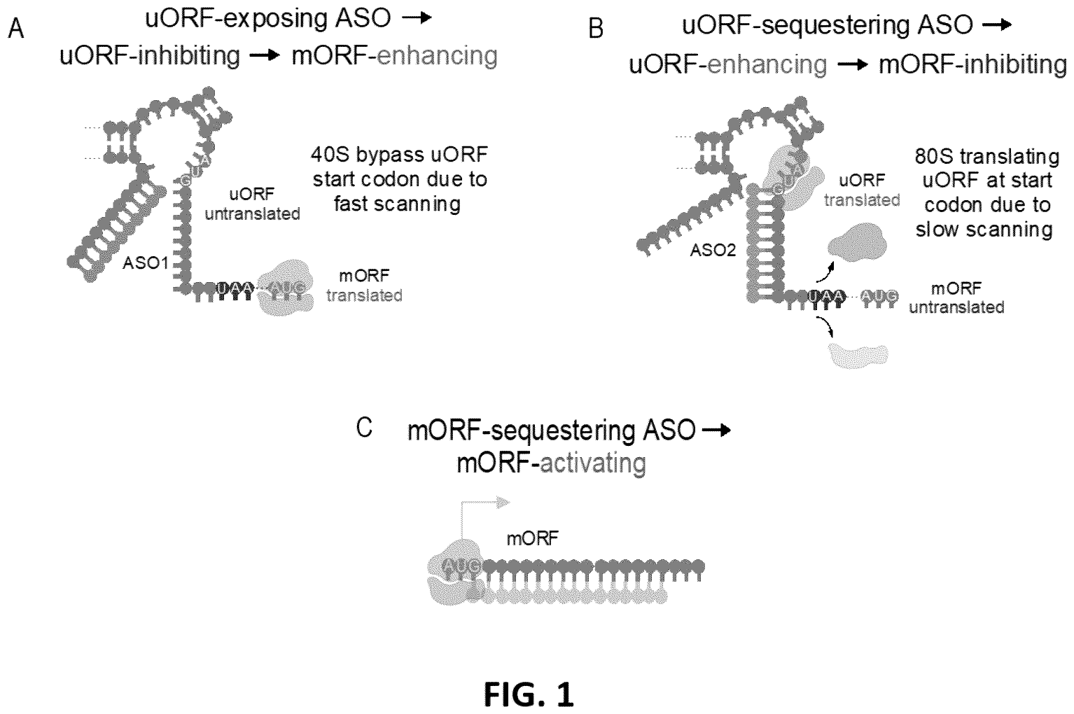

Although not wishing to be bound by theory, it is the inventor's hypothesis that type I ASOs (e.g., ASO1) disrupt the original double-stranded structure in the uORF by forming a double-stranded structure with the non-coding strand of the original double-stranded structure, thus inhibiting translation of the uORF, which in turn results in enhanced translation of the corresponding mORF (FIG. 1, Panel A).

In contrast, type II ASOs disrupts the original double-stranded structure in the uORF by forming a double-stranded structure with the coding strand of the original double-stranded structure, thus enhancing translation of the uORF, which in turn results in reduced translation of the corresponding mORF (FIG. 1, Panel B). Furthermore, type II ASOs may function in the absence of the original double-stranded structure in the uORF. In this case, type II ASOs may be designed to form a double-stranded structure with a target sequence downstream of, and adjacent to, the start codon of a uORF, forming a double-stranded structure with the target sequence downstream of the uORF start codon, thus enhancing translation of the uORF, which in turn results in reduced translation of the corresponding mORF. These uORF-targeting type II ASOs are referred to as “type II uotASOs”.

In addition, type II ASOs may be designed to form a double-stranded structure with a target sequence downstream of, and adjacent to, the start codon of a mORF, forming a double-stranded structure with the target sequence downstream of the mORF start codon, thus enhancing translation of the mORF. These mORF-targeting type II ASOs are referred to as “type II motASOs”.

The 5′ UTR-targeting Gapmer ASOs may be designed to form a double-stranded structure with a target sequence in the 5′-UTR of a mRNA of a target gene. In some embodiments, the target sequence is located in a region that spans from 55 nucleotides upstream of an uORF start codon to 55 nucleotides downstream of the uORF start codon of the mRNA, thus enhancing translation of the uORF, or blocking the scanning of pre-initiation complex, which in turn results in reduced translation of the downstream mORF, and alternatively trigger RNase H1-mediated mRNA degradation.

### ASOs Enhancing Expression of Anti-Fibrosis Gene Products

One aspect of the present application is directed to antisense oligonucleotides (ASOs) that is capable of selectively increase or decrease translation of a cardiac fibrosis-related gene product. The design and use of these ASOs can be utilized for treatment or prevention of cardiac fibrosis.

In some embodiments, the antisense oligonucleotide (ASO) of the present application is capable of binding to, and forming a double-stranded structure with, a target sequence located in a non-coding strand of a double-stranded stem structure downstream of, and adjacent to, a uORF AUG start codon of a mRNA of a cardiac fibrosis-related gene. The binding of the ASO disrupts the double-stranded stem structure of the uORF and enhances translation of the corresponding mORF of the cardiac fibrosis-related gene. (Type I uotASO)

In some embodiments, the cardiac fibrosis-related gene is an anti-fibrosis gene. Examples of anti-fibrosis gene include, but are not limited to, GATA4, MEF2C, NKX2-5, TBX5, HNF4A, CRYAB, TCF21 and MYBCP3.

In some embodiments, the target sequence comprises human GATA4 mRNA sequence of SEQ ID NO:27.

In some embodiments, the ASO comprises a sequence that is at least 500%, 60%, 70%, 80% or 90% complementary to the target sequence. In some embodiments, the ASO comprises a sequence that is 100% complementary to the target sequence. In some embodiments, the ASO further comprises one or more modified nucleotide and/or modified internucleotide linkage.

In some embodiments, the ASO is a human GATA4 type I uotASO comprising SEQ ID NO:8.

In some embodiments, the ASO is capable of forming a double-stranded structure with a target sequence downstream of, and adjacent to, a mORF start codon of the mRNA of an anti-fibrosis gene and enhances mORF translation of the anti-fibrosis gene (type II motASO).

In some embodiments, the target region includes regions that are two to eight nucleotides away from the adenine (A) of the mORF AUG start codon.

In some embodiments, the anti-fibrosis gene is GATA4, MEF2C, NKX2-5, TBX5, HNF4A, CRYAB, TCF21 and MYBPC3. In particular embodiments, the anti-fibrosis gene is MYBPC3 or CRYAB. An overview of MYBPC3 and CRYAB and their indications is shown in the table below. Suitable targets are downregulated in relevant human tissues in cardiac metabolism diseases and where an upregulation is considered therapeutically favorable.

| | | | | Disease |
|---|---|---|---|---|
| Target | Function | Target Cell | Indication | Association |
| MYBCP3 | Structural | Cardiac | HCM | Impaired |
| Myosin- | protein crucial | myocyte | Cardiomyopathy | function leads to |
| binding | for sarcomere | | (haploinsufficiency) | severe cardiac |
| protein C, | organization | | | hypertrophy and |
| cardiac-type | | | | interstitial |
| | | | | fibrosis. >50 |
| | | | | mutations |
| | | | | in MYBPC3 have |
| | | | | been identified. |
| CRYAB | Heat shock | Cardiac | HFrEF, DCM | Human genetic |
| Alpha- | protein / | myocyte | Cardiomyopathy | association with |
| crystallin B | molecular | | (haploinsufficiency) | dilated |
| chain | chaperone | | | cardiomyopathy |
| | | | | and myofibrillar |
| | | | | myopathy. |

In some embodiments, the target sequence comprises the nucleotide sequence 5′-gcctgagccggggaag-3′ human MYBPC3 type II motASO target sequence, SEQ ID NO:47.

In some embodiments, the target sequence comprises the nucleotide sequence 5′-ggacatcgccatccac-3′ human CRYAB type II motASO target sequence, SEQ ID NO:48

In some embodiments, the target sequence comprises human GATA4 mRNA sequence of SEQ ID NO:28.

In some embodiments, the target sequence comprises human MEF2C mRNA sequence of SEQ ID NO:29.

In some embodiments, the target sequence comprises human NKX2-5 mRNA sequence of SEQ ID NO:30.

In some embodiments, the ASO comprises a sequence that is at least 50%, 60%, 70%, 80% or 90% complementary to the target sequence. In some embodiments, the ASO comprises a sequence that is 100% complementary to the target sequence. In some embodiments, the ASO further comprises one or more modified nucleotide and/or modified internucleotide linkage.

In some embodiments, the ASO is a human MYBPC3 type II motASO comprising SEQ ID NO:45.

In some embodiments, the ASO is a human CRYAB type II motASO comprising SEQ ID NO:46.

In some embodiments, the ASO is a human GATA4 type II motASO comprising SEQ ID NO:9 or SEQ ID NO: 10.

In some embodiments, the ASO is a human NKX2-5 type II motASO comprising SEQ ID NO:15.

In some embodiments, the ASO is a human MEF2C type II motASO comprising SEQ ID NO:21.

In some embodiments, the 5′ end of the ASO binds to a nucleotide position between +3 to +19 relative to the AUG start codon of the mORF, where +1 corresponds to the adenine in the AUG start codon. In some embodiments, the 3′ end of the ASO includes at least one nucleotide complementary to a nucleotide within the mORF start codon. In certain embodiments, the 3′ end of the ASO includes a cytosine, which is complementary to the guanine in the AUG start codon.

### ASOs Reducing Expression of Pro-Fibrosis Gene Products

In some embodiments, the ASO is capable of forming a double-stranded structure with a target sequence downstream of, and adjacent to, a start codon of a uORF in the mRNA of a pro-fibrosis gene and inhibits mORF translation of the pro-fibrosis gene. (type II uotASO) In some embodiments, the target region includes regions that are two to eight nucleotides away from the adenine (A) of the uORF AUG start codon.

In some embodiments, the pro-fibrosis gene is eIF4G2, EPRS or MEOX1.

In some embodiments, the pro-fibrosis gene is eIF4G2, which is an essential fibrotic-stress mediator for extracellular matrix (ECM) mRNAs translation. Genetic knockout of eIF4G2 in cardiac myofibroblasts (Postn^{MCM}) attenuates cardiac dysfunction, pathological hypertrophy and fibrosis. The TGFβ-eIF4G2-IGFBP7 axis is a novel translation regulatory pathway mediating cardiac fibroblast activation and plays a key role in cardiac fibrosis. It has been shown that genetic knockout of eIF4G2 in cardiomyocytes (Myh6^{MCM}) does not cause severe heart disease within 5 months.

In some embodiments, the target sequence comprises human EIF4G2 mRNA sequence of SEQ ID NO:31.

In some embodiments, the ASO comprises a sequence that is at least 50%, 60%, 70%, 80% or 90% complementary to the target sequence. In some embodiments, the ASO comprises a sequence that is 100% complementary to the target sequence. In some embodiments, the ASO further comprise one or more modified nucleotide and/or modified internucleotide linkage.

In some embodiments, the 5′ end of the ASO binds to a nucleotide position between +3 to +19 relative to the AUG start codon of the uORF, where +1 corresponds to the adenine in the AUG start codon. In some embodiments, the 3′ end of the ASO includes at least one nucleotide complementary to a nucleotide within the uORF start codon. In certain embodiments, the 3′ end of the ASO includes a cytosine, which is complementary to the guanine in the AUG start codon.

In some embodiments, the ASO is a human EIF4G2 type II uotASO comprising SEQ ID NO: 17.

In some embodiments, the ASO is a 5′-UTR-targeting gapmer ASO that targets the 5′-UTR of the mRNA of a pro-fibrosis gene. In some embodiments, 5′-UTR-targeting gapmer ASO is capable of binding to a target located in a region that spans from 55 nucleotides upstream to 55 nucleotides downstream of an uORF start codon in the mRNA. In some embodiments, 5′-UTR-targeting gapmer ASO is capable of binding to a target located in a region that spans from 45 nucleotides upstream to 45 nucleotides downstream of an uORF start codon in the mRNA. In some embodiments, 5′-UTR-targeting gapmer ASO is capable of binding to a target located in a region that spans from 35 nucleotides upstream to 35 nucleotides downstream of an uORF start codon in the mRNA. In some embodiments, 5′-UTR-targeting gapmer ASO is capable of binding to a target located in a region that spans from 25 nucleotides upstream to 25 nucleotides downstream of an uORF start codon in the mRNA. In some embodiments, 5′-UTR-targeting gapmer ASO is capable of binding to a target located in a region that spans from 15 nucleotides upstream to 15 nucleotides downstream of an uORF start codon in the mRNA.

In some embodiments, the 5′-UTR-targeting gapmer ASO comprises a sequence that is at least 50%, 60%, 70%, 80% or 90% complementary to the target sequence. In some embodiments, the 5′-UTR-targeting gapmer ASO comprises a sequence that is 100% complementary to the target sequence.

In some embodiments, the 5′-UTR-targeting gapmer ASO targets the 5′-UTR of the mRNA of human eIF4G2 gene. In some embodiments, the 5′-UTR-targeting gapmer ASO has a gap region of 5-20 nucleotides, flanked by two wing regions of 3-10 nucleotides. In some embodiments, the 5′-UTR-targeting gapmer ASO has a gap region of 10 nucleotides, flanked by two wing regions of 5 nucleotides.

In some embodiments, the target sequence comprises human eIF4G2 mRNA sequence of SEQ ID NO:49.

In some embodiments, the 5′-UTR-targeting gapmer ASO having a nucleotide sequence of SEQ ID NO:41.

### ASO Design Procedure

Step 1. Determine the dominant alternative spliced mRNA isoform in the organ as ASO target.

Step 2. Examine multiple parameters for ASO design, including 5′ UTR length, uORF presence, 5′ UTR GC content, dsRNA element, KOZAK sequence around uORF and mORF start codons, and effects from dsRNA-binding protein or RNA helicase.

Step 3. Mechanism-based screen of ASOs: (1) Target uORF start codon using Type I uotASO that inhibits uORF while enhancing mORF translation; (2) Target mORF start codon using Class II motASO that directly enhances mORF translation.

Step 4 (Target-to-Hit). Perform a tiling screen by shifting the ASO from initial ASO to target 3-nt (and then 1-nt) upstream or downstream regions.

Step 5 (Hit-to-Lead). Optimal ASO identified for in vivo testing in animal model after validation of ASO effects in human cell lines and primary mouse cells.

Exemplary embodiments of design strategies and sequences for MYBPC3 and CRYAB targets are shown in the below table for mORF-activating Type II motASO.

| | | | | | Translation |
|---|---|---|---|---|---|
| Target mRNA | Protein function | uORF | 5′-UTR | Lead ASO | effect |
| MYBPC3 (CM) | A CM-specific | No | 55-nt 5′- | CmoUmoUmoC | Activation |
| myosin-binding | structural | | UTR; G-, U-, C-rich; | moCmoCmoCm | |
| protein C | myofilament | | contain weak | oGmoGmoCmo | |
| | protein. | | Kozak | UmoCmoAmoG | |
| | Heterozygous | | sequence in | moGmoCm | |
| | human LOF | | mORF | (SEQ ID NO: 45) | |
| | mutations (e.g., | | | | |
| | nonsense | | | | |
| | mutations) lead to | | | | |
| | haploinsufficiency | | | | |
| | and cause | | | | |
| | hypertrophic | | | | |
| | cardiomyopathy | | | | |
| CRYAB (CM) | Heat shock | Yes | 273-nt 5′- | GmoUmoGmoG | Activation |
| crystallin | protein that can | | UTR; contain | moAmoUmoGm | |
| alpha B | protect desmin | | very strong | oGmoCmoGmo | |
| | protein and CM | | Kozak | AmoUmoGmoU | |
| | cytoskeleton | | sequence in | moCmoCm | |
| | network. | | mORF | (SEQ ID NO: 46) | |
| | Heterozygous | | | | |
| | human LOF | | | | |
| | mutations lead to | | | | |
| | dilated | | | | |
| | cardiomyopathy. | | | | |

In some embodiments, the ASOs of the present application (e.g., Type I uotASOs, Type II uotASOs, Type II motASOs and 5′-UTR-targeting gapmer ASOs) have a length between 8 to 50, 8 to 40, 8 to 30, 8 to 25, 8 to 20, 8 to 16, 8 to 12, 10 to 50, 10 to 40, 10 to 30, 10 to 25, 10 to 20, 10 to 16, 10 to 12, 12 to 50, 12 to 40, 12 to 30, 12 to 25, 12 to 20, 12 to 16, 15 to 50, 15 to 40, 15 to 30, 15 to 25, 15 to 20, 20 to 50, 20 to 40, 20 to 30, or 20 to 25 nucleotides.

### Modified Nucleosides

ASOs of the present application may comprise or consist of oligonucleotides comprising at least one modified nucleoside. Such modified nucleosides may comprise a modified sugar moiety, a modified nucleobase, or both. In some embodiments, the ASO comprises at least 5, at least 10, at least 15, at least 20, at least 25 or more modified nucleosides relative to the total number of nucleosides in the ASO. In some embodiments, the modified ASO includes a modified region of at least 5, at least 10, at least 15, at least 20, at least 25 or more contiguous modified nucleosides in the ASO. In some embodiments, each of the nucleosides in the ASO is modified.

In certain preferred embodiments, the one or more modified nucleotides include a 2′-O-methyl modified sugar moiety and/or a modified internucleoside linkage. In some embodiments, the modified internucleoside linkage is a phosphodiester internucleoside linkage or a phosphorothioate internucleoside linkage.

In some embodiments, the ASO of the present application comprises one or more sugar-modified nucleotides. In some embodiments, the ASO comprises the nucleotide sequence of any one of SEQ ID NOs:3-6 with one or more modified sugar moieties and/or modified internucleoside linkages.

In certain particular embodiments, the ASO comprises the nucleotide sequence of AmoCmoGmoUmoAmoUmoUmoAmoAmoAmoUmoCmoCmoAmoGmoCm (SEQ ID NO:7), or AmoCmoGmoAmoAmoUmoUmoAmoAmoAmoUmoCmoCmoAmoGmoCm (SEQ ID NO:8), CmoUmoUmoCmoCmoCmoCmoGmoGmoCmoUmoCmoAmoGmoGmoCm (SEQ ID NO:45) or GmoUmoGmoGmoAmoUmoGmoGmoCmoGmoAmoUmoGmoUmoCmoCm (SEQ ID NO:46) where “m” indicates a 2′-O-methyl modification, and “o” indicates a phosphodiester or phosphorothioate internucleoside linkage. It should be noted that in any of the sequences disclosed in the present application, where the modifications “o” or “mo” are included, such modifications may be substituted with any nucleoside modifications described herein or they may contain no nucleoside modifications at all.

In certain particular embodiments, the ASO comprises the nucleotide sequence of GesCesCesAesCesCdsTdsCdsCdsAdsTdsAdsGdsAdsGdsCesUesCesCesGe (SEQ ID NPO:41), wherein “e” indicates 2′-O-methoxyethyl (MOE) modification, “s” indicates a phosphorothioate internucleoside linkage, and “d” indicates DNA.

### Sugar Moieties

The ASOs of the present application may contain nucleosides with naturally occurring sugar moieties and/or nucleosides with modified sugar moieties. ASOs comprising nucleosides with modified sugar moieties may have desirable properties, such as enhanced nuclease stability or increased binding affinity with a target nucleic acid relative to ASOs comprising only nucleosides comprising naturally occurring sugar moieties. In some embodiments, the modified sugar moieties are substituted sugar moieties. In certain embodiments, the modified sugar moieties are bicyclic or tricyclic sugar moieties. In certain embodiments, the modified sugar moieties are sugar surrogates. Such sugar surrogates may include one or more substitutions corresponding to those of substituted sugar moieties.

In certain embodiments, the modified sugar moieties are substituted sugar moieties comprising one or more substituent, including but not limited to substituents at the 2′ and/or 5′ positions. Examples of sugar substituents suitable for the 2′-position, include, but are not limited to: 2′-F, 2′-OCH₃ (“O-methyl”), and 2′-O(CH₂)₂OCH₃. In certain embodiments, sugar substituents at the 2′ position is selected from allyl, amino, azido, thio, O-allyl, O-C1-C10 alkyl, O-C1-C10 substituted alkyl; O-C1-C10 alkoxy; O-C1-C10 substituted alkoxy, OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C1-C10 alkyl. Examples of sugar substituents at the 5′-position, include, but are not limited to: 5′-methyl (R or S); 5′-vinyl, and 5′-methoxy. In certain embodiments, substituted sugars comprise more than one non-bridging sugar substituent, for example, 2′-F-5′-methyl sugar moieties (see, e.g., PCT International Application WO 2008/101157, for additional 5′,2′-bis substituted sugar moieties and nucleosides).

Nucleosides comprising 2′-substituted sugar moieties are referred to as 2′-substituted nucleosides. In certain embodiments, a 2′-substituted nucleoside comprises a 2′-substituent group selected from halo, allyl, amino, azido, O-C1-C10 alkoxy; O-C1-C10 substituted alkoxy, SH, CN, OCN, CF₃, OCF₃, O-alkyl, S-alkyl, N(Rm)-alkyl; O-alkenyl, S-alkenyl, or N(Rm)-alkenyl; O-alkynyl, S-alkynyl, N(Rm)-alkynyl; O-alkylenyl-O-alkyl, alkynyl, alkaryl, aralkyl, O-alkaryl, O-aralkyl, O(CH₂)₂SCH₃, O—(CH₂)₂—O—N(Rm)(Rn) or O—CH₂—C(═O)—N(Rm)(Rn), where each Rm and Rn is, independently, H, an amino protecting group or substituted or unsubstituted C1-C10 alkyl. These 2′-substituent groups can be further substituted with one or more substituent groups independently selected from hydroxyl, amino, alkoxy, carboxy, benzyl, phenyl, nitro (N02), thiol, thioalkoxy (S-alkyl), halogen, alkyl, aryl, alkenyl and alkynyl.

In certain embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rm)(Rn), O(CH₂)₂O(CH₂)₂N(CH₃)₂, and N-substituted acetamide (O—CH₂—C(═O)—N(Rm)(Rn) where each Rm and Rn is, independently, H, an amino protecting group or substituted or unsubstituted C1-C10 alkyl.

In certain embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, O(CH₂)₂O(CH₂)₂N(CH₃)₂, and O—CH₂—C(═O)—N(H)CH₃.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ sugar substituents, include, but are not limited to: —[C(Ra)(Rb)]n-, —[C(Ra)(Rb)]n-O—, —C(RaRb)—N(R)—O— or, C(RaRb)—O—N(R)—; 4′-CH₂-2′, 4′-(CH2)₂-2′, 4′—(CH₂)₃-2′, 4′—(CH₂)—O-2′ (LNA); 4′-(CH₂)—S-2; 4′-(CH₂)₂—O-2′ (ENA); 4′-CH(CH₃)—O-2′ (cEt) and 4′-CH(CH₂OCH₃)—O-2′, and analogs thereof (see, e.g., U.S. Pat. No. 7,399,845, issued on Jul. 15, 2008); 4′-C(CH₃)(CH₃)—O-2′ and analogs thereof, (see, e.g., WO2009/006478, published Jan. 8, 2009); 4′-CH₂—N(OCH₃)-2′ and analogs thereof (see, e.g., WO2008/150729, published Dec. 11, 2008); 4′-CH₂—O—N(CH₃)-2′ (see, e.g., US2004/0171570, published Sep. 2, 2004); 4′-CH₂—O—N(R)-2′, and 4′-CH₂—N(R)—O-2′-, wherein each R is, independently, H, a protecting group, or C1-C12 alkyl; 4′-CH₂—N(R)—O-2′, wherein R is H, C1-C12 alkyl, or a protecting group (see, U.S. Pat. No. 7,427,672, issued on Sep. 23, 2008); 4′-CH₂—C(H)(CH₃)-2′ (see, e.g., Chattopadhyaya, et al., J. Org. Chem., 2009, 74, 118-134); and 4′-CH₂—C(═CH₂)-2′ and analogs thereof (see, published PCT International Application WO 2008/154401, published on Dec. 8, 2008).

In certain embodiments, such 4′ to 2′ bridges independently comprise from 1 to 4 linked groups independently selected from —[C(Ra)(Rb)]n-, —C(Ra)═C(Rb)—, —C(Ra)=N—, —C(═NRa)—, —C(═O)—, —C(═S)—, —O—, —Si(Ra)₂—, —S(═O)x-, and —N(Ra)—;
wherein:
x is 0, 1, or 2;
n is 1, 2, 3, or 4;
each Ra and Rb is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O) H), substituted acyl, a heterocycle radical, a substituted heterocycle radical, C1-C12 aminoalkyl, substituted C1-C12 aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂ O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio(4′—CH₂—S-2′) BNA, (H) methylene-amino (4′-CH₂—N(R)-2′) BNA, (I) methyl carbocyclic (4′-CH₂CH(CH₃)-2′) BNA, (7) propylene carbocyclic (4′-(CH₂)₃-2′) BNA, and (M) 4′-CH₂—O—CH₂-2′ as depicted below.

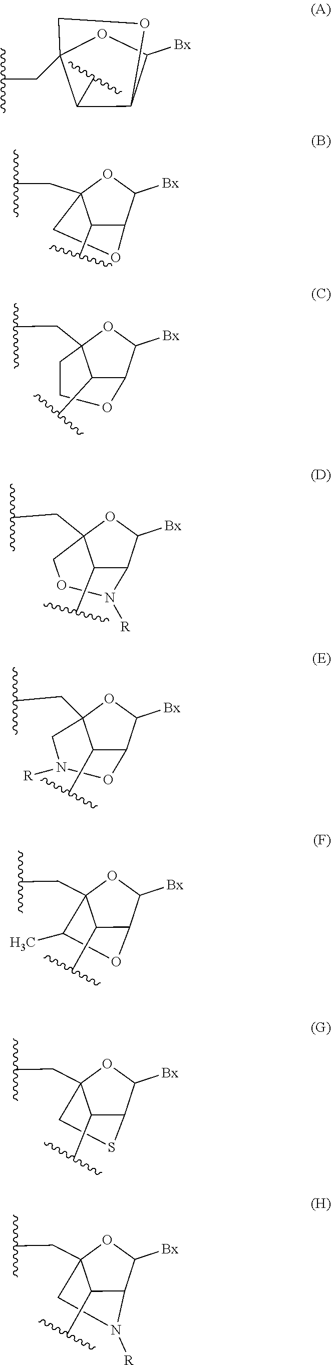

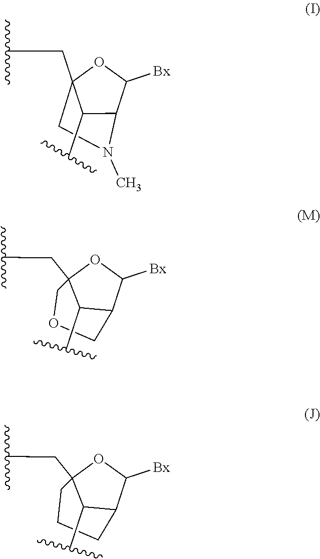

wherein Bx is a nucleobase moiety and R is, independently, H, a protecting group, or C1-C12 alkyl.

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US2004/0171570, US2007/0287831, and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In certain embodiments, bicyclic sugar moieties and nucleosides incorporating such bicyclic sugar moieties are further defined by isomeric configuration. For example, a nucleoside comprising a 4′-2′ methylene-oxy bridge, may be in the α-L configuration or in the β-D configuration. Previously, α-L-methyleneoxy (4′-CH2-O-2′) bicyclic nucleosides have been incorporated into antisense oligonucleotides that showed antisense activity (Frieden et al., Nucleic Acids Research, 2003, 21, 6365-6372).

In certain embodiments, substituted sugar moieties comprise one or more non-bridging sugar substituent and one or more bridging sugar substituent (e.g., 5′-substituted and 4′-2′ bridged sugars). (see, PCT International Application WO 2007/134181, published on Nov. 22, 2007, wherein LNA is substituted with, for example, a 5′-methyl or a 5′-vinyl group).

In certain embodiments, modified sugar moieties are sugar surrogates. In certain such embodiments, the oxygen atom of the naturally occurring sugar is substituted, e.g., with a sulfer, carbon or nitrogen atom. In certain such embodiments, such modified sugar moiety also comprises bridging and/or non-bridging substituents as described above. For example, certain sugar surrogates comprise a 4′-sulfer atom and a substitution at the 2′-position (see, e.g., published U.S. Patent Application US2005/0130923, published on Jun. 16, 2005) and/or the 5′ position. By way of additional example, carbocyclic bicyclic nucleosides having a 4′-2′ bridge have been described (see, e.g., Freier et al., Nucleic Acids Research, 1997, 25(22), 4429-4443 and Albaek et al., J. Org. Chem., 2006, 71, 7731-7740).

In certain embodiments, sugar surrogates comprise rings having other than 5-atoms. For example, in certain embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), 4amanti nucleic acid (MNA) (see Leumann, C J. Bioorg. & Med. Chem. (2002) 10:841-854), fluoro HNA (F-HNA), and those compounds having Formula VII:

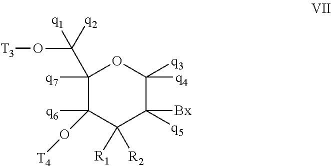

wherein independently for each of said at least one tetrahydropyran nucleoside analog of Formula VII:
Bx is a nucleobase moiety;
T3 and T4 are each, independently, an internucleoside linking group linking the tetrahydropyran nucleoside analog to the antisense oligonucleotide or one of T3 and T4 is an internucleoside linking group linking the tetrahydropyran nucleoside analog to the antisense oligonucleotide and the other of T3 and T4 is H, a hydroxyl protecting group, a linked conjugate group, or a 5′ or 3′-terminal group;
q1, q2, q3, q4, q5, q6 and q7 are each, independently, H, C1-C6 alkyl, substituted C1-C6 alkyl, C2-C6 alkenyl, substituted C2-C6 alkenyl, C2-C6 alkynyl, or substituted C2-C6 alkynyl; and
each of R1 and R2 is independently selected from among: hydrogen, halogen, substituted or unsubstituted alkoxy, NJ1J2, SJ1, N3, OC(═X)J1, OC(═X)NJ1J2, NJ3C(═X)NJ1J2, and CN, wherein X is O, S or NJ1, and each J1, J2, and J3 is, independently, H or C1-C6 alkyl.

In certain embodiments, the modified THP nucleosides of Formula VII are provided wherein q1, q2, q3, q4, q5, q6 and q7 are each H. In certain embodiments, at least one of q1, q2, q3, q4, q5, q6 and q7 is other than H. In certain embodiments, at least one of q1, q2, q3, q4, q5, q6 and q7 is methyl. In certain embodiments, THP nucleosides of Formula VII are provided wherein one of R1 and R2 is F. In certain embodiments, R1 is fluoro and R2 is H, R1 is methoxy and R2 is H, and R1 is methoxyethoxy and R2 is H.

Many other bicyclic and tricyclic sugar and sugar surrogate ring systems are known in the art that can be used to modify nucleosides (see, e.g., review article: Leumann, J C, Bioorganic & Medicinal Chemistry, 2002, 10, 841-854).

In certain embodiments, sugar surrogates comprise rings having more than 5 atoms and more than one heteroatom. For example, nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following structure:

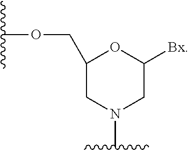

In certain embodiments, morpholinos may be modified, for example by adding or altering various substituent groups from the above morpholino structure. Such sugar surrogates are referred to herein as “modified morpholinos.”

Combinations of modifications are also provided without limitation, such as 2′-F-5′-methyl substituted nucleosides (see PCT International Application WO 2008/101157 Published on Aug. 21, 2008 for other disclosed 5′,2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

### Modified Nucleobases

In certain embodiments, nucleosides of the present invention comprise one or more unmodified nucleobases. In certain embodiments, nucleosides of the present invention comprise one or more modified nucleobases.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and 0-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, certain of which are commonly owned with the instant application, and each of which is herein incorporated by reference in its entirety.

### Internucleoside Linkages

In certain embodiments, nucleosides may be linked together using any internucleoside linkage to form oligonucleotides. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In certain embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or (S), α or β such as for sugar anomers, or as (D) or (L) such as for amino acids etc. Included in the antisense oligonucleotides provided herein are all such possible isomers, as well as their racemic and optically pure forms.

Neutral internucleoside linkages include without limitation, phosphotriesters, methylphosphonates, MMI (3′-CH₂—N(CH₃)O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### Motifs

In some embodiments, the ASO of the present application comprises a modified oligonucleotide. In some embodiments, the modified oligonucleotide comprises one or more modified sugars. In some embodiments, the modified oligonucleotide comprises one or more modified nucleobases. In some embodiments, the modified oligonucleotide comprises one or more modified internucleoside linkages. In some embodiments, the modifications (sugar modifications, nucleobase modifications, and/or linkage modifications) define a pattern or motif. In some embodiments, the patterns of chemical modifications of sugar moieties, internucleoside linkages, and nucleobases are each independent of one another. Thus, a modified oligonucleotide may be described by its sugar modification motif, internucleoside linkage motif and/or nucleobase modification motif (as used herein, nucleobase modification motif describes the chemical modifications to the nucleobases independent of the sequence of nucleobases).

In certain embodiments, every sugar moiety of the modified oligonucleotides of the present invention is modified. In certain embodiments, modified oligonucleotides include one or more unmodified sugar moiety.

### Overall Lengths

In certain embodiments, the present invention provides modified oligonucleotides of any of a variety of ranges of lengths. In certain embodiments, the invention provides oligomeric compounds or oligonucleotides consisting of X to Y linked nucleosides, where X represents the fewest number of nucleosides in the range and Y represents the largest number of nucleosides in the range. In certain such embodiments, X and Y are each independently selected from 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, the invention provides modified oligonucleotides which comprise oligonucleotides consisting of 5 to 6, 5 to 7, 5 to 8, 5 to 9, 5 to 5 to 10, 5 to 11, 5 to 12, 5 to 13, 5 to 14, 5 to 15, 5 to 16, 5 to 17, 5 to 18, 5 to 19, 5 to 20, 6 to 7 6 to 8, 6 to 9, 6 to 10, 6 to 11, 6 to 12, 6 to 13, 6 to 14, 6 to 15, 6 to 16, 6 to 17, 6 to 18, 6 to 19, 6 to 20, 7 to 8, 7 to 9, 7 to 10, 7 to 11, 7 to 12, 7 to 13, 7 to 14, 7 to 15, 7 to 16, 7 to 17, 7 to 18, 7 to 19, 7 to 20, 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligomeric compound or oligonucleotide is limited, whether to a range or to a specific number, the oligomeric compound or oligonucleotide may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugates, terminal groups, or other substituents. In certain embodiments, a modified oligonucleotide has any of the above lengths.

Further, where an oligonucleotide is described by an overall length range and by regions having specified lengths, and where the sum of specified lengths of the regions is less than the upper limit of the overall length range, the oligonucleotide may have additional nucleosides, beyond those of the specified regions, provided that the total number of nucleosides does not exceed the upper limit of the overall length range.

In certain embodiments, oligonucleotides of the present application are characterized by their modification motif and overall length. In certain embodiments, such parameters are each independent of one another.

### Oligomeric Compounds

In certain embodiments, the invention provides oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

In certain embodiments, antisense oligonucleotides are provided wherein the 5′-terminal group comprises a 5′-terminal stabilized phosphate. A “5′-terminal stabilized phosphate” is a 5′-terminal phosphate group having one or more modifications that increase nuclease stability relative to a 5′-phosphate.

In certain embodiments, antisense oligonucleotides are provided wherein the 5′-terminal group has Formula IIe:

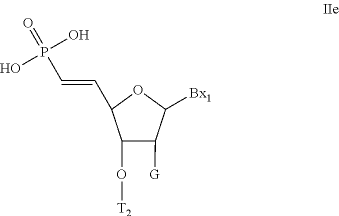

wherein:
Bx is uracil, thymine, cytosine, 5-methyl cytosine, adenine or guanine;
T2 is a phosphorothioate internucleoside linking group linking the compound of Formula lie to the oligomeric compound; and
G is halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O) N(H)CH₃, OCH₂C(═O)—N(H)—(CH₂)₂—N(CH₃)₂ or OCH₂—N(H)—C(═NH)NH₂.

In certain embodiments, antisense oligonucleotides are provided wherein said 5′-terminal compound has Formula IIe wherein G is F, OCH₃ or O(CH₂)₂—OCH₃.

In certain embodiments, the 5′-terminal group is a 5′-terminal stabilized phosphate comprising a vinyl phosphonate represented by Formula IIe above.

### Conjugate Groups

In certain embodiments, the ASO of the present application comprises an antisense oligonucleotide modified by covalent attachment of one or more conjugate groups (also referred to as “conjugate partner”). In general, conjugate groups modify one or more properties of the attached oligonucleotide including but not limited to pharmacodynamics, pharmacokinetics, stability, binding, absorption, cellular distribution, cellular uptake, charge and clearance. As used herein, “conjugate group” means a radical group comprising a group of atoms that are attached to an oligonucleotide or oligomeric compound. In general, conjugate groups modify one or more properties of the compound to which they are attached, including but not limited to, pharmacodynamics, pharmacokinetics, binding, absorption, cellular distribution, cellular uptake, charge and/or clearance properties. Conjugate groups are routinely used in the chemical arts and can include a conjugate linker that covalently links the conjugate group to an oligonucleotide or oligomeric compound. In certain embodiments, conjugate groups include a cleavable moiety that covalently links the conjugate group to an oligonucleotide or oligomeric compound. In certain embodiments, conjugate groups include a conjugate linker and a cleavable moiety to covalently link the conjugate group to an oligonucleotide or oligomeric compound. In certain embodiments, a conjugate group has the general formula:

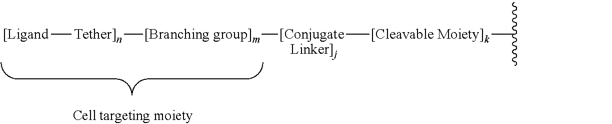

wherein n is from 1 to about 3, m is 0 when n is 1 or m is 1 when n is 2 or 3, j is 1 or 0, k is 1 or 0 and the sum of j and k is at least one.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

Conjugate groups are shown herein as radicals, providing a bond for forming covalent attachment to an oligomeric compound such as an oligonucleotide. In certain embodiments, the point of attachment on the oligomeric compound is at the 3′-terminal nucleoside or modified nucleoside. In certain embodiments, the point of attachment on the oligomeric compound is the 3′-oxygen atom of the 3′-hydroxyl group of the 3′ terminal nucleoside or modified nucleoside. In certain embodiments, the point of attachment on the oligomeric compound is at the 5′-terminal nucleoside or modified nucleoside. In certain embodiments the point of attachment on the oligomeric compound is the 5′-oxygen atom of the 5′-hydroxyl group of the 5′-terminal nucleoside or modified nucleoside. In certain embodiments, the point of attachment on the oligomeric compound is at any reactive site on a nucleoside, a modified nucleoside or an internucleoside linkage.

As used herein, “cleavable moiety” and “cleavable bond” mean a cleavable bond or group of atoms that is capable of being split or cleaved under certain physiological conditions. In certain embodiments, a cleavable moiety is a cleavable bond. In certain embodiments, a cleavable moiety comprises a cleavable bond. In certain embodiments, a cleavable moiety is a group of atoms. In certain embodiments, a cleavable moiety is selectively cleaved inside a cell or sub-cellular compartment, such as a lysosome. In certain embodiments, a cleavable moiety is selectively cleaved by endogenous enzymes, such as nucleases. In certain embodiments, a cleavable moiety comprises a group of atoms having one, two, three, four, or more than four cleavable bonds.

In certain embodiments, conjugate groups comprise a cleavable moiety. In certain such embodiments, the cleavable moiety covalently attaches the oligomeric compound to the conjugate linker. In certain such embodiments, the cleavable moiety covalently attaches the oligomeric compound to the cell-targeting moiety.

In certain embodiments, a cleavable bond is selected from among an amide, a polyamide, an ester, an ether, one or both esters of a phosphodiester, a phosphate ester, a carbamate, a di-sulfide, or a peptide. In certain embodiments, a cleavable bond is one of the esters of a phosphodiester. In certain embodiments, a cleavable bond is one or both esters of a phosphodiester. In certain embodiments, the cleavable moiety is a phosphodiester linkage between an oligomeric compound and the remainder of the conjugate group. In certain embodiments, the cleavable moiety comprises a phosphodiester linkage that is located between an oligomeric compound and the remainder of the conjugate group. In certain embodiments, the cleavable moiety comprises a phosphate or phosphodiester. In certain embodiments, the cleavable moiety is attached to the conjugate linker by either a phosphodiester or a phosphorothioate linkage. In certain embodiments, the cleavable moiety is attached to the conjugate linker by a phosphodiester linkage. In certain embodiments, the conjugate group does not include a cleavable moiety.

In certain embodiments, the cleavable moiety is a cleavable nucleoside or a modified nucleoside. In certain embodiments, the nucleoside or modified nucleoside comprises an optionally protected heterocyclic base selected from a purine, substituted purine, pyrimidine or substituted pyrimidine. In certain embodiments, the cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine.

In certain embodiments, the cleavable moiety is 2′-deoxy nucleoside that is attached to either the 3′ or 5′-terminal nucleoside of an oligomeric compound by a phosphodiester linkage and covalently attached to the remainder of the conjugate group by a phosphodiester or phosphorothioate linkage. In certain embodiments, the cleavable moiety is 2′-deoxy adenosine that is attached to either the 3′ or 5′-terminal nucleoside of an oligomeric compound by a phosphodiester linkage and covalently attached to the remainder of the conjugate group by a phosphodiester or phosphorothioate linkage. In certain embodiments, the cleavable moiety is 2′-deoxy adenosine that is attached to the 3′-oxygen atom of the 3′-hydroxyl group of the 3′-terminal nucleoside or modified nucleoside by a phosphodiester linkage. In certain embodiments, the cleavable moiety is 2′-deoxy adenosine that is attached to the 5′-oxygen atom of the 5′-hydroxyl group of the 5′-terminal nucleoside or modified nucleoside by a phosphodiester linkage. In certain embodiments, the cleavable moiety is attached to a 2′-position of a nucleoside or modified nucleoside of an oligomeric compound.

As used herein, “conjugate linker” in the context of a conjugate group means a portion of a conjugate group comprising any atom or group of atoms that covalently link the cell-targeting moiety to the oligomeric compound either directly or through the cleavable moiety. In certain embodiments, the conjugate linker comprises groups selected from alkyl, amino, oxo, amide, disulfide, polyethylene glycol, ether, thioether (—S—) and hydroxylamino (—O—N(H)—). In certain embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus linking group. In certain embodiments, the conjugate linker comprises at least one phosphodiester group. In certain embodiments, the conjugate linker includes at least one neutral linking group.

In certain embodiments, the conjugate linker is covalently attached to the oligomeric compound. In certain embodiments, the conjugate linker is covalently attached to the oligomeric compound and the branching group. In certain embodiments, the conjugate linker is covalently attached to the oligomeric compound and a tethered ligand. In certain embodiments, the conjugate linker is covalently attached to the cleavable moiety. In certain embodiments, the conjugate linker is covalently attached to the cleavable moiety and the branching group. In certain embodiments, the conjugate linker is covalently attached to the cleavable moiety and a tethered ligand. In certain embodiments, the conjugate linker includes one or more cleavable bonds. In certain embodiments, the conjugate group does not include a conjugate linker.

As used herein, “branching group” means a group of atoms having at least 3 positions that are capable of forming covalent linkages to two or more tether-ligands and the remainder of the conjugate group. In general, a branching group provides a plurality of reactive sites for connecting tethered ligands to the oligomeric compound through the conjugate linker and/or the cleavable moiety. In certain embodiments, the branching group comprises groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, the branching group is covalently attached to the conjugate linker. In certain embodiments, the branching group is covalently attached to the cleavable moiety. In certain embodiments, the branching group is covalently attached to the conjugate linker and each of the tethered ligands. In certain embodiments, the branching group comprises one or more cleavable bond. In certain embodiments, the conjugate group does not include a branching group.

In certain embodiments, conjugate groups as provided herein include a cell-targeting moiety that has at least one tethered ligand. In certain embodiments, the cell-targeting moiety comprises two tethered ligands covalently attached to a branching group. In certain embodiments, the cell-targeting moiety comprises three tethered ligands covalently attached to a branching group.

As used herein, “tether” means a group of atoms that connect a ligand to the remainder of the conjugate group. In certain embodiments, each tether is a linear aliphatic group comprising one or more groups selected from alkyl, substituted alkyl, ether, thioether, disulfide, amino, oxo, amide, phosphodiester and polyethylene glycol groups in any combination. In certain embodiments, each tether is a linear aliphatic group comprising one or more groups selected from alkyl, ether, thioether, disulfide, amino, oxo, amide and polyethylene glycol groups in any combination. In certain embodiments, each tether is a linear aliphatic group comprising one or more groups selected from alkyl, substituted alkyl, phosphodiester, ether and amino, oxo, amide groups in any combination. In certain embodiments, each tether is a linear aliphatic group comprising one or more groups selected from alkyl, ether and amino, oxo, amide groups in any combination. In certain embodiments, each tether is a linear aliphatic group comprising one or more groups selected from alkyl, amino and oxo groups in any combination. In certain embodiments, each tether is a linear aliphatic group comprising one or more groups selected from alkyl and oxo groups in any combination. In certain embodiments, each tether is a linear aliphatic group comprising one or more groups selected from alkyl and phosphodiester in any combination. In certain embodiments, each tether comprises at least one phosphorus linking group or neutral linking group.

In certain embodiments, tethers include one or more cleavable bond. In certain embodiments, each tethered ligand is attached to a branching group. In certain embodiments, each tethered ligand is attached to a branching group through an amide group. In certain embodiments, each tethered ligand is attached to a branching group through an ether group. In certain embodiments, each tethered ligand is attached to a branching group through a phosphorus linking group or neutral linking group. In certain embodiments, each tethered ligand is attached to a branching group through a phosphodiester group. In certain embodiments, each tether is attached to a ligand through either an amide or an ether group. In certain embodiments, each tether is attached to a ligand through an ether group.

In certain embodiments, each tether comprises from about 8 to about 20 atoms in chain length between the ligand and the branching group. In certain embodiments, each tether comprises from about 10 to about 18 atoms in chain length between the ligand and the branching group. In certain embodiments, each tether comprises about 13 atoms in chain length.

In certain embodiments, the present disclosure provides ligands wherein each ligand is covalently attached to the remainder of the conjugate group through a tether. In certain embodiments, each ligand is selected to have an affinity for at least one type of receptor on a target cell. In certain embodiments, ligands are selected that have an affinity for at least one type of receptor on the surface of a mammalian liver cell. In certain embodiments, ligands are selected that have an affinity for the hepatic asialoglycoprotein receptor (ASGP-R). In certain embodiments, each ligand is a carbohydrate. In certain embodiments, each ligand is, independently selected from galactose, N-acetyl galactoseamine, mannose, glucose, glucosamone and fucose. In certain embodiments, each ligand is N-acetyl galactoseamine (GalNAc). In certain embodiments, the targeting moiety comprises 1 to 3 ligands. In certain embodiments, the targeting moiety comprises 3 ligands. In certain embodiments, the targeting moiety comprises 2 ligands. In certain embodiments, the targeting moiety comprises 1 ligand. In certain embodiments, the targeting moiety comprises 3 N-acetyl galactoseamine ligands. In certain embodiments, the targeting moiety comprises 2 N-acetyl galactoseamine ligands. In certain embodiments, the targeting moiety comprises 1 N-acetyl galactoseamine ligand.

In certain embodiments, each ligand is a carbohydrate, carbohydrate derivative, modified carbohydrate, multivalent carbohydrate cluster, polysaccharide, modified polysaccharide, or polysaccharide derivative. In certain embodiments, each ligand is an amino sugar or a thio sugar. For example, amino sugars may be selected from any number of compounds known in the art, for example glucosamine, sialic acid, α-D-galactosamine, N-Acetylgalactosamine, 2-acetamido-2-deoxy-D-galactopyranose (GalNAc), 2-Amino-3-O-[®-1-carboxyethyl]-2-deoxy-β-D-glucopyranose (β-muramic acid), 2-Deoxy-2-methylamino-L-glucopyranose, 4,6-Dideoxy-4-formamido-2,3-di-O-methyl-D-mannopyranose, 2-Deoxy-2-sulfoamino-D-glucopyranose and N-sulfo-D-glucosamine, and N-Glycoloyl-α-neuraminic acid. For example, thio sugars may be selected from the group consisting of 5-Thio-β-D-glucopyranose, Methyl 2,3,4-tri-O-acetyl-1-thio-6-O-trityl-α-D-glucopyranoside, 4-Thio-β-D-galactopyranose, and ethyl 3,4,6,7-tetra-O-acetyl-2-deoxy-1,5-dithio-α-D-gluco-heptopyranoside.

In certain embodiments, conjugate groups as provided herein comprise a carbohydrate cluster. As used herein, “carbohydrate cluster” means a portion of a conjugate group wherein two or more carbohydrate residues are attached to a branching group through tether groups. (see, e.g., Maier et al., “Synthesis of Antisense Oligonucleotides Conjugated to a Multivalent Carbohydrate Cluster for Cellular Targeting,” Bioconjugate Chemistry, 2003, (14): 18-29, which is incorporated herein by reference in its entirety, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, (47): 5798-5808, for examples of carbohydrate conjugate clusters).

As used herein, “modified carbohydrate” means any carbohydrate having one or more chemical modifications relative to naturally occurring carbohydrates.

As used herein, “carbohydrate derivative” means any compound which may be synthesized using a carbohydrate as a starting material or intermediate.

As used herein, “carbohydrate” means a naturally occurring carbohydrate, a modified carbohydrate, or a carbohydrate derivative.

In certain embodiments, conjugate groups are provided wherein the cell-targeting moiety has the formula:

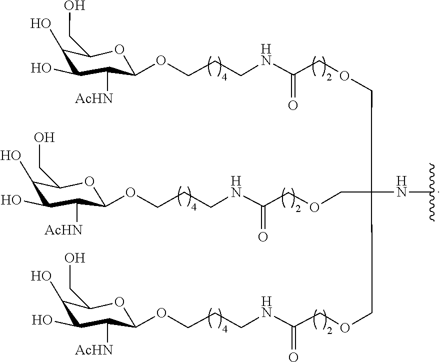

In certain embodiments, conjugate groups are provided wherein the cell-targeting moiety has the formula:

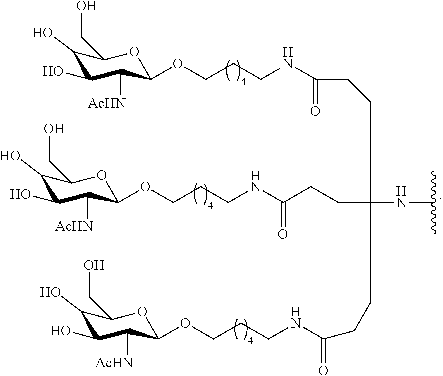

In certain embodiments, conjugate groups are provided wherein the cell-targeting moiety has the formula:

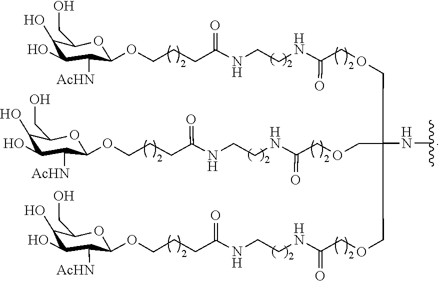

In certain embodiments, conjugate groups have the formula:

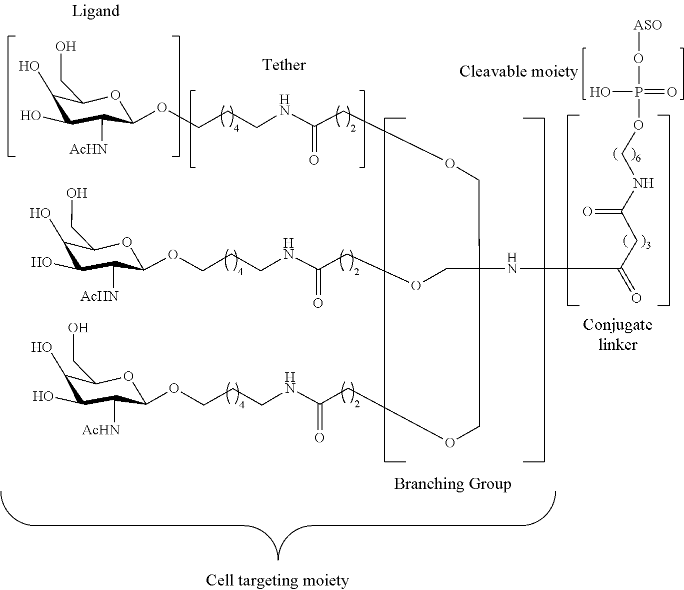

Representative United States patents, United States patent application publications, and international patent application publications that teach the preparation of certain of the above noted conjugate groups, conjugated oligomeric compounds such as ASOs comprising a conjugate group, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, each of which is incorporated by reference herein in its entirety.

Representative publications that teach the preparation of certain of the above noted conjugate groups, conjugated oligomeric compounds such as ASOs comprising a conjugate group, tethers, conjugate linkers, branching groups, ligands, cleavable moieties as well as other modifications include without limitation, BIESSEN et al., “The Cholesterol Derivative of a Triantennary Galactoside with High Affinity for the Hepatic Asialoglycoprotein Receptor: a Potent Cholesterol Lowering Agent” J. Med. Chem. (1995) 38:1846-1852, BIESSEN et al., “Synthesis of Cluster Galactosides with High Affinity for the Hepatic Asialoglycoprotein Receptor” J. Med. Chem. (1995) 38:1538-1546, LEE et al., “New and more efficient multivalent 50daman-ligands for asialoglycoprotein receptor of mammalian hepatocytes” Bioorganic & Medicinal Chemistry (2011) 19:2494-2500, RENSEN et al., “Determination of the Upper Size Limit for Uptake and Processing of Ligands by the Asialoglycoprotein Receptor on Hepatocytes in Vitro and in Vivo” J. Biol. Chem. (2001) 276(40):37577-37584, RENSEN et al., “Design and Synthesis of Novel N-Acetylgalactosamine-Terminated Glycolipids for Targeting of Lipoproteins to the Hepatic Asialoglycoprotein Receptor” J. Med. Chem. (2004) 47:5798-5808, SLIEDREGT et al., “Design and Synthesis of Novel Amphiphilic Dendritic Galactosides for Selective Targeting of Liposomes to the Hepatic Asialoglycoprotein Receptor” J. Med. Chem. (1999) 42:609-618, and Valentijn et al., “Solid-phase synthesis of lysine-based cluster galactosides with high affinity for the Asialoglycoprotein Receptor” Tetrahedron, 1997, 53(2), 759-770, each of which is incorporated by reference herein in its entirety.

In certain embodiments, conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantine, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or 4amantine acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

Some nonlimiting examples of conjugate linkers include pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include, but are not limited to, substituted C1-C10 alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

In certain embodiments, conjugate groups are at the 3′-end of an oligonucleotide of an oligomeric compound. In certain embodiments, conjugate groups are near the 3′-end. In certain embodiments, conjugates are attached at the 3′ end of an oligomeric compound, but before one or more terminal group nucleosides. In certain embodiments, conjugate groups are placed within a terminal group.

### III. Pharmaceutical Compositions

Another aspect of the present application relates to a pharmaceutical composition comprising one or more ASOs of the present application and a pharmaceutically acceptable carrier.

In some embodiments, the pharmaceutical composition comprises one or more anti-fibrosis gene-enhancing ASOs.

In some embodiments, the pharmaceutical composition comprises one or more pro-fibrosis gene-inhibiting ASOs. In some embodiments, the one or more pro-fibrosis gene-inhibiting ASOs are selected from the group consisting of SEQ ID NOS: 17 and 41.

In some embodiments, the pharmaceutical composition comprises (1) anti-fibrosis gene-enhancing ASOs and (2) one or more pro-fibrosis gene-inhibiting ASOs.

In some embodiments, the one or more anti-fibrosis gene-enhancing ASOs are selected from the group consisting of SEQ ID NOS:8, 9, 19, 15, 21, 45 and 46.

In some embodiments, the one or more pro-fibrosis gene-inhibiting ASOs are selected from the group consisting of SEQ ID NOS:17 and 41.

In some embodiments, the pharmaceutical composition comprises one or more carriers suitable for delivering the therapeutic agents to heart tissues. Exemplary carriers for delivery include nanoparticles, lipids, liposomes, micelles, polymers, polymeric micelles, emulsions, polyelectrolyte complexes, hydrogels, microcapsules, viruses, virus-like particle (VLPs), peptides, antibodies, aptamers, small molecule chemicals, exosomes, combinations thereof, and pegylated derivatives thereof. In a particular embodiment, the pharmaceutical composition comprises a nanoparticle formulation comprising an ASO in accordance with the present application.

In certain particular embodiments, the above-described carriers, including nanoparticles, may be linked to the heart tissue-specific targeting peptides or antibodies to facilitate carrier-mediated delivery of the active agents described herein to heart tissues. For example, in certain embodiments, pharmaceutical compositions include nanoparticles or liposomes covalently or non-covalently coated with a heart tissue-specific targeting peptide or antibody.

Exemplary nanoparticles include paramagnetic nanoparticles, superparamagnetic nanoparticles, metal nanoparticles, polymeric nanoparticles, nanoworms, nanoemulsions, nanogels, fullerene-like materials, inorganic nanotubes, dendrimers (such as with covalently attached metal chelates), nanocapsules, nanospheres, nanofibers, nanohorns, nano-onions, nanorods, nanoropes and quantum dots. A nanoparticle can produce a detectable signal, for example, through absorption and/or emission of photons (including radio frequency and visible photons) and plasmon resonance. Nanoparticles can be biodegradable or non-biodegradable.

In certain embodiments, the nanoparticle is a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199.

In other embodiments, a polymeric nanoparticle is made from a synthetic biodegradable polymer, a natural biodegradable polymer or a combination thereof. Synthetic biodegradable polymers can include, polyesters, such as poly(lactic-co-glycolic acid)(PLGA) and polycaprolactone; polyorthoesters, polyanhydrides, polydioxanones, poly-alkyl-cyano-acrylates (PAC), polyoxalates, polyiminocarbonates, polyurethanes, polyphosphazenes, or a combination thereof. Natural biodegradable polymers can include starch, hyaluronic acid, heparin, gelatin, albumin, chitosan, dextran, or a combination thereof.

In some embodiments, the pharmaceutical composition comprises a delivery carrier, such as a nanoparticle or liposome encapsulating a pharmaceutically effective amount of the antisense oligonucleotide. In some embodiments, the pharmaceutically effective amount of an ASO is from about 0.001 μg/mL to about 10 g/mL (w/v) of the pharmaceutically acceptable carrier. In some embodiments, the pharmaceutically effective amount of ASO is from about 0.1 μg/mL to about 1 μg/mL (w/v) of the pharmaceutically acceptable carrier.

In some embodiments, the pharmaceutical composition comprises an ASO of the present application and a lipid moiety. Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, the lipid moiety is selected to increase distribution of a pharmaceutical agent to heart tissue. In certain embodiments, the lipid moiety is selected to increase distribution of the pharmaceutical agent to heart muscle.

In certain embodiments, pharmaceutical compositions provided herein include one or more ASOs and one or more excipients. Exemplary excipients include water, salt solutions, alcohol, polyethylene glycols, gelatin, lactose, amylase, magnesium stearate, talc, silicic acid, viscous paraffin, hydroxymethylcellulose, polyvinylpyrrolidone and combinations thereof. In certain embodiments, the pharmaceutical compositions including one or more hydrophobic compounds, including organic solvents, such as dimethylsulfoxide.

In certain embodiments, the pharmaceutical composition provided herein comprises a co-solvent system. Co-solvent systems may include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied. For example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In some embodiments, the pharmaceutical composition comprises a sterile saline solution and one or more ASOs. In certain embodiments, the pharmaceutical composition consists of a sterile saline solution and one or more ASOs. In certain embodiments, the sterile saline is pharmaceutical grade saline. In certain embodiments, the pharmaceutical composition comprises one or more ASOs and sterile water. In certain embodiments, a pharmaceutical composition consists of one or more ASOs and sterile water. In certain embodiments, the sterile saline is pharmaceutical grade water. In certain embodiments, the pharmaceutical composition comprises one or more ASOs and phosphate-buffered saline (PBS). In certain embodiments, a pharmaceutical composition consists of one or more ASOs and sterile phosphate-buffered saline (PBS). In certain embodiments, the sterile saline is pharmaceutical grade PBS.

In certain embodiments, ASOs are admixed with pharmaceutically acceptable active and/or inert substances for the preparation of pharmaceutical compositions or formulations. Compositions and methods for the formulation of pharmaceutical compositions can depend on a number of criteria, including, but not limited to, route of administration, extent of disease, and/or dose to be administered.

Pharmaceutical compositions comprising ASOs may include any pharmaceutically acceptable salts, esters, or salts of such esters. In certain embodiments, pharmaceutical compositions comprising ASOs comprise one or more oligonucleotides, which, upon administration to an animal, such as a human, is capable of providing (directly or indirectly) the biologically active metabolite or residue thereof. Accordingly, for example, the disclosure is also drawn to pharmaceutically acceptable salts of ASOs, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an oligomeric compound which are cleaved by endogenous nucleases within the body, to form the active compound.

The pharmaceutical composition of the present application is formulated in accordance with the particular route of administration. Routes of administration for the therapeutic agents of the present application include oral and parenteral administration, i.e., injection, infusion, or implantation or by some other route other than the alimentary canal. Specific modes of administration include injections, such as intravenous, intramyocardial, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

In certain preferred embodiments, the pharmaceutical composition is formulated for administration by intravenous or intramyocardial injection. In certain embodiments, the pharmaceutical composition is formulated in aqueous solution, such as water or physiologically compatible buffers such as Hanks's solution, Ringer's solution, or physiological saline buffer. In certain embodiments, other ingredients are included (e.g., ingredients that aid in solubility or that serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Some pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

### IV. Methods for Treating Cardiac Fibrosis

Another aspect of the present application relates to a method for treating cardiac fibrosis in a subject. The method includes the step of administering to a subject in need of such treatment an effective amount of a pharmaceutical composition comprising an ASO of the present application.

In some embodiment, the method comprises the administration of a pharmaceutical composition comprising one or more anti-fibrosis gene-enhancing ASOs.

In some embodiment, the method comprises the administration of a pharmaceutical composition comprising one or more pro-fibrosis gene inhibiting ASOs.

In some embodiments, the method comprises the administration of a pharmaceutical composition comprises (1) anti-fibrosis gene enhancing ASOs and (2) one or more pro-fibrosis gene-inhibiting ASOs.

In some embodiments, the one or more anti-fibrosis gene-enhancing are selected from the group consisting of SEQ ID NOS:8, 9, 19, 15, 21, 45 and 46.

In some embodiments, the one or more pro-fibrosis gene-inhibiting ASOs are selected from the group consisting of SEQ ID NOS:17 and 41.

In another embodiment, the method comprises administration of a pharmaceutical composition containing an ASO formulated in a nanoparticle formulation.

In one embodiment, the method comprises administering the pharmaceutical composition to the subject intravenously or intramyocardially.

In some embodiments, the ASO dosage may be expressed as the amount of the compound that results in amelioration of symptoms or a prolongation of survival in a patient. Toxicity and therapeutic efficacy of the ASO can be determined by standard pharmaceutical procedures in cell cultures or experimental animals (e.g., for determining the LD50—the dose lethal to 50% of the population) and the ED50 (the dose therapeutically effective in 50% of the population). The dose ratio between toxic and therapeutic effects is the therapeutic index, which can be expressed as the ratio between LD50 and ED50. Compounds exhibiting high therapeutic indices are preferred. The data obtained from cell culture assays and animal studies can be used in formulating a range of dosages or amounts for use in mammals (e.g., humans). The dosage or amount of an ASO preferably lies within a range of circulating concentrations that include the ED50 with little or no toxicity. The dosage or amount may vary within this range depending upon the dosage form employed and the route of administration utilized. The exact formulation, route of administration and dosage can be chosen by the individual physician in view of the patient's condition. Dosage amount and interval may be adjusted individually to provide plasma levels of the active moiety which are sufficient to maintain the desired effects.

In certain embodiments, the ASO can be administered to a mammal having, suspected of having, or at risk of cardiac fibrosis and/or related pathologies at an amount sufficient to reduce target protein expression or activity. In accordance with certain embodiments, the ASO can be administered in a dose suitable for reducing target protein expression by at least 1%, 5%, 10%, 15%, 20%, 25%, 30%, 35%, 40%, 45%, 50%, 55%, 60%, 65%, 70%, 75%, 80%, 85%, 90%, 95%, or any range thereof.

Dosages for a particular patient can be determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose can be determined by the efficacy of the particular formulation, and the activity, stability or serum half-life of the ASO employed and the condition of the patient, as well as the body weight or surface area of the patient to be treated. The size of the dose can also be determined by the existence, nature, and extent of any adverse side-effects that accompany the administration of a particular composition in a particular patient.

Optimal precision in achieving effective ASO concentrations within a range yielding maximum efficacy with minimal toxicity may require a regimen based on the kinetics of the pharmaceutical composition's availability to the targeted heart tissues. Distribution, equilibrium, and elimination of a pharmaceutical composition may be considered when determining the optimal concentration for a treatment regimen. Generally, the pharmaceutical compositions of the present invention may be administered in a manner that maximizes efficacy and minimizes toxicity.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See e.g., U.S. Pat. No. 6,747,002, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 g, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The pharmaceutical compositions may be administered on a regimen of about 1 to about 10 times per day.

In the case of injections, it is usually convenient to give by an intravenous route in an amount of about 0.01 g-30 mg, about 0.01 μg-20 mg or about 0.01-10 mg per day to adults (at about 60 kg). In the case of other animals, the dose calculated for 60 kg may be administered as well.

Doses of a pharmaceutical composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 m/ml serum concentration per single or multiple administration or any range, value or fraction thereof.

As a non-limiting example, treatment of humans or animals can be provided as a onetime or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days. The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

### Combination Therapy

In some embodiments, the ASO of the present application can be administered in combination with one or more other therapeutic agents. The ASO of the present application and other therapeutic agents can be administered simultaneously or sequentially by the same or different routes of administration. The determination of the identity and amount of therapeutic agent(s) for use in the methods described herein can be readily made by ordinarily skilled medical practitioners using standard techniques known in the art. Generally, the ASO of the present application is administered in combination with an effective amount of another therapeutic agent that treats cardiac fibrosis and/or any heart disease or heart disease symptom associated with cardiac fibrosis.

Other therapeutic agents include, but are not limited to, beta blockers, anti-hypertensives, cardiotonics, anti-thrombotics, vasodilators, hormone antagonists, inotropes, diuretics, endothelin antagonists, calcium channel blockers, phosphodiesterase inhibitors, ACE inhibitors, angiotensin type 2 antagonists and cytokine blockers/inhibitors, and HDAC inhibitors.

More specifically, an ASO may be combined with another therapeutic agent including, but not limited to, an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In specific embodiments, the ASO of the present application is combined with an antihyperlipoproteinemic agent including aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof, acifran, azacosterol, benfluorex, β-benzalbutyramide, carnitine, chondroitin sulfate, clomestrone, detaxtran, dextran sulfate sodium, eritadenine, furazabol, meglutol, melinamide, mytatrienediol, ornithine, γ-oryzanol, pantethine, pentaerythritol tetraacetate, phenylbutyramide, pirozadil, probucol (lorelco), β-sitosterol, sultosilic acid-piperazine salt, tiadenol, triparanol and xenbucin.

In some embodiments, the ASO of the present application is combined with an antiarteriosclerotic agent such as pyridinol carbamate. In other embodiments, the ASO is combined with an antithrombotic/fibrinolytic agent including, but not limited to anticoagulants (acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin); anticoagulant antagonists, antiplatelet agents (aspirin, a dextran, dipyridamole (persantin), heparin, sulfinpyranone (anturane) and ticlopidine (ticlid)); thrombolytic agents (tissue plaminogen activator (activase), plasmin, pro-urokinase, urokinase (abbokinase) streptokinase (streptase), anistreplase/APSAC (eminase)); thrombolytic agent antagonists or combinations thereof).

In other embodiments, the ASO is combined with a blood coagulant including, but not limited to, thrombolytic agent antagonists (amiocaproic acid (amicar) and tranexamic acid (amstat); antithrombotics (anagrelide, argatroban, cilstazol, daltroban, defibrotide, enoxaparin, fraxiparine, indobufen, lamoparan, ozagrel, picotamide, plafibride, tedelparin, ticlopidine and triflusal); and anticoagulant antagonists (protamine and vitamin KI).

Alternatively, the ASO may be combined with an antiarrhythmic agent including, but not limited to, Class I antiarrythmic agents (sodium channel blockers), Class II antiarrythmic agents (beta-adrenergic blockers), Class III antiarrythmic agents (repolarization prolonging drugs), Class IV antiarrhythmic agents (calcium channel blockers) and miscellaneous antiarrythmic agents. Non-limiting examples of sodium channel blockers include Class IA (disppyramide (norpace), procainamide (pronestyl) and quinidine (quinidex)); Class IB (lidocaine (xylocalne), tocamide (tonocard) and mexiletine (mexitil)); and Class IC antiarrhythmic agents, (encamide (enkaid) and fiecamide (tambocor)).

Non-limiting examples of a beta blocker (also known as a β-adrenergic blocker, a β-adrenergic antagonist or a Class II antiarrhythmic agent) include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfmalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol. Non-limiting examples of an agent that prolongs repolarization, also known as a Class III antiarrhythmic agent, include amiodarone (cordarone) and sotalol (betapace).

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexyline. In certain embodiments a calcium channel blocker comprises a long-acting dihydropyridine (nifedipine-type) calcium antagonist.

Non-limiting examples of miscellaneous antiarrhymic agents include adenosine (adenocard), digoxin (lanoxin), acecamide, ajmaline, amoproxan, aprindine, bretylium tosylate, bunaftine, butobendine, capobenic acid, cifenline, disopyranide, hydroquinidine, indecamide, ipatropium bromide, lidocaine, lorajmine, lorcamide, meobentine, moricizine, pirmenol, prajmaline, propafenone, pyrinoline, quinidine polygalacturonate, quinidine sulfate and viquidil.

In other embodiments, the ASO of the present application ASO is combined with an antihypertensive agent including, but not limited to, alpha/beta blockers (labetalol (normodyne, trandate)), alpha blockers, anti-angiotensin II agents, sympatholytics, beta blockers, calcium channel blockers, vasodilators and miscellaneous antihypertensives.

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

Non-limiting examples of anti-angiotension II agents include angiotensin converting enzyme inhibitors and angiotension II receptor antagonists. Non-limiting examples of angiotensin converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan. Non-limiting examples of a sympatholytic include a centrally acting sympatholytic or a peripherally acting sympatholytic. Non-limiting examples of a centrally acting sympatholytic, also known as a central nervous system (CNS) sympatholytic, include clonidine (catapres), guanabenz (wytensin), guanfacine (tenex) and methyldopa (aldomet). Non-limiting examples of a peripherally acting sympatholytic include a ganglion blocking agent, an adrenergic neuron blocking agent, a β-adrenergic blocking agent or an al-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alphal-adrenergic blocker include prazosin (minipress), doxazocin (cardura) and terazosin (hytrin).

In certain embodiments, an antihypertensive agent may comprise a vasodilator (e.g., a cerebral vasodilator, a coronary vasodilator or a peripheral vasodilator). In particular embodiments, a vasodilator comprises a coronary vasodilator including, but not limited to, amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(P-diethylaminoethyl ether), hexobendine, itramin tosylate, khellin, lidoflanine, mannitol hexanitrane, medibazine, nicorglycerin, pentaerythritol tetranitrate, pentrinitrol, perhexyline, pimethylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil. In certain aspects, an antihypertensive may comprise an arylethanolamine derivative (amosulalol, bufuralol, dilevalol, labetalol, pronethalol, sotalol and sulfmalol); a benzothiadiazine derivative (althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlonnethiazide); a N-carboxyalkyl(peptide/lactam) derivative (alacepril, captopril, cilazapril, delapril, enalapril, enalaprilat, fosinopril, lisinopril, moveltipril, perindopril, quinapril and ramipril); a dihydropyridine derivative (amlodipine, felodipine, isradipine, nicardipine, nifedipine, nilvadipine, nisoldipine and nitrendipine); a guanidine derivative (bethanidine, debrisoquin, guanabenz, guanacline, guanadrel, guanazodine, guanethidine, guanfacine, guanochlor, guanoxabenz and guanoxan); a hydrazines/phthalazine (budralazine, cadralazine, dihydralazine, endralazine, hydracarbazine, hydralazine, pheniprazine, pildralazine and todralazine); an imidazole derivative (clonidine, lofexidine, phentolamine, tiamenidine and tolonidine); a quanternary ammonium compound (azamethonium bromide, chlorisondamine chloride, hexamethonium, pentacynium bis(methylsulfate), pentamethonium bromide, pentolinium tartrate, phenactropinium chloride and trimethidinium methosulfate); a reserpine derivative (bietaserpine, deserpidine, rescinnamine, reserpine and syrosingopine); or a sulfonamide derivative (ambuside, clopamide, faro semide, indapamide, quinethazone, tripamide and xipamide).

In other embodiments, the ASO of the present application is combined with a vasopressor. Vasopressors generally are used to increase blood pressure during shock, which may occur during a surgical procedure. Non-limiting examples of a vasopressor, also known as an antihypotensive include amezinium methyl sulfate, angiotensin amide, dimetofrine, dopamine, etifelmin, etilefrin, gepefrine, metaraminol, midodrine, norepinephrine, pholedrine and synephrine.

In some embodiments, the ASO of the present application is combined with treatment agents for congestive heart failure including, but not limited to, anti-angiotension II agents, afterload-preload reduction treatment (hydralazine (apresoline) and isosorbide dinitrate (isordil, sorbitrate)), diuretics, and inotropic agents.

Non-limiting examples of a diuretic include a thiazide or benzothiadiazine derivative (e.g., althiazide, bendroflumethazide, beizthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticrnafen and urea.

Non-limiting examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a 3-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for hypertrophy, vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

The present application is further illustrated by the following examples that should not be construed as limiting. The contents of all references, patents, and published patent applications cited throughout this application, as well as the Figures and Tables, are incorporated herein by reference.

### EXAMPLES

### Example 1: Materials and Methods

### Standardized Procedures

### ASO Handling

Each ASO (IDT) was reconstituted in a tube with RNAse free water.

An aliquot of 20-50 ul per tube was prepared to prevent freeze-thaw cycles.

### Cell Seeding and Transfection Condition

Cells were seeded in a 12-well plate and a 6-well plate for total RNA and protein isolation, respectively.

Cells were seeded to achieve 40-50% confluency before transfection.

HEK293T cells were transfected using Lipofectamine 3000 (0.2% concentration), while IHCFs were transfected using LipoRNAiMAX (0.35% concentration).

A transfection of 50 nM of ASOs was performed for 24 hours.

### Western Blotting Loading

A gradient curve was created on the left side of the gel by loading control samples at concentrations of 1×, 0.5× and 0.25×.

Following the gradient curve, the experimental samples were loaded.

### Quantification

The intensity of the gradient curve band was quantified using ImageI.

A standard curve was plotted, and an equation was derived.

The intensity of the experimental sample bands was quantified using ImageI.

An arbitrary value was obtained using the equation from the standard curve.

The sample values were normalized with the loading controls (alpha-tubulin, beta-actin or GAPDH).

### Detailed Procedures

### Cell Culture and Transfections

Human HEK293T cells were propagated in Dulbecco's modified Eagle's medium (DMEM; Gibco), and AC16 were propagated in an equal mix of F12:DMEM media (Gibco). Both media were supplemented with 10% fetal bovine serum. When ASO testing, 5×10⁵ cells HEK293T or AC16 cells were seeded in 10 cm dishes. Once adhered overnight, the culture medium was changed to OPTI-MEM (Gibco) and transfected with 50 nM ASOs using RNAiMAX (Thermo Fisher Scientific) following manufacture's guidelines for 6 hr, after which the medium is changed back to the regular culture medium. Cells were harvested 18 hr after transfection.

### Western Blotting

Cells were lysed in RIPA buffer (Thermo Fisher Scientific), and total cell proteins were separated in a 6%-15% denaturing polyacrylamide gel, transferred to polyvinylidene difluoride membranes (PVDF; Amersham Biosciences), and probed using antibodies recognizing GATA4 (Santa Cruz), R-actin (Thermo Fisher Scientific), DDX3X (Sigma), then incubated with either a mouse or rabbit secondary antibody conjugated with horseradish peroxidase (GE Biosciences). Blots were quantified using ImageI (NIH).

### Dual-Luciferase Assay

Untreated HEK293T cells were seeded in 96 well plates at a density of 1×10⁴ cells per well and left to adhere overnight. The cells are then transfected each with 50 ng 5′UTR-FLuc reporter plasmid and a control renilla luciferase (RLuc) plasmid using Lipofectamine 3000 (Thermo Fisher Scientific) based on manufacture's guidelines. After 24 hours, the cells were incubated with Dual-Glo luciferase substrated (Promega) according to the manufacturer's recommendations. The final readings of the Fluc are then normalized to Rluc to obtain the relative luminescence reading.

### RNA Purification and RT-qPCR

For ASO transfected cells the media was aspirated from adherent cells and then washed twice with chilled PBS. The cells are then lysed by adding 1000 ul of Trizol (Qiagen) directly to the cells, which is then mixed with 200 ul of chloroform in 1.5 ml tubes, and left to on ice for 5 min. The mixture is then spun down at 16,000 g for 10 min. RNA is then precipitated from the aqueous layer by adding two volumes of isopropanol and spinning down at 16,000 g for 10 min. The pellet is then washed twice with 70% ethanol, left to dry, and resuspended in nuclease-free water. For quantitation of mRNA levels, cDNAs were prepared using iScript master mix RT Kit (Biorad) and subsequently qPCR-amplified using SYBR Primer Assay kits (Biorad). Notably, when a primer set was first used, the identity of the resulting PCR product was confirmed by cloning and sequencing. The quantitative nature of each primer was also assessed by performing a standard curve of varying cDNA amount. Once confirmed, melting curves were used in each subsequent PCR to verify that each primer set reproducibly and specifically generates the same PCR product.

### Polysome Profiling and RNA Extraction

Following ASO transfection, cells are incubated with cycloheximide (100 μg/ml; Sigma) for 10 min and then harvested using a native lysis buffer with 100 mM KCl, 5 mM MgCl2, 10 mM HEPES, pH 7.0, 0.5% Nonidet P-40, 1 mM DTT, 100 U ml-1 RNasin RNase inhibitor (Promega), 2 mM vanadyl ribonucleoside complexes solution (Sigma-Aldrich (Fluka BioChemika)), 25 μl ml-1 protease inhibitor cocktail for mammalian tissues (Sigma-Aldrich), cycloheximide (100 ug/ml). The lysate is then spun down at 1500 g for 5 min to pellet the nuclei. The supernatant is then loaded onto a 10-50% sucrose gradient and spun in an ultracentrifuge at 150,000 g for 2 hours and 20 min. The gradients are then transferred to a fractionator coupled to an ultraviolet absorbance detector that outputs an electronic trace across the gradient. Using a 60% sucrose chase solution, the gradient is then pumped into the fractionator, and divided equally into 12 fractions. RNA was extracted by mixing 500 ul of each fraction is mixed with equal volumes of chloroform:phenol:chloroform:isoamyl alcohol (25:24:1) and 0.1 volumes of 3M sodium acetate (PH 5.2), then spun down at 16,000 g for 10 min. The upper aqueous layer was then used to repeat this extraction process. The final upper aqueous later is then mixed with two volumes of 100% ethanol and left to incubate at −20 overnight. The solution is then spun at maximum speed for 30 min to pellet the RNA, which is then washed twice with 70% ethanol, and finally resuspended in nuclease-free water.

### Immunofluorescence

WGA (5 mg) was dissolved in 5 ml of PBS (pH 7.4). We performed deparaffinization by following steps: (i) Xylene (100%) for 2×5 mins; (ii) Ethanol (100%) for 2×5 mins; (iii). Ethanol (95%) for 1×5 mins; (iv) ddH2O for 2×5 min. The slides were kept in a pressure cooker for 10 min along with citrate buffer (10 mM, pH 6.0) for antigen retrieval. We quenched the slides with 0.1 M glycine in phosphate buffer (pH 7.4) for 1 h at RT. Circles were made with a Dako pen, and slides were blocked with goat normal serum for 30 min. 10 g/ml of WGA-Alexa Fluor 488 (Sigma Aldrich) was applied to the slides for incubation for 1 h at RT. Slides were rinsed in PBS 3×5 min. A coverslip was placed on the slides with VECTASHIELD HardSet antifade mounting medium with DAPI (Vectorlabs) for imaging. For AC16 and ESC derived CM cultures, the cells were fixed using a 4% paraformaldehyde in PBS for 10 min, washed with PBS, and permeabilized using 0.2% Triton X-100 for 10 min. Cells were blocked in 2% BSA/PBS for 1 h and stained with the appropriate primary antibody for 1 hour min at RT, then incubated with secondary fluorescently labelled antibodies. The stained cells were gently washed with PBS for 3×5 min, and the slides were mounted using a mounting medium with DAPI. When measuring cell size surface area in heart sections or AC16 slides, five different fields were selected, and the cell size of at least 200 CM cells was measured using Image J. For ESC-derived-CM, it was found it hard to measure cell surface area of these cells in isolation as with AC16 cells. Therefore, we used Image J to measure the whole surface whole colony surface area for at least five colonies per sample, which is then divided by the number of nuclei.

### Statistics

All quantitative data were presented as mean±SD and analyzed using Prism 8.3.0 software (GraphPad). For a comparison between two groups, an unpaired two-tailed Student t-test for normally distributed data were performed. For mouse echocardiography studies, ANOVA followed by Holm-Sidak post hoc test was used to determine the statistical significance among groups. Two-sided P values <0.05 were considered to indicate statistical significance. Specific statistical methods were described in the figure legends.

Sequences of ASOs used in this study (5′-3′; “m” indicates a 2′-O-methyl modification, “e” indicates a 2′-O-methoxyethyl modification, “+” indicates LNA, “s” indicates phosphorothioate, and “o” indicates a phosphodiester internucleoside linkage). GATA4 human ASO control: used in human and mouse as a control; underline: mismatch compared to GATA4 ASO1.

All the ASOs were synthesized in IDT, inc. 100 nmoles, purified using a desalting column. Analytical ESI-MS confirmed the purity and quality of the ASOs. All the ASOs were synthesized in IDT, inc. 100 nmoles, purified using a desalting column. Analytical ESI-MS confirmed the purity and quality of the ASOs.

FIG. 10 lists the following: the human GATA4 mRNA sequence (SEQ ID NO:22), mouse GATA4 mRNA sequence (SEQ ID NO:23), human MEF2C mRNA sequence (SEQ ID NO:24), human NKX2-5 mRNA sequence (SEQ ID NO:25), and human eIF4G2 mRNA sequence (SEQ ID NO:26), GATA4 human type I uotASO target sequence (SEQ ID NO:27), GATA4 human type II motASO target sequence (SEQ ID NO:28), MEF2C human type II motASO target sequence (SEQ ID NO:29), NKX2-5 human type II motASO target sequence (SEQ ID NO:30), eIF4G2 human type II uotASO target sequence (SEQ ID NO:31), human, gorilla and monkey GATA4 uORF DNA sequence (SEQ ID NO:32), cat and dolphin GATA4 uORF DNA sequence (SEQ ID NO:33), golden hamster GATA4 uORF DNA sequence (SEQ ID NO:34), rat GATA4 uORF DNA sequence (SEQ ID NO:35), mouse GATA4 uORF DNA sequence (SEQ ID NO:36), WT human GATA4 stem loop region (SEQ ID NO:37), ΔuORF human GATA4 stem loop region (SEQ ID NO:38), Mut human GATA4 stem loop region (SEQ ID NO:39), Rescue Mut human GATA4 stem loop region (SEQ ID NO: 40), EIF4G2-ASO-1-gapmer (SEQ ID NO: 41), Unmodified EIF4G2-ASO-1 (SEQ ID NO: 42), Human MYBPC3 mRNA (SEQ ID NO: 43), Human CRYAB mRNA (SEQ ID NO: 44), MYBPC3 ASO (SEQ ID NO: 44), and CRYAB ASO (SEQ ID NO: 46).

### Example 2: Downstream dsRNA Structure Adjacent to uORF Inhibits Translation of mORF

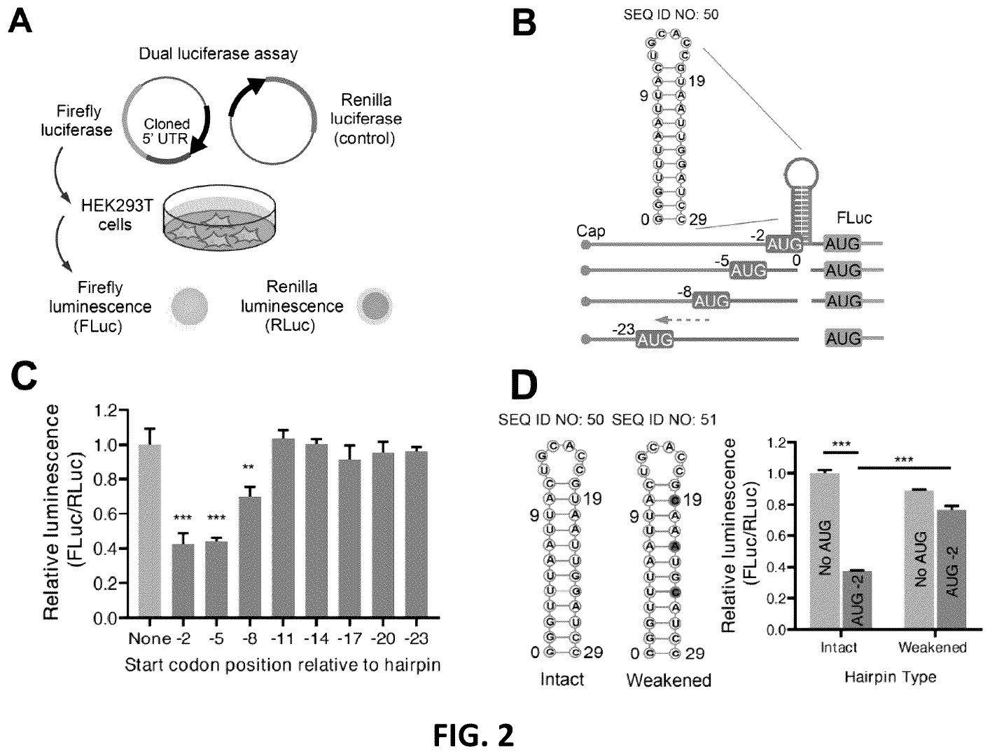

Double-stranded RNA (dsRNA) structures embedded in 5′UTRs have been reported to inhibit or activate translation dependent on its location and structure features. In addition, upstream open reading frames (uORFs) are known to inhibit the main open reading frame (mORF) translation. To explore the potential crosstalk between such dsRNA structures and uORFs, artificial luciferase reporters containing dsRNA and uORF elements for dual-luciferase reporter assays were constructed (FIG. 2, panel A).

Specifically, a series of 5′UTR-firefly luciferase (FLuc) reporter fusions were created from a 5′UTR containing a CA repeat backbone with a stable hairpin Kan-HP1 40-nt away from the 5′-end and 20-nt away from the FLuc ORF start codon. The 5′UTR was synthesized as oligonucleotides (for both + and − strands) from IDT and then cloned into a FLuc construct that corresponds to mORF. The 5′UTR backbone contained a CA repeat (i.e., [CA]*n) which is known to be a linear sequence. In this backbone, the hairpin was added 40-nt away from the 5′ end and 20-nt away from the firefly mORF coding sequence. The hairpin was obtained from the Disney paper because it contained a G at the beginning of it so if an AU is placed before it, it creates a uORF. The AUG was then shifted backward by 3 nucleotides for every reporter up to 27. This backbone was then mutagenized by inserting start codons (i.e., ATG) at various positions spaced by 2 nt up to 23 nt relative to the base of the stem (FIG. 2, panel B) Dual-Luc assays showed that start codons at positions −2 and −5 confer the most robust suppression of the luciferase activity; weaker inhibition was conferred at position −8 (FIG. 2, panel C). Start codons at positions −8 to −23 conferred no detectable suppression.

To elucidate the role of hairpin stability on uORF activity, mismatches were introduced into the hairpin of the parent construct (not containing AUG) as well as the one containing a start codon at position −2 (AUG −2) (FIG. 2, panel D, left). Mutations in the parent sequence confer no suppression over the luciferase activity, whereas mutations in the AUG −2 variant showed rescuing in the luciferase activity, suggesting that the RNA structure stability is needed for the AUG codon to confer suppression. Compared to the parent AUG −2 construct, the mutagenized AUG −2 variants exhibited enhanced luciferase activity and protein levels (FIG. 2, panel D, right). Enhanced luciferase activity was also observed from a parent construct in which the AUG was deleted. Taken together, these results demonstrate a functional connection between upstream ATGs or uORFs, RNA structure stability and translation initiation of mORFs. In particular, a dsRNA stem-loop RNA structure located at a proximal location downstream of uORF (2-11 nt away) was found to enhance uORF activity and reduce mORF translation. These results lend support to the discovery that dsRNA stem-loop structures immediately downstream of uORFs can enhance uORF activity and suppress translation of a mORF.

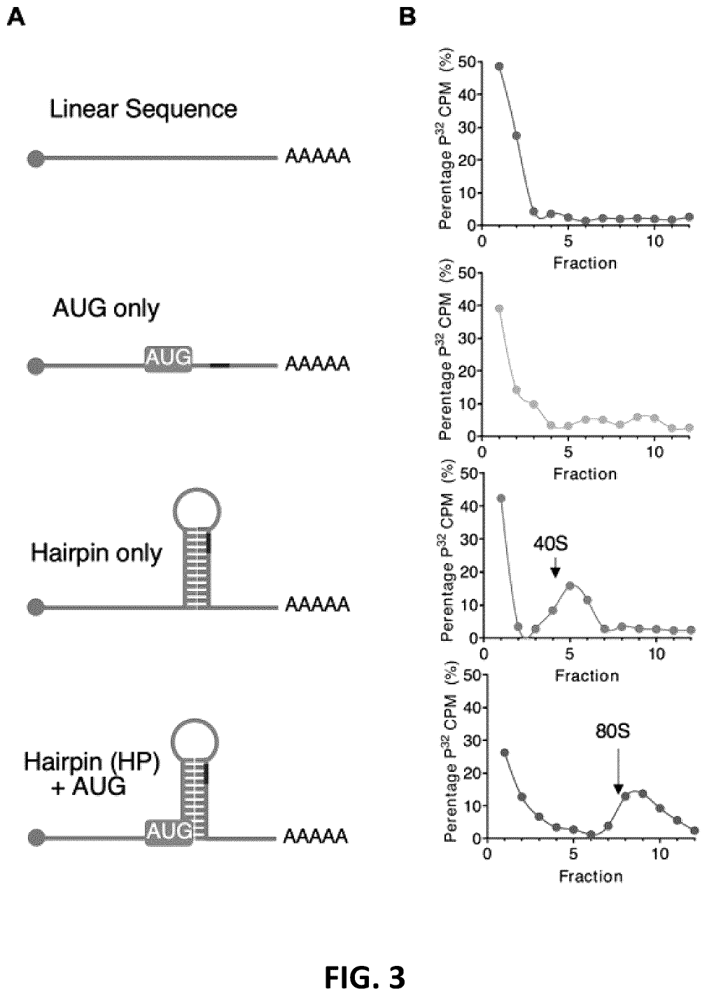

To explore the mechanism underlying the dsRNA-mediated shift of uORF to mORF translation, in vitro transcription of a series of mRNAs with 5′UTRs based on the constructs used in FIG. 2, Panel B, was done and then they were incubated in the rabbit reticulocyte lysate (RRL) (FIA). The lysate was then fractionated on a 10-35% sucrose gradient by ultracentrifugation. The hairpin-bearing 5′UTR of the non-AUG-containing reporter with RRL resulted in co-sedimentation of the 5′UTR with the 40S ribosomal subunit, which was not observed in a control 5′UTR lacking the hairpin and a start codon (FIG. 3, Panel B, in red), suggesting a hairpin-specific co-sedimentation effect (FIG. 3, Panel B, in cyan). Whereas, coupling a start codon with an adjacent downstream hairpin resulted in a shift from the 40S peak in the profile towards an assembled 80S monosome (FIG. 3, Panel B, in green) to a greater extent than a start codon alone (FIG. 3, Panel B, in yellow), suggesting enhanced translation initiation by a hairpin structure downstream of a start codon. Taken together, these results indicate that the presence of a hairpin downstream of a uORF initiation codon enhances the suppressive capability of the uORF against mORF translation, and this synergistic effect between the start codon and the hairpin dsRNA is abolished when the hairpin stem is destabilized.

### Example 3: Presence of uORFs in Human Cardiac Transcriptional Factor mRNAs

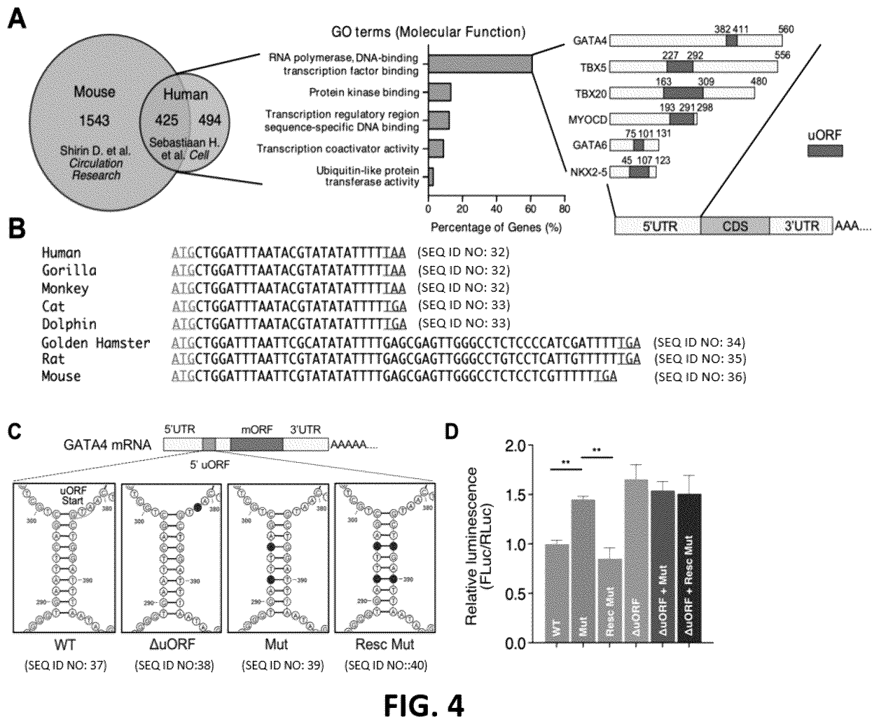

To further investigate a role for dsRNA stem-loop RNA structures and uORFs in suppressing translation of mORFs, a search was conducted to identify naturally existing mRNA transcripts containing one or more uORFs within or surrounding dsRNA structural elements. This was carried out by data mining of unbiased high throughput ribosome profiling (Ribo-seq) databases. Overlapping of Ribo-seq hits uncovered a conserved cohort of mRNAs containing translating uORFs in mice and humans (FIG. 4, panel A, left, middle). Gene ontology analysis of overlapping genes revealed transcription factors as the top enriched gene set containing translatable uORF, including GATA4, GATA6, TBX5, TBX20, MYOCD, and NKX2-5 (FIG. 4, panel A, right).

Among these 6 transcription factors, GATA4 was of particular interest since GATA4 mRNA contains a single uORF exhibiting ribosome footprints in the human heart by Ribo-seq analysis (FIG. 4, panel A, right) and since the GATA4 uORF is conserved across various mammals and includes an 11-nt sequence downstream of the uORF start codon that is highly conserved through evolution. However, the fact that the uORF protein sequences are not conserved suggests that the GATA4 uORF is more likely to be a regulatory element rather than a bioactive peptide.

GATA4 is a key transcription factor required for cardiomyocyte growth and hypertrophy. RNA structure prediction by the TurboFold tool suggested the presence of a 10 base-pair (bp) stem directly downstream of the uORF start codon shown in the illustration of the predicted structure of 5′UTR (FIG. 4, panel C). A Selective 2′ Hydroxyl Acylation analyzed by Primer Extension (SHAPE) assay was used to confirm the existence of the double stranded secondary stem structure in the 5′UTR of GATA4 mRNA. In brief, nucleotides located in double-stranded stem structures tend to be less modified by the electrophile, N-methylisatoic anhydride (NAI), while single-stranded regions are exposed for more intense modification. Confirmation of the double-stranded RNA structure was obtained by showing that the predicted 10-bp stem loop downstream of the AUG start codon exhibited the lowest SHAPE activity (data not shown). This result was further evidenced by experiments showing stalling of the 40S ribosome subunit at the hairpin dsRNA region using a toe-printing assay (data not shown).

### Example 4: GATA4-Targeting ASOs Regulate GATA4 mORF Translation Efficiency in Cells

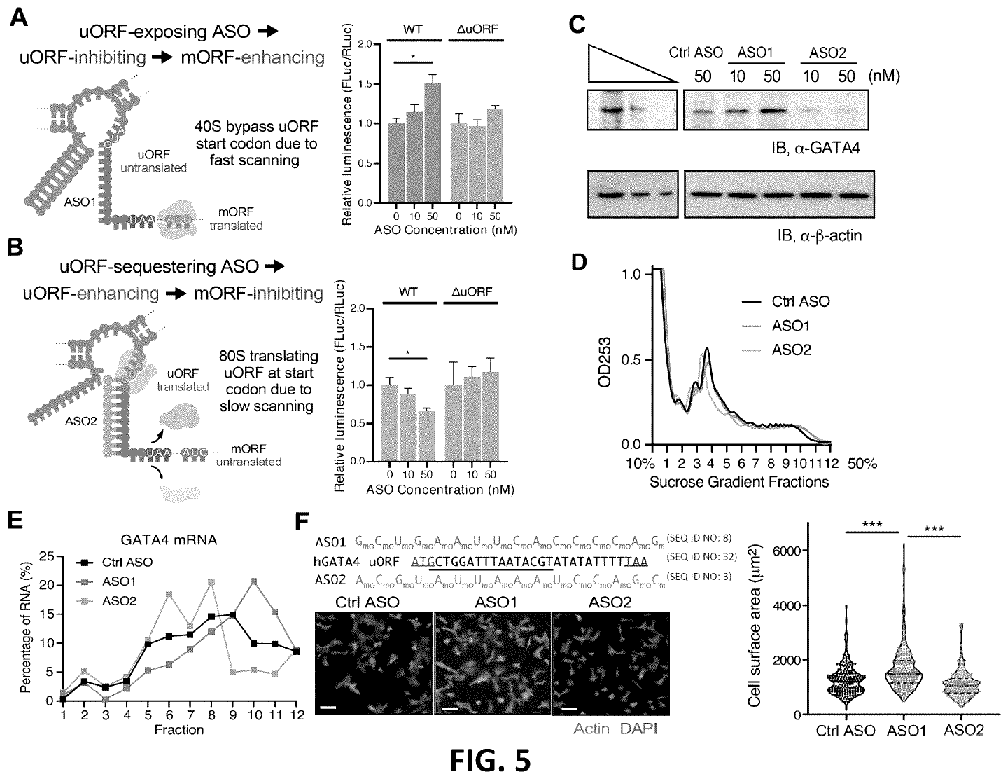

The GATA4 5′UTR variant studies provided an impetus for examining the potential therapeutic effects of using 5′UTR-directed agonists or antagonists to modify GATA4 expression in a therapeutic context. Such studies are predicated on perturbing the activities of the GATA 4 uORF and mORF relative to one another. In this regard, two hypotheses were considered: 1) Disruption of dsRNA structure leads to inactivation of uORF and higher Luc activity; and 2) Sequestration of the uORF results in an increase in its translation, resulting in less Luc activity. The first hypothesis was tested by designing a uORF-suppressing 16-mer ASO (human ASO1, SEQ ID NO:8) mimicking the disruption of the upstream strand by preventing it from the sequestering the uORF-containing strand (FIG. 5, panel A, left). The second hypothesis was tested by designing an uORF-enhancing ASO (human ASO2, SEQ ID NO:3) that can tightly sequester the uORF due to complementary binding, thereby forming a stable 16 bp double-stranded stem (FIG. 5, panel B, left).

In dual-Luc assays, ASO1 increased Luc activity, suggesting inhibition of uORF translation, while ASO2 decreased Luc activity, suggesting activation of uORF translation (FIG. 5, panel A, right). These effects are uORF dependent, as indicated by the fact that the ΔuORF reporter activity was unchanged with both ASOs (FIG. 5, panel B, right).

Targeting the endogenous GATA4 mRNA in AC16 human CMs with these ASOs led to observable protein level changes (FIG. 5, panel C). The uORF-suppressing ASO1 increased GATA4 protein levels, while the uORF-enhancing ASO2 reduced it. To further confirm the uORF-mediated translational regulation of mORF, polysome profiling was carried out. The results of this analysis showed that the global polysome profiles stayed unchanged (FIG. 5, panel D). Subsequent RT-qPCR analyses demonstrated that WT 5′UTR-bearing FLuc mRNA shifted to the more heavily translated fractions upon ASO1 treatment while less translatable fractions were obtained upon ASO2 treatment (FIG. 5, panel E). Inasmuch as no significant changes in mRNA levels were observed (data not shown), it was concluded ASO1 and ASO2 specifically influenced the translation efficiency of the target mRNAs. Thus, when transfected into AC16 cells, ASO1 caused cardiomyocyte (CM) hypertrophy, while ASO2 caused CM atrophy (FIG. 5, panel F).

### Example 5: Optimization and Exploration of the Utility of Type II ASO-Mediated Biomolecular Helix Formation in Broad Applications

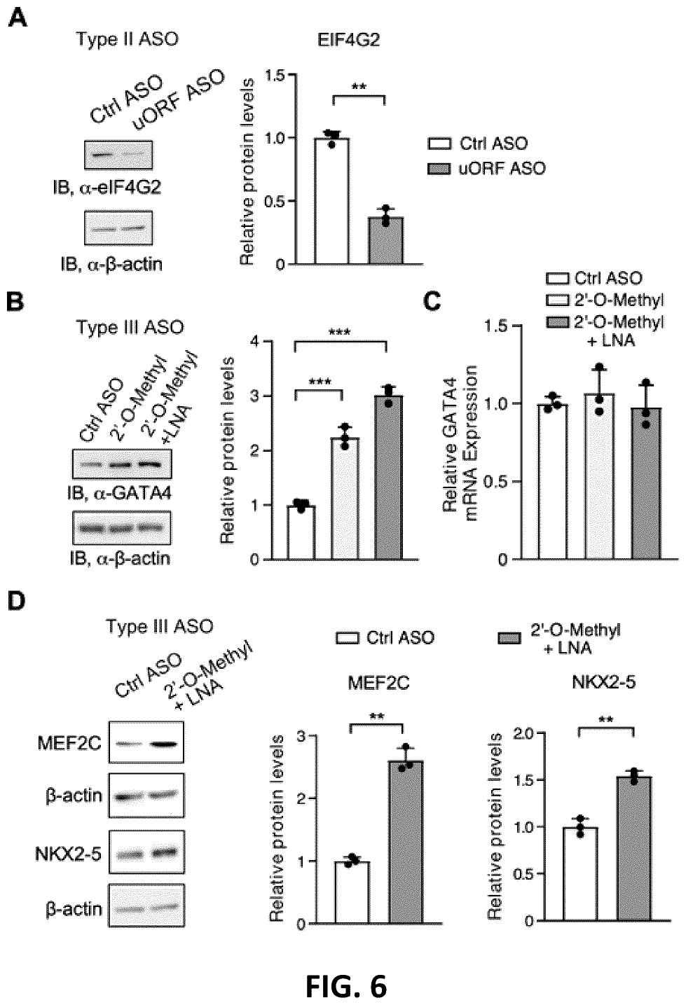

As shown in FIG. 6, Panel A, a type II uotASO targeting the uORF of the eIF4G2 mRNA result in reduced translation of eIF4G2 mORF and hence reduced amount of eIF4G2 protein as detected by Western blot. Panel B of FIG. 6 shows that type II motASOs targeting the mORF of GATA4 mRNA induced enhanced production of GATA4 protein. Specifically, the 2′-O-methyl modified type II motASO produced significant enhancement of GATA4 protein levels to 21.2±2.0% of the control ASO group (FIG. 6, Panel A). The ASOs did not alter mRNA expression (FIG. 6, Panel B). While the combination of 2′-O-methyl plus 4 LNA nucleotides at the 3′-end of the ASO resulted in an even stronger mORF-enhancing effect without affecting mRNA expression levels (FIG. 6, Panel C). Similar mORF-enhancing effect was also observed with type II motASOs targeting the mORFs in the MEF2C mRNA and NKX2-5 mRNA.

Taken together, the ASOs of the present application can decrease harmful proteins or increase beneficial ones. Additionally, the ASOs can increase protein levels in a manner that is simpler than viral delivery methods (Data not shown). Long-standing needs for overexpressing therapeutic proteins exist to treat diseases caused by genetic haploinsufficiency or pathogenic depletion. Alternatively, cell identity switch is a promising approach to improve organ function and reverse disease progressions, such as cardiac fibroblast-to-CM trans-differentiation driven by overexpressing a cocktail of TFs, including GATA4, MEF2C, TBX5, and NKX2-5. This application has provided “proof-of-concept” evidence to support the idea of increasing protein levels of GATA4, MEF2C, and NKX2-5 by ˜1.5-3 fold using the type II motASOs. Another scenario where type II motASOs can be of use is if the mRNA desired for overexpression is too large (e.g., Titin) for viral delivery approaches.

Cell identity switch is a promising approach to improve organ function and reverse disease progressions, such as cardiac fibroblast (CF)-to-cardiomyocyte (CM) trans-differentiation driven by overexpressing a cocktail of TFs, including GATA4, MEF2C, TBX5, and NKX2-5. Therefore, enhanced protein expression of GATA4, MEF2C, or NKX2-5 by type II motASOs will likely compromise cardiac fibrosis as a result of CF-to-CM transition. On the other hand, eIF4G2 promotes pro-fibrotic extracellular matrix protein translation and contributes significantly to cardiac fibrosis (unpublished results from our lab). Therefore, type II uotASO that promotes uORF activity and inhibits eIF4G2 mORF translation will reduce cardiac fibrosis.

### Example 6: CRYAB and MYBPC3 Type II motASOs Increase Protein Expression but not mRNA Expression

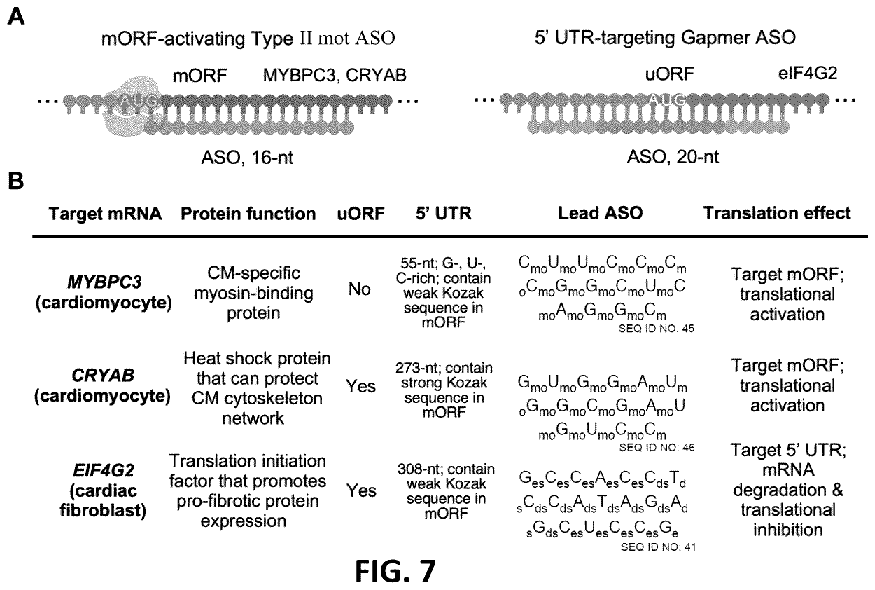

A platform has been developed whereby users can design Anti-Sense Oligonucleotides (ASOs) that bind specific regions within the main open reading frame (mORF) of mRNA and selectively increase mRNA translation and protein synthesis (Type II motASO) (FIG. 7A). This study selected two mRNA targets (MYBPC3 and CRYAB) of interest to focus on. MYBPC3 and CRYAB are myofilaments and heat shock proteins, respectively, required for normal cardiomyocyte contractile function. The proteins encoded by these two target mRNAs are known to protect the heart from cardiac fibrosis when overexpressed in cardiomyocytes (FIG. 7B).

MYBPC3 (myosin binding protein C3) encodes the cardiac isoform of myosin-binding protein C. Myosin-binding protein C is a myosin-associated protein found in the cross-bridge-bearing zone (C region) of A bands in striated muscle. MYBPC3 is expressed exclusively in the heart muscle and is a key regulator of cardiac contraction. Heterozygous mutations in this gene are a frequent cause of familial hypertrophic cardiomyopathy caused by haploinsufficiency.

CRYAB (crystallin alpha B): Mammalian lens crystallins are divided into alpha, beta, and gamma families. Alpha crystallins are composed of two gene products: alpha-A and alpha-B, for acidic and basic, respectively. Alpha crystallins can be induced by heat shock and are members of the small heat shock protein (HSP20) family. They act as molecular chaperones although they do not renature proteins and release them in the fashion of a true chaperone; instead, they hold them in large soluble aggregates. These heterogeneous aggregates consist of 30-40 subunits; the alpha-A and alpha-B subunits have a 3:1 ratio, respectively. Two additional functions of alpha crystallins are an autokinase activity and participation in the intracellular architecture. The encoded protein has been identified as a moonlighting protein based on its ability to perform mechanistically distinct functions. Alpha-A and alpha-B gene products are differentially expressed; alpha-A is preferentially restricted to the lens and alpha-B is expressed widely in many tissues and organs. Elevated expression of alpha-B crystallin occurs in many neurological diseases; a missense mutation co-segregated in a family with a desmin-related myopathy. Alternative splicing results in multiple transcript variants.

As described herein, the study designed, generated, and evaluated ASOs that have the potential to selectively increase the protein synthesis of these two targets (FIG. 7B) ((16-nt MYBPC3 ASO for mRNA translational activation using Type II motASO targeting mORF: CmoUmoUmoCmoCmoCmoCmoGmoGmoCmoUmoCmoAmoGmoGmoCm (SEQ ID NO: 45); 16-nt CRYAB ASO for mRNA translational activation using Type II motASO targeting mORF GmoUmoGmoGmoAmoUmoGmoGmoCmoGmoAmoUmoGmoUmoCmoCm (SEQ ID NO: 46)).

Bioinformatic analysis of mRNA sequence and structure was followed by designing multiple candidate ASOs targeting double-stranded RNA (dsRNA) around upstream open reading frame (uORF) and main ORF (mORF) for in vitro testing. ASOs were designed based on predicted mRNA 5′ UTR structure or 5′ UTR sequence features and then manufactured. The efficiency of candidate ASOs in manipulating protein expression of the two targets in relevant cell lines in vitro was determined. The target gene mRNA and protein expression was the regulatory readout. The translational activation effects of candidate ASOs was tested using Western blotting (measure protein steady-state level) and RT-qPCR (measure mRNA steady-state level), in AC16 human cardiomyocyte (CM; Sigma #SCC109) cell for CRYAB, MYBPC3 (cardiomycyte protection).

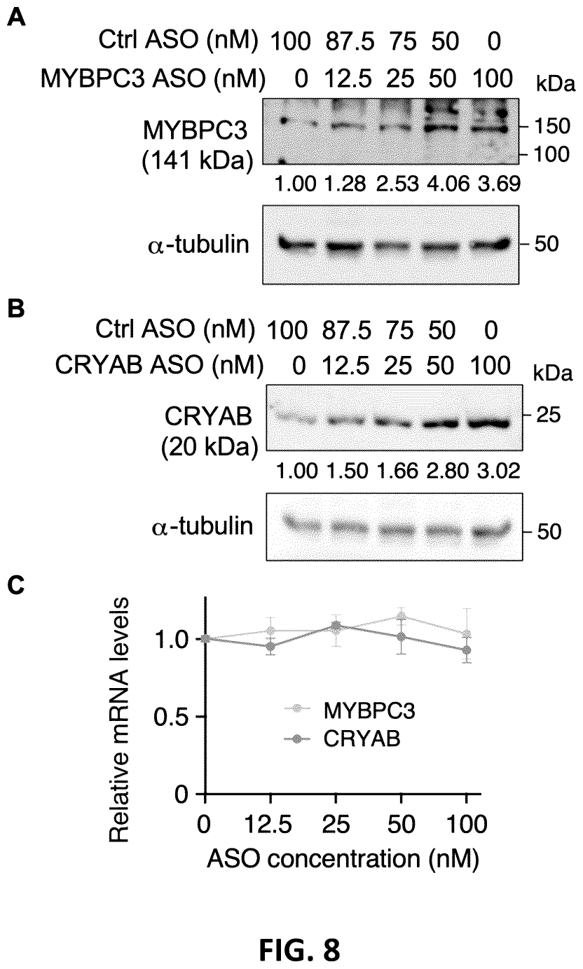

The study identified two 16-nt ASOs (with 2′-O-methyl modifications) targeting MYBPC3 and CRYAB mRNAs for translational activation. The data suggested that the two Type II motASOs can increase protein expression of MYBPC3 and CRYAB in the AC16 human cardiomyocyte cell line in a dose-dependent manner without affecting mRNA expression (FIG. 8, panels A-C).

### Example 7: 5′-UTR-Targeting Gapmer ASO Reduces eIFG4G2 Protein Expression in Immortalized Human Cardiac Fibroblasts

eIF4G2, an essential translation factor for extracellular matrix protein synthesis, has been discovered as a potential anti-fibrotic target gene in cardiac fibroblasts. A 20-nt long ASO is designed to target an evolutionarily conserved region (in humans and mice) that partially overlaps with the upstream open reading frame (uORF) with the combined feature of the Gapmer formula (10 DNA nucleic acids in the center of the ASO plus 5 RNA nucleic acids with phosphorothioate linkage) (FIG. 8, panel A and panel B, 5′-UTR-targeting Gapmer ASO, GesCesCesAesCesCdsTdsCdsCdsAdsTdsAdsGdsAdsGdsCesUesCesCesGe (SEQ ID NO:41), wherein e:MOE modification; s:phosphorothioate; d:DNA; unmodified seq: GCCACCTCCATAGAGCUCCG (SEQ ID NO: 42)=target 5′UTR of both eIF4G2 human & mouse).

Bioinformatic analysis of mRNA sequence and structure was followed by designing multiple candidate ASOs targeting double-stranded RNA (dsRNA) around upstream open reading frame (uORF) and main ORF (mORF) for in vitro testing. ASOs were designed based on predicted mRNA 5′ UTR structure or 5′ UTR sequence features and then manufactured. The efficiency of candidate ASOs in manipulating protein expression of the two targets in relevant cell lines in vitro was determined. The target gene mRNA and protein expression was the regulatory readout. The translational activation effects of candidate ASOs was tested using Western blotting (measure protein steady-state level) and RT-qPCR (measure mRNA steady-state level), in immortalized human cardiac fibroblast (IHCF; abm #T0446): eIF4G2 (anti-fibrosis effect).

One of ordinary skill in the art will recognize that the wings and the gaps discussed above may be selected and then combined in a variety of combinations to generate gapped oligomeric compounds, including, but not limited to, gapped antisense oligomeric compounds, and gapped antisense oligonucleotides. The features (length, modifications, linkages) of the 5′ wing and the 3′ wing may be selected independently of one another. The features of the gap include at least one difference in modification compared to the features of the 5′ wing and at least one difference compared to the features of the 3′ wing (i.e., there must be at least one difference in modification between neighboring regions to distinguish those neighboring regions from one another). The features of the gap may otherwise be selected independently.

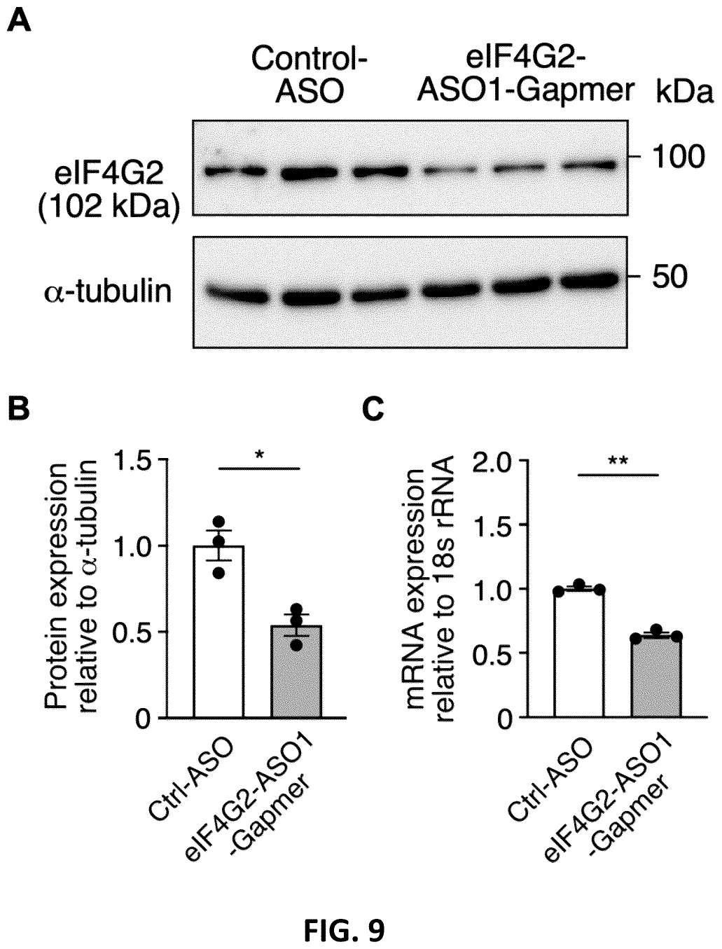

This ASO can significantly reduce EIF4G2 mRNA and protein expression via RNase H-mediated mRNA degradation (FIG. 9, panels A-C).

Taken together, these results of Examples 6 and 7 herein, show that Type II motASOs and 5′ UTR-targeting Gapmer ASOs can efficiently activate mRNA translation and silence protein expression, respectively. Using these two kinds of ASOs, users are able to either enhance the translation of anti-fibrosis mRNAs or inhibit the expression of pro-fibrotic proteins, thereby achieving potential anti-fibrotic effects in vitro or in vivo.

LIST OF SEQUENCES

| SEQ | | | |
|---|---|---|---|
| ID | | | |
| NO: | Description | Effect | Sequence |
| 1 | human GATA4 | Control for | CₘₒCₘₒAₘₒGₘₒGₘₒAₘₒUₘₒUₘₒCₘₒAₘₒAₘₒCₘₒCₘₒUₘₒ |
| | control ASO | human | AₘₒCₘ (SEQ ID NO: 1) |
| | | GATA4 | |
| | | ASOs | |
| 2 | human GATA4 | Control for | CₘₒCₘₒAₘₒGₘₒGₘₒAₘₒUₘₒUₘₒCₘₒAₘₒAₘₒCₘₒC+U+A+ |
| | control ASO | human | C (SEQ ID NO: 2) |
| | | GATA4 | |
| | | ASOs | |
| 3 | human GATA4 | Activate | AₘₒCₘₒGₘₒUₘₒAₘₒUₘₒUₘₒAₘₒAₘₒAₘₒUₘₒCₘₒCₘₒAₘₒ |
| | type II uotASO | GATA4 | GₘₒCₘ (SEQ ID NO: 3) |
| | | uORF; | |
| | | Decrease | |
| | | protein | |
| | | levels | |
| 4 | human GATA4 | Activate | AₑₒCₑₒGₑₒUₑₒAₑₒUₑₒUₑₒAₑₒAₑₒAₑₒUₑₒCₑₒCₑₒAₑₒGₑₒCₑ |
| | type II uotASO | GATA4 | (SEQ ID NO: 4) |
| | | uORF; | |
| | | Decrease | |
| | | protein | |
| | | levels | |
| 5 | human GATA4 | Activate | +A+C+G+U+A+U+U+A+A+A+U+C+C+A+G+C |
| | type II uotASO | GATA4 | (SEQ ID NO: 5) |
| | | uORF; | |
| | | Decrease | |
| | | protein | |
| | | levels | |
| 6 | human GATA4 | Activate | GₘₛCₘₛUₘₛGₘₒAₘₒAₘₒUₘₒUₘₒCₘₒAₘₒCₘₒCₘₒCₘₛCₘₛAₘₛ |
| | type II uotASO | GATA4 | Gₘ (SEQ ID NO: 6) |
| | | uORF; | |
| | | Decrease | |
| | | protein | |
| | | levels | |
| 7 | human GATA4 | Activate | AₘₒCₘₒGₘₒUₘₒAₘₒUₘₒUₘₒAₘₒAₘₒAₘₒUₘₒCₘₒ+C+A+G+C |
| | type II uotASO | GATA4 | (SEQ ID NO: 7) |
| | | uORF; | |
| | | Decrease | |
| | | protein | |
| | | levels | |
| 8 | human GATA4 | Inhibit | GₘₒCₘₒUₘₒGₘₒAₘₒAₘₒUₘₒUₘₒCₘₒAₘₒCₘₒCₘₒCₘₒCₘₒ |
| | type I uotASO | GATA4 | AₘₒGₘ (SEQ ID NO: 8) |
| | | uORF; | |
| | | Increase | |
| | | protein | |
| | | levels | |
| 9 | human GATA4 | Activate | GₘₒGₘₒCₘₒCₘₒAₘₒAₘₒGₘₒCₘₒUₘₒCₘₒUₘₒGₘₒAₘₒUₘₒ |
| | type II ₘₒtASO | GATA4 | AₘₒCₘ (SEQ ID NO: 9) |
| | | mORF; | |
| | | Increase | |
| | | protein | |
| | | levels | |
| 10 | human GATA4 | Activate | GₘₒGₘₒCₘₒCₘₒAₘₒAₘₒGₘₒCₘₒUₘₒCₘₒUₘₒGₘₒ+A+T+A+C |
| | type II ₘₒtASO | GATA4 | (SEQ ID NO: 10) |
| | | mORF; | |
| | | Increase | |
| | | protein | |
| | | levels | |
| 11 | mouse GATA4 | Control for | CₘₒCₘₒAₘₒGₘₒGₘₒAₘₒUₘₒUₘₒCₘₒAₘₒAₘₒCₘₒCₘₒUₘₒAₘₒ |
| | control ASO | Mouse | Cₘ (SEQ ID NO: 11) |
| | | GATA4 | |
| | | ASO | |
| 12 | mouse GATA4 | Activate | AₘₒCₘₒGₘₒAₘₒAₘₒUₘₒUₘₒAₘₒAₘₒAₘₒUₘₒCₘₒCₘₒAₘₒGₘₒ |
| | type II uotASO | Gata4 | Cₘ (SEQ ID NO: 12) |
| | | uORF; | |
| | | Decrease | |
| | | protein | |
| | | levels | |
| 13 | human NKX2-5 | Control for | CₘₒAₘₒGₘₒUₘₒCₘₒAₘₒAₘₒCₘₒUₘₒCₘₒCₘₒU+C+T+T+C |
| | control ASO | human | (SEQ ID NO: 13) |
| | | NKX2-5 | |
| | | ASOs | |
| 14 | human NKX2-5 | Activate | CₘₒUₘₒGₘₒGₘₒCₘₒAₘₒGₘₒCₘₒUₘₒUₘₒCₘₒC+C+T+G+C |
| | type II uotASO | NKX2-5 | (SEQ ID NO: 14) |
| | | uORF; | |
| | | Decrease | |
| | | protein | |
| | | levels | |
| 15 | human NKX2-5 | Activate | AₘₒGₘₒCₘₒAₘₒGₘₒGₘₒGₘₒCₘₒUₘₒGₘₒGₘₒGₘₒGₘₒAₘₒ |
| | type II ₘₒtASO | NKX2-5 | AₘₒCₘ (SEQ ID NO: 15) |
| | | mORF; | |
| | | Increase | |
| | | protein | |
| | | levels | |
| 16 | human eIF4G2 | Control for | UₘₒGₘₒCₘₒAₘₒGₘₒUₘₒCₘₒGₘₒCₘₒCₘₒUₘₒCₘₒ+C+T+C+C |
| | control ASO | human | (SEQ ID NO: 16) |
| | | EIF4G2 | |
| | | ASOs | |
| 17 | human eIF4G2 | Activate | AₘₒCₘₒCₘₒCₘₒGₘₒCₘₒUₘₒGₘₒCₘₒCₘₒAₘₒC+C+T+C+C |
| | type II uotASO | EIF4G2 | (SEQ ID NO: 17) |
| | | uORF; | |
| | | Decrease | |
| | | protein | |
| | | levels | |
| 18 | human eIF4G2 | Activate | UₘₒGₘₒCₘₒAₘₒAₘₒUₘₒCₘₒGₘₒCₘₒAₘₒCₘₒU+C+T+C+C |
| | type II motASO | EIF4G2 | (SEQ ID NO: 18) |
| | | mORF; | |
| | | Increase | |
| | | protein | |
| | | levels | |
| 19 | human MEF2C | Control for | AₘₒGₘₒUₘₒGₘₒCₘₒAₘₒAₘₒUₘₒAₘₒCₘₒUₘₒC+A+T+G+ |
| | control ASO | human | C (SEQ ID NO: 19) |
| | | MEF2C | |
| | | ASOs | |
| 20 | human MEF2C | Activate | GₘₒUₘₒUₘₒC ₘₒCₘₒCₘₒAₘₒAₘₒAₘₒUₘₒUₘₒC+C+T+G+ |
| | type II uotASO | MEF2C | C (SEQ ID NO: 20) |
| | | uORF; | |
| | | Decrease | |
| | | protein | |
| | | levels | |
| 21 | human MEF2C | Activate | AₘₒAₘₒUₘₒC ₘₒU ₘₒUₘₒUₘₒUₘₒUₘₒUₘₒCₘₒU+C+C+C+ |
| | type II motASO | MEF2C | C |
| | | mORF; | (SEQ ID NO: 21) |
| | | Increase | |
| | | protein | |
| | | levels | |
| 22 | human GATA4 | | ggggacuugg aggcggccgg cgcaggggcc gcgagaggcu ucgucgccgc |
| | mRNA | | ugcagcuccg |
| | uORF sequence: | | ggggcuccca ggggagcgug cgcggaaccu ccaggcccag caggaccccg |
| | 382-411 (30-nt) | | gcugcggcga |
| | mORF | | ggaggaagga gccagccuag cagcuucugc gccuguggcc gcgggugucc |
| | sequence: 561- | | uggaggccuc |
| | 1892 (1332-nt) | | ucggugugac gaguggggga cccgaaggcu cgugcgccac cuccaggccu |
| | | | ggacgcugcc |
| | | | cuccgucuuc ugcccccaau aggugcgccg gaccuucagg cccuggggug |
| | | | aauucagcug |
| | | | cuccuacauc agcuuccgga accaccaaaa auucaaauug ggauuuuccg |
| | | | gaguaaacaa |
| | | | gagccuagag cccuuugcuc aaugcuggau uuaauacgua uauauuuuua |
| | | | agcgaguugg |
| | | | uuuuuucccc uuugauuuuu gaucuucgcg acaguuccuc ccacgcauau |
| | | | uaucguuguu |
| | | | gccgucguuu ucucuccccg cguggcuccu ugaccugcga gggagagaga |
| | | | ggacaccgaa |
| | | | gccgggagcu cgcagggacc auguaucaga gcuuggccau ggccgccaac |
| | | | cacgggccgc |
| | | | cccccggugc cuacgaggcg ggcggccccg gcgccuucau gcacggcgcg |
| | | | ggcgccgcgu |
| | | | ccucgccagu cuacgugcce acaccgcggg ugcccuccuc cgugcugggc |
| | | | cuguccuacc |
| | | | uccagggcgg aggcgcgggc ucugcguccg gaggcgccuc gggcggcagc |
| | | | uccggugggg |
| | | | ccgcgucugg ugcggggccc gggacccagc agggcagccc gggauggagc |
| | | | caggcgggag |
| | | | ccgacggagc cgcuuacacc ccgccgccgg ugucgccgcg cuucuccuuc |
| | | | ccggggacca |
| | | | ccgggucccu ggcggccgcc gccgccgcug ccgcggcccg ggaagcugcg |
| | | | gccuacagca |
| | | | guggcggcgg agcggcgggu gcgggccugg cgggccgcga gcaguacggg |
| | | | cgcgccggcu |
| | | | ucgcgggcuc cuacuccagc cccuacccgg cuuacauggc cgacgugggc |
| | | | gcguccuggg |
| | | | ccgcagccgc cgccgccucc gccggccccu ucgacagcce gguccugcac |
| | | | agccugcccg |
| | | | gccgggccaa cccggccgcc cgacacccca aucucguaga uauguuugac |
| | | | gacuucucag |
| | | | aaggcagaga gugugucaac uguggggcua uguccacccc gcucuggagg |
| | | | cgagauggga |
| | | | cgggucacua ucugugcaac gccugcggcc ucuaccacaa gaugaacggc |
| | | | aucaaccggc |
| | | | cgcucaucaa gccucagcgc cggcuguccg ccucccgccg agugggccuc |
| | | | uccugugcca |
| | | | acugccagac caccaccacc acgcuguggc gccgcaaugc ggagggcgag |
| | | | ccugugugca |
| | | | augccugcgg ccucuacaug aagcuccacg ggguccccag gccucuugca |
| | | | augcggaaag |
| | | | aggggaucca aaccagaaaa cggaagccca agaaccugaa uaaaucuaag |
| | | | acaccagcag |
| | | | cuccuucagg cagugagagc cuuccucccg ccagcggugc uuccagcaac |
| | | | uccagcaacg |
| | | | ccaccaccag cagcagcgag gagaugcguc ccaucaagac ggagccuggc |
| | | | cugucaucuc |
| | | | acuacgggca cagcagcucc gugucccaga cguucucagu cagugcgaug |
| | | | ucuggccaug |
| | | | ggcccuccau ccacccuguc cucucggccc ugaagcucuc cccacaaggc |
| | | | uaugcgucuc |
| | | | ccgucagcca gucuccacag accagcucca agcaggacuc uuggaacagc |
| | | | cuggucuugg |
| | | | ccgacaguca cggggacaua aucacugcgu aa |
| 23 | mouse Gata4 | | gcgccgggag caggggacaa gccggaggcc cgcagagugg ccgcccgagg |
| | mRNA | | cucagccgca |
| | uORF: 426-482 | | guugcagcuc cgcggacuca cggagaucgc gccgguuuuc ugggaaacug |
| | (57-nt) | | gagcuggcca |
| | mORF: 619- | | ggacugccgc uucgcuucga agggaccggg cccucuuugu cauucuucgc |
| | 1947 (1329-nt) | | uggagccgcu |
| | | | cuggagcuag cagcugcgcc ugggugugua gcaggcagaa agcaaggacu |
| | | | aggcuucuuu |
| | | | agccgguggg ugauccgaag gccugcucag gguguucgag accagccugg |
| | | | acugcgucug |
| | | | ggcaccucca gccucugggc ccuggaauag aguccgcccu cccgcacgau |
| | | | uucuggagca |
| | | | accgcaaauc caauuuggga uuuucuuuuu ccugagcaaa ccagagccua |
| | | | gucugccucg |
| | | | ugcucagagc uuggggcgau guaccaaagc cuggccaugg ccgccaacca |
| | | | cggccccccg |
| | | | cccggcgccu acgaagcagg uggcccuggc gccuucaugc acagcgcggg |
| | | | cgccgcgucc |
| | | | ucgcccgucu acgugcccac uccgcgggug ccguccucug ugcugggccu |
| | | | guccuaccug |
| | | | cagggcggug gcagugccgc ugcagcugga accaccucgg guggcagcuc |
| | | | cggggccggc |
| | | | ccgucgggug cagggccugg gacccagcag gguagcccug gcuggagcca |
| | | | agcuggagcc |
| | | | gagggagccg ccuacacccc gccgcccgug uccccgcgcu ucucuuuccc |
| | | | ggggacuacu |
| | | | gggucccugg cggccgcugc cgccgcugcc gcagcccggg aagcugcagc |
| | | | cuacggcagu |
| | | | ggcggcgggg cggcgggcgc uggucuggcu ggccgagagc aguacgggcg |
| | | | uccgggcuuc |
| | | | gccggcuccu acuccagcce cuacccagcc uacauggccg acgugggagc |
| | | | auccugggcc |
| | | | gcagccgcug ccgccucugc cggccccuuc gacagcccag uccugcacag |
| | | | ccugccugga |
| | | | cgggccaacc cuggaagaca ccccaaucuc guagauaugu uugaugacuu |
| | | | cucagaaggc |
| | | | agagagugug ucaauugugg ggccaugucc accccacucu ggaggcgaga |
| | | | ugggacggga |
| | | | cacuaccugu gcaaugccug uggccucuau cacaagauga acggcaucaa |
| | | | ccggccccuc |
| | | | auuaagccuc agcgccgccu guccgcuucc cgccggguag gccucuccug |
| | | | ugccaacugc |
| | | | cagacuacca ccaccacgcu guggcgucgu aaugccgagg gugagccugu |
| | | | auguaaugcc |
| | | | ugcggccucu acaugaagcu ccaugggguu cccaggccuc uugcaaugcg |
| | | | gaaggagggg |
| | | | auucaaacca gaaaacggaa gcccaagaac cugaauaaau cuaagacgcc |
| | | | agcagguccu |
| | | | gcuggugaga cccucccucc cuccaguggu gccuccagcg guaacuccag |
| | | | caaugccacu |
| | | | agcagcagca gcagcaguga agagaugcgc cccaucaaga cagagcccgg |
| | | | gcugucaucu |
| | | | cacuaugggc acagcagcuc caugucccag acauucagua cuguguccgg |
| | | | ccacgggccc |
| | | | uccauccauc cagugcuguc ugcucugaag cuguccccac aaggcuaugc |
| | | | aucuccuguc |
| | | | acucagacau cgcaggccag cuccaagcag gacucuugga acagccuggu |
| | | | ccuggcugac |
| | | | agucaugggg acauaaucac cgcguaa |
| 24 | human MEF2C | | gcagucacag acacuugagc acacgcguac acccagacau cuucgggcug |
| | mRNA | | cuauuggauu |
| | uORF sequence: | | gacuuugaag guucugugug ggucgccgug gcugcauguu ugaaucaggu |
| | uORF1: 96-140 | | ggagaagcac |
| | (45-nt); uORF2: | | uucaacgcug gacgaaguaa agauuauugu uguuauuuuu uuuuucucuc |
| | 240-260 (21-nt) | | ucucucucuc |
| | mORF | | uuaagaaagg aaaauauccc aaggacuaau cugaucgggu cuuccuucau |
| | sequence: 373- | | caggaacgaa |
| | 1794 (1422-nt) | | ugcaggaauu ugggaacuga gcugugcaag ugcugaagaa ggagauuugu |
| | | | uuggaggaaa |
| | | | caggaaagag aaagaaaagg aaggaaaaaa uacauaauuu cagggacgag |
| | | | agagagaaga |
| | | | aaaacgggga cuauggggag aaaaaagauu cagauuacga ggauuaugga |
| | | | ugaacguaac |
| | | | agacagguga cauuuacaaa gaggaaauuu ggguugauga agaaggcuua |
| | | | ugagcugagc |
| | | | gugcugugug acugugagau ugcgcugauc aucuucaaca gcaccaacaa |
| | | | gcuguuccag |
| | | | uaugccagca ccgacaugga caaagugcuu cucaaguaca cggaguacaa |
| | | | cgagccgcau |
| | | | gagagccgga caaacucaga caucguggag acguugagaa agaagggccu |
| | | | uaauggcugu |
| | | | gacagcccag accccgaugc ggacgauucc guaggucaca gcccugaguc |
| | | | ugaggacaag |
| | | | uacaggaaaa uuaacgaaga uauugaucua augaucagca ggcaaagauu |
| | | | gugugcuguu |
| | | | ccaccuccca acuucgagau gccagucucc aucccagugu ccagccacaa |
| | | | caguuuggug |
| | | | uacagcaacc cugucagcuc acugggaaac cccaaccuau ugccacuggc |
| | | | ucacccuucu |
| | | | cugcagagga auaguauguc uccuggugua acacaucgac cuccaagugc |
| | | | agguaacaca |
| | | | gguggucuga uggguggaga ccucacgucu ggugcaggca ccagugcagg |
| | | | gaacggguau |
| | | | ggcaaucccc gaaacucacc aggucugcug gucucaccug guaacuugaa |
| | | | caagaauaug |
| | | | caagcaaaau cuccuccccc aaugaauuua ggaaugaaua accguaaacc |
| | | | agaucuccga |
| | | | guucuuauuc caccaggcag caagaauacg augccaucag ugucugagga |
| | | | ugucgaccug |
| | | | cuuuugaauc aaaggauaaa uaacucccag ucggcucagu cauuggcuac |
| | | | cccagugguu |
| | | | uccguagcaa cuccuacuuu accaggacaa ggaaugggag gauauccauc |
| | | | agccauuuca |
| | | | acaacauaug guaccgagua cucucugagu agugcagacc ugucaucucu |
| | | | gucuggguuu |
| | | | aacaccgcca gcgcucuuca ccuugguuca guaacuggcu ggcaacagca |
| | | | acaccuacau |
| | | | aacaugccac caucugcccu cagucaguug ggagcuugca cuagcacuca |
| | | | uuuaucucag |
| | | | aguucaaauc ucucccugcc uucuacucaa agccucaaca ucaagucaga |
| | | | accuguuucu |
| | | | ccuccuagag accguaccac caccccuucg agauacccac aacacacgcg |
| | | | ccacgaggcg |
| | | | gggagaucuc cuguugacag cuugagcagc uguagcaguu cguacgacgg |
| | | | gagcgaccga |
| | | | gaggaucacc ggaacgaauu ccacuccccc auuggacuca ccagaccuuc |
| | | | gccggacgaa |
| | | | agggaaaguc ccucagucaa gcgcaugcga cuuucugaag gaugggcaac auga |
| 25 | human NKX2-5 | | acauccagag cuggccgacg ggugcgcggg cgggcggcgg caccaugcag |
| | mRNA | | ggaagcugcc |
| | uORF sequence: | | aggggccgug ggcagcgccg cuuucugccg cccaccuggc gcugugagac |
| | 45-107 (63-nt) | | uggcgcugcc |
| | mORF | | accauguucc ccagcccugc ucucacgcce acgcccuucu cagucaaaga |
| | sequence: 124- | | cauccuaaac |
| | 1098 (975-nt) | | cuggaacagc agcagcgcag ccuggcugcc gccggagagc ucucugcccg |
| | | | ccuggaggcg |
| | | | acccuggcgc ccuccuccug caugcuggcc gccuucaagc cagaggccua |
| | | | cgcugggccc |
| | | | gaggcggcug cgccgggccu cccagagcug cgcgcagagc ugggccgcgc |
| | | | gccuucaccg |
| | | | gccaagugug cgucugccuu ucccgccgcc cccgccuucu auccacgugc |
| | | | cuacagcgac |
| | | | cccgacccag ccaaggaccc uagagccgaa aagaaagagc ugugcgcgcu |
| | | | gcagaaggcg |
| | | | guggagcugg agaagacaga ggcggacaac gcggagcggc cccgggcgcg |
| | | | acggcggagg |
| | | | aagccgcgcg ugcucuucuc gcaggcgcag gucuaugagc uggagcggg |
| | | | cuucaagcag |
| | | | cagcgguacc ugucggcccc cgaacgcgac cagcuggcca gcgugcugaa |
| | | | acucacgucc |
| | | | acgcagguca agaucugguu ccagaaccgg cgcuacaagu gcaagcggca |
| | | | gcggcaggac |
| | | | cagacucugg agcugguggg gcugcccccg ccgccgccgc cgccugcccg |
| | | | caggaucgcg |
| | | | gugccagugc uggugcgcga uggcaagcca ugccuagggg acucggcgcc |
| | | | cuacgcgccu |
| | | | gccuacggcg ugggccucaa ucccuacggu uauaacgccu accccgccua |
| | | | uccggguuac |
| | | | ggcggcgcgg ccugcagcce uggcuacagc ugcacugccg cuuaccccgc |
| | | | cgggccuucc |
| | | | ccagcgcagc cggccacugc cgccgccaac aacaacuucg ugaacuucgg |
| | | | cgucggggac |
| | | | uugaaugcgg uucagagcce cgggauuccg cagagcaacu cgggaguguc |
| | | | cacgcugcau |
| | | | gguauccgag ccugguag |
| 26 | human eIF4G2 | | gccagcagug agucggagcu cuauggaggu ggcagcgggu accgaguggc |
| | mRNA | | ggcugcagca |
| | (isoform 1) | | gcgacuccuc ugagugcagu uugaggccgu ccccgacucc uuccuccccc |
| | uORF sequence: | | uucccucccc |
| | 23-73 (51-nt) | | cuuuuuuuug uuuuccguuc cccuuucccc ucccuucccu auccccgacg |
| | mORF | | accggauccu |
| | sequence: 309- | | gaggaggcag cugcgguggc agcugcugag uucucgguga agguauuuca |
| | 3032 (2724-nt) | | uuucuccugu |
| | | | ccccuccccu ccccacccca ucuauuaaua uuauucuuuu gaagauucuu |
| | | | cguugucaag |
| | | | ccgccaaagu ggagagugcg auugcagaag ggggugcuuc ucguuucagu |
| | | | gcuucuucgg |
| | | | gcggaggagg aaguaggggu gcaccucagc acuaucccaa gacugcuggc |
| | | | aacagcgagu |
| | | | uccuggggaa aaccccaggg caaaacgcuc agaaauggau uccugcacga |
| | | | agcacuagac |
| | | | gagaugacaa cuccgcagca aacaacuccg caaacgaaaa agaacgacau |
| | | | gaugcaaucu |
| | | | ucaggaaagu aagaggcaua cuaaauaagc uuacuccuga aaaguuugac |
| | | | aagcuaugcc |
| | | | uugagcuccu caaugugggu guagagucua aacucauccu uaaagggguc |
| | | | auacugcuga |
| | | | uuguggacaa agcccuagaa gagccaaagu auagcucacu guaugcucag |
| | | | cuaugucugc |
| | | | gauuggcaga agaugcacca aacuuugaug gcccagcagc agagggucaa |
| | | | ccaggacaga |
| | | | agcaaagcac cacauucaga cgccuccuaa uuuccaaauu acaagaugaa |
| | | | uuugaaaacc |
| | | | gaacuagaaa uguugaugue uaugauaagc gugaaaauce ccuccucccc |
| | | | gaggaggagg |
| | | | aacagagagc cauugcuaag aucaagaugu ugggaaacau caaauucauu |
| | | | ggagagcuug |
| | | | gcaagcuuga ucuuauucac gaaucuauce uucauaagug caucaaaaca |
| | | | cuuuuggaaa |
| | | | agaagaagag aguccaacuc aaagauaugg gagaggauuu ggagugccuc |
| | | | ugucagauaa |
| | | | ugaggacagu gggaccuaga uuagaccaug aacgagccaa guccuuaaug |
| | | | gaucaguacu |
| | | | uugcccgaau gugcuccuug auguuaagua aggaauugce agcaaggauu |
| | | | cguuuccugc |
| | | | ugcaggauac cguagaguug cgagaacace auuggguucc ucgcaaggcu |
| | | | uuucuugaca |
| | | | auggaccaaa gacgaucaau caaauucguc aagaugcagu aaaagaucua |
| | | | ggguguuuua |
| | | | uuccugcucc uauggcucaa gggaugagaa gugacuucuu ucuggaggga |
| | | | ccguucaugc |
| | | | cacccaggau gaaaauggau agggacccac uuggaggacu ugcugauaug |
| | | | uuuggacaaa |
| | | | ugccagguag cggaauuggu acugguccag gaguuaucca ggauagauuu |
| | | | ucacccacca |
| | | | ugggacguca ucguucaaau caacucuuca auggccaugg gggacacauc |
| | | | augccuccca |
| | | | cacaaucgca guuuggagag augggaggca aguuuaugaa aagccagggg |
| | | | cuaagccagc |
| | | | ucuaccauaa ccagagucag ggacucuuau cccagcugca aggacagucg |
| | | | aaggauaugc |
| | | | caccucgguu uucuaagaaa ggacagcuua augcagauga gauuagccug |
| | | | aggccugcuc |
| | | | agucguuccu aaugaauaaa aaucaagugc caaagcuuca gccccagaua |
| | | | acuaugauuc |
| | | | cuccuagugc acaaccacca cgcacucaaa caccaccucu gggacagaca |
| | | | ccucagcuug |
| | | | gucucaaaac uaauccaccg cuuauccagg aaaagccugc caagaccagc |
| | | | aaaaagccac |
| | | | caccgucaaa ggaagaacuc cuuaaacuaa cugaaacugu ugugacugaa |
| | | | uaucuaaaua |
| | | | guggaaaugc aaaugaggcu gucaauggug uaagagaaau gagggcuccu |
| | | | aaacacuuuc |
| | | | uuccugagau guuaagcaaa guaaucaucc ugucacuaga uagaagcgau |
| | | | gaagauaaag |
| | | | aaaaagcaag uucuuugauc aguuuacuca aacaggaagg gauagccaca |
| | | | agugacaacu |
| | | | ucaugcaggc uuuccugaau guauuggacc agugucccaa acuggagguu |
| | | | gacaucccuu |
| | | | uggugaaauc cuauuuagca caguuugcag cucgugccau cauuucagag |
| | | | cuggugagca |
| | | | uuucagaacu agcucaacca cuagaaagug gcacccauuu uccucucuuc |
| | | | cuacuuuguc |
| | | | uucagcaguu agcuaaauua caagaucgag aaugguuaac agaacuuuuu |
| | | | caacaaagca |
| | | | aggucaauau gcagaaaaug cucccagaaa uugaucagaa uaaggaccgc |
| | | | auguuggaga |
| | | | uuuuggaagg aaagggacug aguuucuuau ucccacuccu caaauuggag |
| | | | aaggaacugu |
| | | | ugaagcaaau aaaguuggau ccauccccuc aaaccauaua uaaauggauu |
| | | | aaagauaaca |
| | | | ucucucccaa acuucaugua gauaaaggau uugugaacau cuuaaugacu |
| | | | agcuucuuac |
| | | | aguacauuuc uagugaagua aaccccccca gcgaugaaac agauucaucc |
| | | | ucugcuccuu |
| | | | ccaaagaaca guuagagcag gaaaaacaac uacuacuauc uuucaagcca |
| | | | guaaugcaga |
| | | | aauuucuuca ugaucacguu gaucuacaag ucagugcccu guaugcucuc |
| | | | caggugcacu |
| | | | gcuauaacag caacuuccca aaaggcaugu uacuucgcuu uuuugugcac |
| | | | uucuaugaca |
| | | | uggaaauuau ugaagaagaa gcuuucuugg cuuggaaaga agauauaacc |
| | | | caagaguuuc |
| | | | cgggaaaagg caaggcuuug uuccagguga aucaguggcu aaccugguua |
| | | | gaaacugcug |
| | | | aagaagaaga aucagaggaa gaagcugacu aa |
| 27 | human GATA4 | | cuggggugaauucagc |
| | type I uotASO | | |
| | targct sequence | | |
| 28 | human GATA4 | | guaucagagcuuggcc |
| | type II motASO | | |
| | targct sequence | | |
| 29 | human MEF2C | | ggggagaaaaaagauu |
| | type II motASO | | |
| | targct sequence | | |
| 30 | human NKX2-5 | | guuccccagcccugcu |
| | type II motASO | | |
| | targct sequence | | |
| 31 | human eIF4G2 | | ggagguggcagcgggt |
| | type II uotASO | | |
| | targct sequence | | |
| 32 | human, gorilla | | ATGCTGGATTTAATACGTATATATTTTTAA |
| | and monkey | | |
| | GATA4 uORF | | |
| | DNA sequence | | |
| 33 | cat and dolphin | | ATGCTGGATTTAATTCGTATATATTTTTGA |
| | GATA4 uORF | | |
| | DNA sequence | | |
| 34 | golden hamster | | ATGCTGGATTTAATTCGCATATATTTTGAGCGA |
| | GATA4 uORF | | GTTGGGCCTCTCCCCATCGATTTTTGA |
| | DNA sequence | | |
| 35 | rat GATA4 | | ATGCTGGATTTAATTCGTATATATTTTGAGCGA |
| | uORF DNA | | GTTGGGCCTGTCCTCATTGTTTTTTGA |
| | sequence | | |
| 36 | mouse GATA4 | | ATGCTGGATTTAATTCGTATATATTTTGAGCGA |
| | uORF DNA | | GTTGGGCCTCTCCTCGTTTTTTGA |
| | sequence | | |
| 37 | WT human | | GGGGTGAATTCAGCTGCTCCTACATCAGCTTCC |
| | GATA4 stem | | GGAACCACCAAAAATTCAAATTGGGATTTTCCG |
| | loop rcgion | | GAGTAAACAAGAGCCTAGAGCCCTTTGCTCAAT |
| | | | GCTGGATTTAATA |
| 38 | AuORF human | | GGGGTGAATTCAGCTGCTCCTACATCAGCTTCC |
| | GATA4 stem | | GGAACCACCAAAAATTCAAATTGGGATTTTCCG |
| | loop rcgion | | GAGTAAACAAGAGCCTAGAGCCCTTTGCTCATT |
| | | | GCTGGATTTAATA |
| 39 | Mut human | | GGGGTGACTTAAGCTGCTCCTACATCAGCTTCC |
| | GATA4 stem | | GGAACCACCAAAAATTCAAATTGGGATTTTCCG |
| | loop rcgion | | GAGTAAACAAGAGCCTAGAGCCCTTTGCTCAAT |
| | | | GCTGGATTTAATA |
| 40 | Rescue Mut | | GGGGTGACTTAAGCTGCTCCTACATCAGCTTCC |
| | human GATA4 | | GGAACCACCAAAAATTCAAATTGGGATTTTCCG |
| | stem loop region | | GAGTAAACAAGAGCCTAGAGCCCTTTGCTCAAT |
| | | | GCTTGAGTTAATA |
| 41 | human eIF4G2 | Inhibit | GesCesCesAesCesCdsTdsCdsCdsAdsTdsAdsGdsAdsG |
| | 5′-UTR | eIF4G2 | dsCesUesCesCesGe |
| | targcting | mORF; | |
| | gapmer ASO | Decrease | |
| | | protein | |
| | | levels | |
| 42 | unmodified | | GCCACCTCCATAGAGCUCCG |
| | EIF4G2-ASO | | |
| 43 | human | | agtccctctt tgggtgacct gtgcctgctt cgtgcctggt gtgacgtctc tcaggatgcc |
| | MYBPC3 | | tgagccgggg aagaagccag tctcagcttt tagcaagaag ccacggtcag |
| | mRNA | | tggaagtggc |
| | 5′ UTR: 1-55 | | cgcaggcagc cctgccgtgt tcgaggccga gacagagcgg gcaggagtga |
| | (55-nt) | | aggtgcgctg |
| | mORF | | gcagcgcgga ggcagtgaca tcagcgccag caacaagtac ggcctggcca |
| | sequence: 56- | | cagagggcac |
| | 3880 (3825-nt) | | acggcatacg ctgacagtgc gggaagtggg ccctgccgac cagggatctt |
| | | | acgcagtcat |
| | | | tgctggctcc tccaaggtca agttcgacct caaggtcata gaggcagaga |
| | | | aggcagagcc |
| | | | catgctggcc cctgcccctg cccctgctga ggccactgga gcccctggag |
| | | | aagccccggc |
| | | | cccagccgct gagctgggag aaagtgcccc aagtcccaaa gggtcaagct |
| | | | cagcagctct |
| | | | caatggtcct acccctggag cccccgatga ccccattggc ctcttcgtga tgcggccaca |
| | | | ggatggcgag gtgaccgtgg gtggcagcat caccttctca gcccgcgtgg |
| | | | ccggcgccag |
| | | | cctcctgaag ccgcctgtgg tcaagtggtt caagggcaaa tgggtggacc |
| | | | tgagcagcaa |
| | | | ggtgggccag cacctgcagc tgcacgacag ctacgaccgc gccagcaagg |
| | | | tctatctgtt |
| | | | cgagctgcac atcaccgatg cccagcctgc cttcactggc agctaccgct gtgaggtgtc |
| | | | caccaaggac aaatttgact gctccaactt caatctcact gtccacgagg ccatgggcac |
| | | | cggagacctg gacctcctat cagccttccg ccgcacgagc ctggctggag |
| | | | gtggtcggcg |
| | | | gatcagtgat agccatgagg acactgggat tctggacttc agctcactgc tgaaaaagag |
| | | | agacagtttc cggaccccga gggactcgaa gctggaggca ccagcagagg |
| | | | aggacgtgtg |
| | | | ggagatccta cggcaggcac ccccatctga gtacgagcgc atcgccttcc |
| | | | agtacggcgt |
| | | | cactgacctg cgcggcatgc taaagaggct caagggcatg aggcgcgatg |
| | | | agaagaagag |
| | | | cacagccttt cagaagaagc tggagccggc ctaccaggtg agcaaaggcc |
| | | | acaagatccg |
| | | | gctgaccgtg gaactggctg accatgacgc tgaggtcaaa tggctcaaga |
| | | | atggccagga |
| | | | gatccagatg agcggcagca agtacatctt tgagtccatc ggtgccaagc gtaccctgac |
| | | | catcagccag tgctcattgg cggacgacgc agcctaccag tgcgtggtgg |
| | | | gtggcgagaa |
| | | | gtgtagcacg gagctctttg tgaaagagcc ccctgtgctc atcacgcgcc ccttggagga |
| | | | ccagctggtg atggtggggc agcgggtgga gtttgagtgt gaagtatcgg |
| | | | aggagggggc |
| | | | gcaagtcaaa tggctgaagg acggggtgga gctgacccgg gaggagacct |
| | | | tcaaataccg |
| | | | gttcaagaag gacgggcaga gacaccacct gatcatcaac gaggccatgc |
| | | | tggaggacgc |
| | | | ggggcactat gcactgtgca ctagcggggg ccaggcgctg gctgagctca |
| | | | ttgtgcagga |
| | | | aaagaagctg gaggtgtacc agagcatcgc agacctgatg gtgggcgcaa |
| | | | aggaccaggc |
| | | | ggtgttcaaa tgtgaggtct cagatgagaa tgttcggggt gtgtggctga agaatgggaa |
| | | | ggagctggtg cccgacagcc gcataaaggt gtcccacatc ggggggtcc |
| | | | acaaactgac |
| | | | cattgacgac gtcacacctg ccgacgaggc tgactacagc tttgtgcccg agggcttcgc |
| | | | ctgcaacctg tcagccaagc tccacttcat ggaggtcaag attgacttcg tacccaggca |
| | | | ggaacctccc aagatccacc tggactgccc aggccgcata ccagacacca ttgtggttgt |
| | | | agctggaaat aagctacgtc tggacgtccc tatctctggg gaccctgctc ccactgtgat |
| | | | ctggcagaag gctatcacgc aggggaataa ggccccagcc aggccagccc |
| | | | cagatgcccc |
| | | | agaggacaca ggtgacagcg atgagtgggt gtttgacaag aagctgctgt |
| | | | gtgagaccga |
| | | | gggccgggtc cgcgtggaga ccaccaagga ccgcagcatc ttcacggtcg |
| | | | agggggcaga |
| | | | gaaggaagat gagggcgtct acacggtcac agtgaagaac cctgtgggcg |
| | | | aggaccaggt |
| | | | caacctcaca gtcaaggtca tcgacgtgcc agacgcacct gcggccccca |
| | | | agatcagcaa |
| | | | cgtgggagag gactcctgca cagtacagtg ggagccgcct gcctacgatg |
| | | | gcgggcagcc |
| | | | catcctgggc tacatcctgg agcgcaagaa gaagaagagc taccggtgga |
| | | | tgcggctgaa |
| | | | cttcgacctg attcaggagc tgagtcatga agcgcggcgc atgatcgagg gcgtggtgta |
| | | | cgagatgcgc gtctacgcgg tcaacgccat cggcatgtcc aggcccagcc |
| | | | ctgcctccca |
| | | | gcccttcatg cctatcggtc cccccagcga acccacccac ctggcagtag aggacgtctc |
| | | | tgacaccacg gtctccctca agtggcggcc cccagagcgc gtgggagcag |
| | | | gaggcctgga |
| | | | tggctacagc gtggagtact gcccagaggg ctgctcagag tgggtggctg |
| | | | ccctgcaggg |
| | | | gctgacagag cacacatcga tactggtgaa ggacctgccc acgggggccc |
| | | | ggctgctttt |
| | | | ccgagtgcgg gcacacaata tggcagggcc tggagcccct gttaccacca |
| | | | cggagccggt |
| | | | gacagtgcag gagatcctgc aacggccacg gcttcagctg cccaggcacc |
| | | | tgcgccagac |
| | | | cattcagaag aaggtcgggg agcctgtgaa ccttctcatc cctttccagg gcaagccccg |
| | | | gcctcaggtg acctggacca aagaggggca gcccctggca ggcgaggagg |
| | | | tgagcatccg |
| | | | caacagcccc acagacacca tcctgttcat ccgggccgct cgccgcgtgc |
| | | | attcaggcac |
| | | | ttaccaggtg acggtgcgca ttgagaacat ggaggacaag gccacgctgg |
| | | | tgctgcaggt |
| | | | tgttgacaag ccaagtcctc cccaggatct ccgggtgact gacgcctggg gtcttaatgt |
| | | | ggctctggag tggaagccac cccaggatgt cggcaacacg gagctctggg |
| | | | ggtacacagt |
| | | | gcagaaagcc gacaagaaga ccatggagtg gttcaccgtc ttggagcatt |
| | | | accgccgcac |
| | | | ccactgcgtg gtgccagagc tcatcattgg caatggctac tacttccgcg tcttcagcca |
| | | | gaatatggtt ggctttagtg acagagcggc caccaccaag gagcccgtct ttatccccag |
| | | | accaggcatc acctatgagc cacccaacta taaggccctg gacttctccg |
| | | | aggccccaag |
| | | | cttcacccag cccctggtga accgctcggt catcgcgggc tacactgcta tgctctgctg |
| | | | tgctgtccgg ggtagcccca agcccaagat ttcctggttc aagaatggcc tggacctggg |
| | | | agaagacgcc cgcttccgca tgttcagcaa gcagggagtg ttgactctgg agattagaaa |
| | | | gccctgcccc tttgacgggg gcatctatgt ctgcagggcc accaacttac |
| | | | agggcgaggc |
| | | | acggtgtgag tgccgcctgg aggtgcgagt gcctcagtga ccaggctggc |
| | | | tcctggggat |
| | | | ggccaggtac aaccggatgc cagccccgtg ccaggagcct ggagggaagt |
| | | | tggggaaacc |
| | | | cctccctact gttggatgta tgtgtgacaa gtgtgtctcc tgtgctgcga tgggggatca |
| | | | gcagggcagt tgtcgggcag tcctgagtgg gtgttgcaca gactggtcca cagggctcct |
| | | | gaaggaagcc cctggatctt tggggtaaaa ggagggtggc ctcaagaaac |
| | | | aatgtctggg |
| | | | gacaggcctt tctggcctgc tatgtcttcc caatgtttat tgggcaataa aagataagtg |
| | | | cagtcacaga gaactca |
| 44 | human CRYAB | | aggaggagga ggcaccaaac ggcctgggtg gatagaaggg ggacaaggag |
| | mRNA (isoform | | gcacacccag |
| | 2) | | gccggcaaag agcagctcag tgagtactgg gtatgtgtca cattgccaaa tcccggatca |
| | 5′ UTR: 1-273 | | caagtctcca tgaactgctg gtgagctagg ataataaaac ccctgacatc accattccag |
| | (273-nt) | | aagcttcaca agactgcata tataaggggc tggctgtagc tgcagctgaa ggagctgacc |
| | uORF sequence: | | agccagctga cccctcacac tcacctagcc accatggaca tcgccatcca |
| | 93-149 (57-nt) | | ccacccctgg |
| | mORF | | atccgccgcc ccttctttcc tttccactcc cccagccgcc tctttgacca gttcttcgga |
| | sequence: 56- | | gagcacctgt tggagtctga tcttttcccg acgtctactt ccctgagtcc cttctacctt |
| | 3880 (3825-nt) | | cggccaccct ccttcctgcg ggcacccagc tggtttgaca ctggactctc agagatgcgc |
| | | | ctggagaagg acaggttctc tgtcaacctg gatgtgaagc acttctcccc agaggaactc |
| | | | aaagttaagg tgttgggaga tgtgattgag gtgcatggaa aacatgaaga gcgccaggat |
| | | | gaacatggtt tcatctccag ggagttccac aggaaatacc ggatcccagc tgatgtagac |
| | | | cctctcacca ttacttcatc cctgtcatct gatggggtcc tcactgtgaa tggaccaagg |
| | | | aaacaggtct ctggccctga gcgcaccatt cccatcaccc gtgaagagaa gcctgctgtc |
| | | | accgcagccc ccaagaaata gatgcccttt cttgaattgc attttttaaa acaagaaagt |
| | | | ttccccacca gtgaatgaaa gtcttgtgac tagtgctgaa gcttattaat gctaagggca |
| | | | ggcccaaatt atcaagctaa taaaatatca ttcagcaaca gataaaaaaa aaaaaaaaaa |
| | | | aaaa |
| 45 | human | Activate | CₘₒUₘₒUₘₒCₘₒCₘₒCₘₒCₘₒGₘₒGₘₒCₘₒUₘₒCₘₒ |
| | MYBPC3 | MYBPC3 | AₘₒGₘₒGₘₒCm |
| | type II motASO | mORF; | |
| | | Increase | |
| | | protein | |
| | | levels | |
| 46 | human CRYAB | Activate | GₘₒUₘₒGₘₒGₘₒAₘₒUₘₒGₘₒGₘₒCₘₒGₘₒAₘₒUₘₒ |
| | type II motASO | CRYAB | GₘₒUₘₒCₘₒCm |
| | | mORF; | |
| | | Increase | |
| | | protein | |
| | | levels | |
| 47 | human | | gcctgagccggggaag |
| | MYBPC3 | | |
| | mRNA | | |
| | Targct sequence | | |
| 48 | human CRYAB | | ggacatcgccatccac |
| | mRNA | | |
| | Targct sequence | | |
| 49 | human eIF4G2 | | cggagcucuauggagguggc |
| | mRNA | | |
| | Targct sequence | | |
| 50 | intact dsRNA | | GGGUUUAAUUACUGCACCGUAAUUGGAUCC |
| | sequence in FIG. | | |
| | 2, panel B and | | |
| | panel D | | |
| 51 | weakened | | GGGUUUAAUUACUGCCACCGCAAAUGCAUCC |
| | dsRNA | | |
| | sequence in FIG. | | |
| | 2, panel D | | |

“m” indicates a 2′-O-methyl modification,
“e” indicates a 2′-O-methoxyethyl modification,
“+” indicates LNA,
“s” indicates phosphorothioate,
and
“o” indicates a phosphodiester internucleoside linkagc,
“d” indicates DNA

Herein incorporated is the sequence listing file 1134-119 PCT.xml, created on Aug. 18, 2023, and size of 10,400 bytes.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. An antisense oligonucleotide comprising 8-50 nucleotides, wherein the antisense oligonucleotide is capable of binding to, and forming a double-stranded structure with, a target sequence in a mRNA of an anti-fibrosis gene, wherein the target sequence is located in a non-coding strand of a double-stranded stem structure downstream of, and adjacent to, a uORF start codon in the mRNA, and wherein the binding of the antisense oligonucleotide to the target sequence disrupts the double-stranded stem structure of the uORF and enhances translation of a mORF of the mRNA.

2. The antisense oligonucleotide of claim 1, wherein the antisense oligonucleotide comprises one or more modified nucleotides.

3. (canceled)

4. The antisense oligonucleotide of claim 1, wherein the anti-fibrosis gene is selected from the group consisting of GATA4, MEF2C, NKX2-5, TBX5, HNF4a, CRYAB, TCF21 and MYBPC3.

5. The antisense oligonucleotide of claim 4, wherein the cardiac fibrosis-related gene is GATA4.

6. The antisense oligonucleotide of claim 5, wherein the target sequence comprises the nucleotide sequence of SEQ ID NO:27.

7. The antisense oligonucleotide of claim 1, wherein the antisense oligonucleotide is RNA.

8. The antisense oligonucleotide of claim 1, comprising SEQ ID NO:8.

9. An antisense oligonucleotide comprising 8-50 nucleotides, wherein the antisense oligonucleotide is capable of binding to, and forming a double-stranded structure with, a target sequence in a mRNA of an anti-fibrosis gene, wherein the target sequence is located downstream of, and adjacent to, a mORF start codon in the mRNA, and wherein the binding of the antisense oligonucleotide to the target sequence enhances translation from the mORF start codon.

10. The antisense oligonucleotide of claim 9, wherein the antisense oligonucleotide comprises one or more modified nucleotides.

11. (canceled)

12. The antisense oligonucleotide of claim 9, wherein the anti-fibrosis gene is selected from the group consisting of GATA4, MEF2C, NKX2-5, TBX5, HNF4a, CRYAB, TCF21 and MYBPC3.

13. The antisense oligonucleotide of claim 12, wherein the target sequence comprises a sequence selected from the group consisting of SEQ ID NOS:28, 29, 30, 47 and 48.

14. (canceled)

15. (canceled)

16. (canceled)

17. (canceled)

18. The antisense oligonucleotide of claim 9, wherein the antisense oligonucleotide is RNA.

19. The antisense oligonucleotide of claim 12, comprising a sequence selected from the group consisting of SEQ ID NOS:9, 10, 15, 21, 45 and 46.

20. (canceled)

21. (canceled)

22. (canceled)

23. (canceled)

24. An antisense oligonucleotide comprising 8-50 nucleotides, wherein the antisense oligonucleotide is capable of binding to, and forming a double-stranded structure with, a target sequence in a mRNA of a pro-fibrosis gene, wherein the target sequence is located downstream of, and adjacent to, an uORF start codon in the mRNA, and wherein the binding of the antisense oligonucleotide to the target sequence reduces translation from the mORF start codon.

25. The antisense oligonucleotide of claim 24, wherein the antisense oligonucleotide comprises one or more modified nucleotides.

26. (canceled)

27. The antisense oligonucleotide of claim 24, wherein the pro-fibrosis gene is selected from the group consisting of eIF4G2, EPRS and MEOX1.

28. The antisense oligonucleotide of claim 27, wherein the target sequence comprises SEQ ID NO:31.

29. The antisense oligonucleotide of claim 24, wherein the antisense oligonucleotide is RNA.

30. The antisense oligonucleotide of claim 24, comprising SEQ ID NO:17 or SEQ ID NO:31.

31. An antisense oligonucleotide comprising 8-50 nucleotides, wherein the antisense oligonucleotide is a gapmer capable of binding to, and forming a double-stranded structure with, a target sequence in a mRNA of a pro-fibrosis gene, wherein the target sequence is located in a region that spans from 55 nucleotides upstream to 55 nucleotides downstream of an uORF start codon in the mRNA, and wherein the binding of the antisense oligonucleotide to the target sequence reduces translation from the mORF start codon and degrade the target mRNA.

32. The antisense oligonucleotide of claim 31, wherein the antisense oligonucleotide comprises one or more modified nucleotides.

33. (canceled)

34. The antisense oligonucleotide of claim 31, wherein the pro-fibrosis gene is selected from the group consisting of eIF4G2, EPRS and MEOX1.

35. The antisense oligonucleotide of claim 34, wherein the target sequence comprises SEQ ID NO:49.

36. The antisense oligonucleotide of claim 35, comprising SEQ ID NO:41.

37. A pharmaceutical composition for anti-fibrosis therapy, comprising: the antisense oligonucleotide of claim 1; and a pharmaceutically acceptable carrier.

38. A method for treating cardiac fibrosis, comprising administering in a subject in need thereof, an effective amount of the antisense oligonucleotide of claim 1.

39. A pharmaceutical composition for anti-fibrosis therapy, comprising: the antisense oligonucleotide of claim 9; and a pharmaceutically acceptable carrier.

40. A pharmaceutical composition for anti-fibrosis therapy, comprising: the antisense oligonucleotide of claim 24; and a pharmaceutically acceptable carrier.

41. A pharmaceutical composition for anti-fibrosis therapy, comprising: the antisense oligonucleotide of claim 31; and a pharmaceutically acceptable carrier.


SEQUENCE LISTING (51 sequences)

SEQ ID NO: 1 - RNA, 16 nt, synthetic construct
  source 1..16: mol_type = other RNA
  modified_base 1..16: mod_base = OTHER; note = 2-Prime-O-methyl nucleotides
  modified_base 1..16: mod_base = OTHER; note = Phosphodiester internucleoside linkage
ccaggattcaacctac

SEQ ID NO: 2 - RNA, 16 nt, synthetic construct
  source 1..16: mol_type = other RNA
  modified_base 1..12: mod_base = OTHER; note = 2-Prime-O-methyl nucleotides
  modified_base 1..13: mod_base = OTHER; note = Phosphodiester internucleoside linkage
  modified_base 14..16: mod_base = OTHER; note = Locked nucleic acid nucleoside
ccaggattcaacctac

SEQ ID NO: 3 - RNA, 16 nt, synthetic construct
  source 1..16: mol_type = other RNA
  modified_base 1..16: mod_base = OTHER; note = 2-Prime-O-methyl nucleotides
  modified_base 1..16: mod_base = OTHER; note = Phosphodiester internucleoside linkage
acgtattaaatccagc

SEQ ID NO: 4 - RNA, 16 nt, synthetic construct
  source 1..16: mol_type = other RNA
  modified_base 1..16: mod_base = OTHER; note = 2-Prime-O-methoxyethyl nucleotides
  modified_base 1..16: mod_base = OTHER; note = Phosphodiester internucleoside linkage
acgtattaaatccagc

SEQ ID NO: 5 - RNA, 16 nt, synthetic construct
  source 1..16: mol_type = other RNA
  modified_base 1..16: mod_base = OTHER; note = Locked nucleic acid nucleoside
acgtattaaatccagc

SEQ ID NO: 6 - RNA, 16 nt, synthetic construct
  source 1..16: mol_type = other RNA
  modified_base 1..16: mod_base = OTHER; note = 2-Prime-O-methyl nucleotides
  modified_base 1..4: mod_base = OTHER; note = Phosphorothioate internucleoside linkage
  modified_base 13..16: mod_base = OTHER; note = Phosphorothioate internucleoside linkage
  modified_base 4..13: mod_base = OTHER; note = Phosphodiester internucleoside linkage
gctgaattcaccccag

SEQ ID NO: 7 - RNA, 16 nt, synthetic construct
  source 1..16: mol_type = other RNA
  modified_base 1..12: mod_base = OTHER; note = 2-Prime-O-methyl nucleotides
  modified_base 1..13: mod_base = OTHER; note = Phosphodiester internucleoside linkage
  modified_base 13..16: mod_base = OTHER; note = Locked nucleic acid nucleoside
acgtattaaatccagc

SEQ ID NO: 8 - RNA, 16 nt, synthetic construct
  source 1..16: mol_type = other RNA
  modified_base 1..16: mod_base = OTHER; note = 2-Prime-O-methyl nucleotides
  modified_base 1..16: mod_base = OTHER; note = Phosphodiester internucleoside linkage
gctgaattcaccccag

SEQ ID NO: 9 - RNA, 16 nt, synthetic construct
  source 1..16: mol_type = other RNA
  modified_base 1..16: mod_base = OTHER; note = 2-Prime-O-methyl nucleotides
  modified_base 1..16: mod_base = OTHER; note = Phosphodiester internucleoside linkage
ggccaagctctgatac

SEQ ID NO: 10 - RNA, 16 nt, synthetic construct
  source 1..16: mol_type = other RNA
  modified_base 1..12: mod_base = OTHER; note = 2-Prime-O-methyl nucleotides
  modified_base 1..13: mod_base = OTHER; note = Phosphodiester internucleoside linkage
  modified_base 13..16: mod_base = OTHER; note = Locked nucleic acid nucleoside
  modified_base 14: mod_base = OTHER; note = Thymine
ggccaagctctgatac

SEQ ID NO: 11 - RNA, 16 nt, synthetic construct
  source 1..16: mol_type = other RNA
  modified_base 1..16: mod_base = OTHER; note = 2-Prime-O-methyl nucleotides
  modified_base 1..16: mod_base = OTHER; note = Phosphodiester internucleoside linkage
ccaggattcaacctac

SEQ ID NO: 12 - RNA, 16 nt, synthetic construct
  source 1..16: mol_type = other RNA
  modified_base 1..16: mod_base = OTHER; note = 2-Prime-O-methyl nucleotides
  modified_base 1..16: mod_base = OTHER; note = Phosphodiester internucleoside linkage
acgaattaaatccagc

SEQ ID NO: 13 - RNA, 16 nt, synthetic construct
  source 1..16: mol_type = other RNA
  modified_base 1..11: mod_base = OTHER; note = 2-Prime-O-methyl nucleotides
  modified_base 1..12: mod_base = OTHER; note = Phosphodiester internucleoside linkage
  modified_base 13..16: mod_base = OTHER; note = Locked nucleic acid nucleoside
  modified_base 14..15: mod_base = OTHER; note = Thymine
cagtcaactcctcttc

SEQ ID NO: 14 - RNA, 16 nt, synthetic construct
  source 1..16: mol_type = other RNA
  modified_base 1..11: mod_base = OTHER; note = 2-Prime-O-methyl nucleotides
  modified_base 1..12: mod_base = OTHER; note = Phosphodiester internucleoside linkage
  modified_base 13..16: mod_base = OTHER; note = Locked nucleic acid nucleoside
  modified_base 14: mod_base = OTHER; note = Thymine
ctggcagcttccctgc

SEQ ID NO: 15 - RNA, 16 nt, synthetic construct
  source 1..16: mol_type = other RNA
  modified_base 1..16: mod_base = OTHER; note = 2-Prime-O-methyl nucleotides
  modified_base 1..16: mod_base = OTHER; note = Phosphodiester internucleoside linkage
agcagggctggggaac

SEQ ID NO: 16 - RNA, 16 nt, synthetic construct
  source 1..16: mol_type = other RNA
  modified_base 1..12: mod_base = OTHER; note = 2-Prime-O-methyl nucleotides
  modified_base 13..16: mod_base = OTHER; note = Locked nucleic acid nucleoside
  modified_base 1..13: mod_base = OTHER; note = Phosphodiester internucleoside linkage
  modified_base 14: mod_base = OTHER; note = Thymine
tgcagtcgcctcctcc

SEQ ID NO: 17 - RNA, 16 nt, synthetic construct
  source 1..16: mol_type = other RNA
  modified_base 1..11: mod_base = OTHER; note = 2-Prime-O-methyl nucleotides
  modified_base 1..12: mod_base = OTHER; note = Phosphodiester internucleoside linkage
  modified_base 13..16: mod_base = OTHER; note = Locked nucleic acid nucleoside
  modified_base 14: mod_base = OTHER; note = Thymine
acccgctgccacctcc

SEQ ID NO: 18 - RNA, 16 nt, synthetic construct
  source 1..16: mol_type = other RNA
  modified_base 1..11: mod_base = OTHER; note = 2-Prime-O-methyl nucleotides
  modified_base 1..12: mod_base = OTHER; note = Phosphodiester internucleoside linkage
  modified_base 13..16: mod_base = OTHER; note = Locked nucleic acid nucleoside
  modified_base 14: mod_base = OTHER; note = Thymine
tgcaatcgcactctcc

SEQ ID NO: 19 - RNA, 16 nt, synthetic construct
  source 1..16: mol_type = other RNA
  modified_base 1..11: mod_base = OTHER; note = 2-Prime-O-methyl nucleotides
  modified_base 1..12: mod_base = OTHER; note = Phosphodiester internucleoside linkage
  modified_base 13..16: mod_base = OTHER; note = Locked nucleic acid nucleoside
  modified_base 14: mod_base = OTHER; note = Thymine
agtgcaatactcatgc

SEQ ID NO: 20 - RNA, 16 nt, synthetic construct
  source 1..16: mol_type = other RNA
  modified_base 1..11: mod_base = OTHER; note = 2-Prime-O-methyl nucleotides
  modified_base 1..12: mod_base = OTHER; note = Phosphodiester internucleoside linkage
  modified_base 13..16: mod_base = OTHER; note = Locked nucleic acid nucleoside
  modified_base 14: mod_base = OTHER; note = Thymine
gttcccaaattcctgc

SEQ ID NO: 21 - RNA, 16 nt, synthetic construct
  source 1..16: mol_type = other RNA
  modified_base 1..11: mod_base = OTHER; note = 2-Prime-O-methyl nucleotides
  modified_base 1..12: mod_base = OTHER; note = Phosphodiester internucleoside linkage
  modified_base 13..16: mod_base = OTHER; note = Locked nucleic acid nucleoside
aatcttttttctcccc

SEQ ID NO: 22 - RNA, 1892 nt, Homo sapiens
  source 1..1892: mol_type = other RNA
  mRNA 1..1892: note = mRNA
ggggacttggaggcggccggcgcaggggccgcgagaggcttcgtcgccgctgcagctccgggggctcccaggggagcgtgcgcggaacctccaggcccagcaggaccccggctgcggcgaggaggaaggagccagcctagcagcttctgcgcctgtggccgcgggtgtcctggaggcctctcggtgtgacgagtgggggacccgaaggctcgtgcgccacctccaggcctggacgctgccctccgtcttctgcccccaataggtgcgccggaccttcaggccctggggtgaattcagctgctcctacatcagcttccggaaccaccaaaaattcaaattgggattttccggagtaaacaagagcctagagccctttgctcaatgctggatttaatacgtatatatttttaagcgagttggttttttcccctttgatttttgatcttcgcgacagttcctcccacgcatattatcgttgttgccgtcgttttctctccccgcgtggctccttgacctgcgagggagagagaggacaccgaagccgggagctcgcagggaccatgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaa

SEQ ID NO: 23 - RNA, 1947 nt, Mus musculus
  source 1..1947: mol_type = other RNA
  mRNA 1..1947: note = mRNA
gcgccgggagcaggggacaagccggaggcccgcagagtggccgcccgaggctcagccgcagttgcagctccgcggactcacggagatcgcgccggttttctgggaaactggagctggccaggactgccgcttcgcttcgaagggaccgggccctctttgtcattcttcgctggagccgctctggagctagcagctgcgcctgggtgtgtagcaggcagaaagcaaggactaggcttctttagccggtgggtgatccgaaggcctgctcagggtgttcgagaccagcctggactgcgtctgggcacctccagcctctgggccctggaatagagtccgccctcccgcacgatttctggagcaaccgcaaatccaatttgggattttctttttcctgagcaaaccagagcctagaggtttctgctttgatgctggatttaattcgtatatattttgagcgagttgggcctctcctcgttttttgatctccggttgttttttttttggggggggggttagtttttgggtttttgttttgttttgttttgttttgatttttggtgacagttccgcacacccgcattctagttcttgtctgcctcgtgctcagagcttggggcgatgtaccaaagcctggccatggccgccaaccacggccccccgcccggcgcctacgaagcaggtggccctggcgccttcatgcacagcgcgggcgccgcgtcctcgcccgtctacgtgcccactccgcgggtgccgtcctctgtgctgggcctgtcctacctgcagggcggtggcagtgccgctgcagctggaaccacctcgggtggcagctccggggccggcccgtcgggtgcagggcctgggacccagcagggtagccctggctggagccaagctggagccgagggagccgcctacaccccgccgcccgtgtccccgcgcttctctttcccggggactactgggtccctggcggccgctgccgccgctgccgcagcccgggaagctgcagcctacggcagtggcggcggggcggcgggcgctggtctggctggccgagagcagtacgggcgtccgggcttcgccggctcctactccagcccctacccagcctacatggccgacgtgggagcatcctgggccgcagccgctgccgcctctgccggccccttcgacagcccagtcctgcacagcctgcctggacgggccaaccctggaagacaccccaatctcgtagatatgtttgatgacttctcagaaggcagagagtgtgtcaattgtggggccatgtccaccccactctggaggcgagatgggacgggacactacctgtgcaatgcctgtggcctctatcacaagatgaacggcatcaaccggcccctcattaagcctcagcgccgcctgtccgcttcccgccgggtaggcctctcctgtgccaactgccagactaccaccaccacgctgtggcgtcgtaatgccgagggtgagcctgtatgtaatgcctgcggcctctacatgaagctccatggggttcccaggcctcttgcaatgcggaaggaggggattcaaaccagaaaacggaagcccaagaacctgaataaatctaagacgccagcaggtcctgctggtgagaccctccctccctccagtggtgcctccagcggtaactccagcaatgccactagcagcagcagcagcagtgaagagatgcgccccatcaagacagagcccgggctgtcatctcactatgggcacagcagctccatgtcccagacattcagtactgtgtccggccacgggccctccatccatccagtgctgtctgctctgaagctgtccccacaaggctatgcatctcctgtcactcagacatcgcaggccagctccaagcaggactcttggaacagcctggtcctggctgacagtcatggggacataatcaccgcgtaa

SEQ ID NO: 24 - RNA, 1794 nt, Homo sapiens
  source 1..1794: mol_type = other RNA
  mRNA 1..1794: note = mRNA
gcagtcacagacacttgagcacacgcgtacacccagacatcttcgggctgctattggattgactttgaaggttctgtgtgggtcgccgtggctgcatgtttgaatcaggtggagaagcacttcaacgctggacgaagtaaagattattgttgttattttttttttctctctctctctctcttaagaaaggaaaatatcccaaggactaatctgatcgggtcttccttcatcaggaacgaatgcaggaatttgggaactgagctgtgcaagtgctgaagaaggagatttgtttggaggaaacaggaaagagaaagaaaaggaaggaaaaaatacataatttcagggacgagagagagaagaaaaacggggactatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatga

SEQ ID NO: 25 - RNA, 1098 nt, Homo sapiens
  source 1..1098: mol_type = other RNA
  mRNA 1..1098: note = mRNA
acatccagagctggccgacgggtgcgcgggcgggcggcggcaccatgcagggaagctgccaggggccgtgggcagcgccgctttctgccgcccacctggcgctgtgagactggcgctgccaccatgttccccagccctgctctcacgcccacgcccttctcagtcaaagacatcctaaacctggaacagcagcagcgcagcctggctgccgccggagagctctctgcccgcctggaggcgaccctggcgccctcctcctgcatgctggccgccttcaagccagaggcctacgctgggcccgaggcggctgcgccgggcctcccagagctgcgcgcagagctgggccgcgcgccttcaccggccaagtgtgcgtctgcctttcccgccgcccccgccttctatccacgtgcctacagcgaccccgacccagccaaggaccctagagccgaaaagaaagagctgtgcgcgctgcagaaggcggtggagctggagaagacagaggcggacaacgcggagcggccccgggcgcgacggcggaggaagccgcgcgtgctcttctcgcaggcgcaggtctatgagctggagcggcgcttcaagcagcagcggtacctgtcggcccccgaacgcgaccagctggccagcgtgctgaaactcacgtccacgcaggtcaagatctggttccagaaccggcgctacaagtgcaagcggcagcggcaggaccagactctggagctggtggggctgcccccgccgccgccgccgcctgcccgcaggatcgcggtgccagtgctggtgcgcgatggcaagccatgcctaggggactcggcgccctacgcgcctgcctacggcgtgggcctcaatccctacggttataacgcctaccccgcctatccgggttacggcggcgcggcctgcagccctggctacagctgcactgccgcttaccccgccgggccttccccagcgcagccggccactgccgccgccaacaacaacttcgtgaacttcggcgtcggggacttgaatgcggttcagagccccgggattccgcagagcaactcgggagtgtccacgctgcatggtatccgagcctggtag

SEQ ID NO: 26 - RNA, 3032 nt, Homo sapiens
  source 1..3032: mol_type = other RNA
  mRNA 1..3032: note = mRNA
gccagcagtgagtcggagctctatggaggtggcagcgggtaccgagtggcggctgcagcagcgactcctctgagctgagtttgaggccgtccccgactccttcctcccccttccctcccccttttttttgttttccgttcccctttcccctcccttccctatccccgacgaccggatcctgaggaggcagctgcggtggcagctgctgagttctcggtgaaggtatttcatttctcctgtcccctcccctccccaccccatctattaatattattcttttgaagattcttcgttgtcaagccgccaaagtggagagtgcgattgcagaagggggtgcttctcgtttcagtgcttcttcgggcggaggaggaagtaggggtgcacctcagcactatcccaagactgctggcaacagcgagttcctggggaaaaccccagggcaaaacgctcagaaatggattcctgcacgaagcactagacgagatgacaactccgcagcaaacaactccgcaaacgaaaaagaacgacatgatgcaatcttcaggaaagtaagaggcatactaaataagcttactcctgaaaagtttgacaagctatgccttgagctcctcaatgtgggtgtagagtctaaactcatccttaaaggggtcatactgctgattgtggacaaagccctagaagagccaaagtatagctcactgtatgctcagctatgtctgcgattggcagaagatgcaccaaactttgatggcccagcagcagagggtcaaccaggacagaagcaaagcaccacattcagacgcctcctaatttccaaattacaagatgaatttgaaaaccgaactagaaatgttgatgtctatgataagcgtgaaaatcccctcctccccgaggaggaggaacagagagccattgctaagatcaagatgttgggaaacatcaaattcattggagagcttggcaagcttgatcttattcacgaatctatccttcataagtgcatcaaaacacttttggaaaagaagaagagagtccaactcaaagatatgggagaggatttggagtgcctctgtcagataatgaggacagtgggacctagattagaccatgaacgagccaagtccttaatggatcagtactttgcccgaatgtgctccttgatgttaagtaaggaattgccagcaaggattcgtttcctgctgcaggataccgtagagttgcgagaacaccattgggttcctcgcaaggcttttcttgacaatggaccaaagacgatcaatcaaattcgtcaagatgcagtaaaagatctaggggtgtttattcctgctcctatggctcaagggatgagaagtgacttctttctggagggaccgttcatgccacccaggatgaaaatggatagggacccacttggaggacttgctgatatgtttggacaaatgccaggtagcggaattggtactggtccaggagttatccaggatagattttcacccaccatgggacgtcatcgttcaaatcaactcttcaatggccatgggggacacatcatgcctcccacacaatcgcagtttggagagatgggaggcaagtttatgaaaagccaggggctaagccagctctaccataaccagagtcagggactcttatcccagctgcaaggacagtcgaaggatatgccacctcggttttctaagaaaggacagcttaatgcagatgagattagcctgaggcctgctcagtcgttcctaatgaataaaaatcaagtgccaaagcttcagccccagataactatgattcctcctagtgcacaaccaccacgcactcaaacaccacctctgggacagacacctcagcttggtctcaaaactaatccaccgcttatccaggaaaagcctgccaagaccagcaaaaagccaccaccgtcaaaggaagaactccttaaactaactgaaactgttgtgactgaatatctaaatagtggaaatgcaaatgaggctgtcaatggtgtaagagaaatgagggctcctaaacactttcttcctgagatgttaagcaaagtaatcatcctgtcactagatagaagcgatgaagataaagaaaaagcaagttctttgatcagtttactcaaacaggaagggatagccacaagtgacaacttcatgcaggctttcctgaatgtattggaccagtgtcccaaactggaggttgacatccctttggtgaaatcctatttagcacagtttgcagctcgtgccatcatttcagagctggtgagcatttcagaactagctcaaccactagaaagtggcacccattttcctctcttcctactttgtcttcagcagttagctaaattacaagatcgagaatggttaacagaactttttcaacaaagcaaggtcaatatgcagaaaatgctcccagaaattgatcagaataaggaccgcatgttggagattttggaaggaaagggactgagtttcttattcccactcctcaaattggagaaggaactgttgaagcaaataaagttggatccatcccctcaaaccatatataaatggattaaagataacatctctcccaaacttcatgtagataaaggatttgtgaacatcttaatgactagcttcttacagtacatttctagtgaagtaaacccccccagcgatgaaacagattcatcctctgctccttccaaagaacagttagagcaggaaaaacaactactactatctttcaagccagtaatgcagaaatttcttcatgatcacgttgatctacaagtcagtgccctgtatgctctccaggtgcactgctataacagcaacttcccaaaaggcatgttacttcgcttttttgtgcacttctatgacatggaaattattgaagaagaagctttcttggcttggaaagaagatataacccaagagtttccgggaaaaggcaaggctttgttccaggtgaatcagtggctaacctggttagaaactgctgaagaagaagaatcagaggaagaagctgactaa

SEQ ID NO: 27 - RNA, 16 nt, Homo sapiens
  source 1..16: mol_type = other RNA
ctggggtgaattcagc

SEQ ID NO: 28 - RNA, 16 nt, Homo sapiens
  source 1..16: mol_type = other RNA
gtatcagagcttggcc

SEQ ID NO: 29 - RNA, 16 nt, Homo sapiens
  source 1..16: mol_type = other RNA
ggggagaaaaaagatt

SEQ ID NO: 30 - RNA, 16 nt, Homo sapiens
  source 1..16: mol_type = other RNA
gttccccagccctgct

SEQ ID NO: 31 - RNA, 16 nt, Homo sapiens
  source 1..16: mol_type = other RNA
  modified_base 16: mod_base = OTHER; note = Thymine
ggaggtggcagcgggt

SEQ ID NO: 32 - DNA, 30 bp, synthetic construct
  source 1..30: mol_type = other DNA
atgctggatttaatacgtatatatttttaa

SEQ ID NO: 33 - DNA, 30 bp, synthetic construct
  source 1..30: mol_type = other DNA
atgctggatttaattcgtatatatttttga

SEQ ID NO: 34 - DNA, 60 bp, Mesocricetus auratus
  source 1..60: mol_type = other DNA
atgctggatttaattcgcatatattttgagcgagttgggcctctccccatcgatttttga

SEQ ID NO: 35 - DNA, 60 bp, Rattus sp.
  source 1..60: mol_type = other DNA
atgctggatttaattcgtatatattttgagcgagttgggcctgtcctcattgttttttga

SEQ ID NO: 36 - DNA, 57 bp, Mus musculus
  source 1..57: mol_type = other DNA
atgctggatttaattcgtatatattttgagcgagttgggcctctcctcgttttttga

SEQ ID NO: 37 - DNA, 112 bp, Homo sapiens
  source 1..112: mol_type = other DNA
ggggtgaattcagctgctcctacatcagcttccggaaccaccaaaaattcaaattgggattttccggagtaaacaagagcctagagccctttgctcaatgctggatttaata

SEQ ID NO: 38 - DNA, 112 bp, Homo sapiens
  source 1..112: mol_type = other DNA
ggggtgaattcagctgctcctacatcagcttccggaaccaccaaaaattcaaattgggattttccggagtaaacaagagcctagagccctttgctcattgctggatttaata

SEQ ID NO: 39 - DNA, 112 bp, synthetic construct
  source 1..112: mol_type = other DNA
ggggtgacttaagctgctcctacatcagcttccggaaccaccaaaaattcaaattgggattttccggagtaaacaagagcctagagccctttgctcaatgctggatttaata

SEQ ID NO: 40 - DNA, 112 bp, synthetic construct
  source 1..112: mol_type = other DNA
ggggtgacttaagctgctcctacatcagcttccggaaccaccaaaaattcaaattgggattttccggagtaaacaagagcctagagccctttgctcaatgcttgagttaata

SEQ ID NO: 41 - DNA, 20 bp, synthetic construct
  source 1..20: mol_type = other DNA
  modified_base 1..5: mod_base = OTHER; note = 2-Prime-O-methoxyethyl nucleotides
  modified_base 16..20: mod_base = OTHER; note = 2-Prime-O-methoxyethyl nucleotides
  modified_base 1..20: mod_base = OTHER; note = Phosphorothioate internucleoside linkage
  misc_feature order(1..5,16..20): note = RNA
  misc_feature 6..15: note = DNA
gccacctccatagagctccg

SEQ ID NO: 42 - RNA, 20 nt, synthetic construct
  source 1..20: mol_type = other RNA
  modified_base order(7,11): mod_base = OTHER; note = Thymine
gccacctccatagagctccg

SEQ ID NO: 43 - RNA, 4217 nt, Homo sapiens
  source 1..4217: mol_type = other RNA
  mRNA 1..4217: note = mRNA
agtccctctttgggtgacctgtgcctgcttcgtgcctggtgtgacgtctctcaggatgcctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccctgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcagtgaccaggctggctcctggggatggccaggtacaaccggatgccagccccgtgccaggagcctggagggaagttggggaaacccctccctactgttggatgtatgtgtgacaagtgtgtctcctgtgctgcgatgggggatcagcagggcagttgtcgggcagtcctgagtgggtgttgcacagactggtccacagggctcctgaaggaagcccctggatctttggggtaaaaggagggtggcctcaagaaacaatgtctggggacaggcctttctggcctgctatgtcttcccaatgtttattgggcaataaaagataagtgcagtcacagagaactca

SEQ ID NO: 44 - RNA, 964 nt, Homo sapiens
  source 1..964: mol_type = other RNA
  mRNA 1..964: note = mRNA
aggaggaggaggcaccaaacggcctgggtggatagaagggggacaaggaggcacacccaggccggcaaagagcagctcagtgagtactgggtatgtgtcacattgccaaatcccggatcacaagtctccatgaactgctggtgagctaggataataaaacccctgacatcaccattccagaagcttcacaagactgcatatataaggggctggctgtagctgcagctgaaggagctgaccagccagctgacccctcacactcacctagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatagatgccctttcttgaattgcattttttaaaacaagaaagtttccccaccagtgaatgaaagtcttgtgactagtgctgaagcttattaatgctaagggcaggcccaaattatcaagctaataaaatatcattcagcaacagataaaaaaaaaaaaaaaaaaaaa

SEQ ID NO: 45 - RNA, 16 nt, synthetic construct
  source 1..16: mol_type = other RNA
  modified_base 1..16: mod_base = OTHER; note = 2-Prime-O-methyl nucleotides
  modified_base 1..16: mod_base = OTHER; note = Phosphodiester internucleoside linkage
cttccccggctcaggc

SEQ ID NO: 46 - RNA, 16 nt, synthetic construct
  source 1..16: mol_type = other RNA
  modified_base 1..16: mod_base = OTHER; note = 2-Prime-O-methyl nucleotides
  modified_base 1..16: mod_base = OTHER; note = Phosphodiester internucleoside linkage
gtggatggcgatgtcc

SEQ ID NO: 47 - RNA, 16 nt, Homo sapiens
  source 1..16: mol_type = other RNA
  mRNA 1..16: note = mRNA
  modified_base 4: mod_base = OTHER; note = Thymine
gcctgagccggggaag

SEQ ID NO: 48 - RNA, 16 nt, Homo sapiens
  source 1..16: mol_type = other RNA
  mRNA 1..16: note = mRNA
  modified_base order(6,12): mod_base = OTHER; note = Thymine
ggacatcgccatccac

SEQ ID NO: 49 - RNA, 20 nt, Homo sapiens
  source 1..20: mol_type = other RNA
  mRNA 1..20: note = mRNA
cggagctctatggaggtggc

SEQ ID NO: 50 - RNA, 30 nt, synthetic construct
  source 1..30: mol_type = other RNA
gggtttaattactgcaccgtaattggatcc

SEQ ID NO: 51 - RNA, 31 nt, synthetic construct
  source 1..31: mol_type = other RNA
gggtttaattactgccaccgcaaatgcatcc